SEQUENCE LISTING (5 sequences)

SEQ ID NO: 1 - DNA, 72789 bp, Homo sapiens
aaaagtccgtggaaagaaaaaaaccttgtcctggcttcagcttccaactacaaagacagacttggtccttttcaacggttttcacagatccagtgacccacgctctgaagacagaattagctaactttcaaaaacatctggaaaaatgaaggttagtataagcaacctcaagtattcttaaaaaattagggttggctggattgaacgtgatcagagttttctattgtttagaaggtgaagatttgtacataagtatttacttcctcaatgggttgggattggaaaattgttctaaatatactgtagaaacatttgtccttgcagagctggaagaattccagcaattatagccggatcagtggaaaacaatcaaaagctttgtggggcgagagaaaaaggatttaggtgtaaacaaaatcttgccatttctactatttagcaggaactgtgtgtatgcctaattctctttattttatgtagacttgggtaaaaatcgtatttggagttgccacctctgctgtgcttgccttattggtgatgtgcattgtcttacgcccttcaagaggtaagaactttctctgatttgctctatctgcctagaatagcttggaaaatgtactaatgcaagatcacaagtggttgaacatttcctaagtgcttatgttccaaagtaagtctgttatttctaagaagacatataagctcacagatatgttgttgtgcaagaccagaaatactgaggaatttaaggcatgggtaggaagagatttacagtgttatttcagcaatagaaaaacctaagaactatgctgcatcacccccacaacccccaaaaatatatgctaaatacaagctttgacaggcctttagaaacaacatttttaactaacattgcttttcattattaggtttgtctcgtggaaagtaaaaatgagtaagaatggaaaagttaaaacagaatctatggactttttttattactaaatagtctttccaaaaatttagggaggaagttgagaatacagatctcaggaatagctgtatagaaaaagcaaagttgttgttttagatgtagtagcactgcacatcaaattagaaaaccaaacattgttaagcaagcattttgatcagtcataaattggatccttttacaaaaaaggcttagattgacactgatctcactttgctaaaaattggaaggtactatgggctgatatcagagctagtttaaatataaagtaggactatgttgatagataatctagtgatattagggaaatagctttttatgaaaatatttatgaagagaaactcaagcttctgaaatggaaaaaaaatccagtaataacaggtcttatgtctttgtgtctaggaaaggggaaaaatttgtactaataaatactaaaagtttatgataacaaatgtgggaaatatcttggttaaaagttaactgatagtaaatgaagatcaataaaataacttcagctaaagaatgtatgagtcctgaatgactggactgttgctattaaaaagaacatagaaaaatgttttcgaaaattaaaaataatcatgagattttcacagaattacaaaatagaataggttaacccataagatgaagtcagctgatatcttcagtgttaatttcagtctttgtttctgctctctgccagcacaagattcttcttaggctgttgagaggtatgtagtgtgtgtacatatctttcataaattttcatgttaaaattgttttcagactcatctctgaactcagtggtcttggggccacatttccctcataccagcaattgtcaggtagtggattaggtcatatagatcctttgaatgttaattcattaaaaattagctcctgatttttaaagaaactcaaccttgaaattgatactctgcaccctcagaaataaatgttcttggaacaaaagtttgttaattttatagggcacttgcctccatgtccatagggaaggatgaatttcactatggaattcaagctataatttctaataagaaatgcattatatttacatatttacaaatgatgtatagagatacacatacacagatatttgtgtgatcacatataatctcaatccctaccctctttccaagtttagttatgtatagcttacatataaatatacgtcgtaaactatatatagtataaaagtcctatgtaatttataatacaattctattttcaactaacaggaaaattctaagaataaagtgatttcttgatagggtgaaataatcaaattaaactcaagtactcagtgaaatttgagtactcattcagaaatagaaaaatcataaataaggtgagaactgttgttttttggttcattatatcattctctttctcttctaaatttgctgaataacttctcagaatgtcagctaaacagcttgtgaatggcactagtcattaccctactttcctgagcaggacccggggaagtcagtgccacaggcagccctgaagttaggcgtggtggcctcctaattttcctcctttcttctcacatctcactggtcaatcaatcatctcccgtctggagtgccaggtccagtcctcactctcacatctgcccagtgcctatgagaataggatctgagatggcaaggcaggaaagggaaggaggaaggggtaaaaagaacagagaattcaaagctaagcgagctcctacttagcctctttctcacattcatcagaaatcccattacttcacatttgccttaggtgccctcatatcctcagcccagaattttatgcaccagtgtatgaactgtcttcttctgagaaacttaaagccacactcaggaaacattcattcattcatttacatattagtgtattagttctactaactccctgctcatcatcatgccagttgctgaggatatggaggtgcagcagtctagaattccctctgcaagttggggagtaagaatccagtgctttctgcacccaaagatgcatggtcagcaccacagataccagagaaggatttgtgttatttcacatcacgtctaaaggaaagaaaaaagagaaaggctcttccatcccaggagaaccagcttcaggattaaaagggtttaggaataattaaactggattataagaacaaatatgaaatgtagtagtgaaatgagaaaatccatttagctcacttacaaaatattaactataaggaaaaacttttttcgaaacttgtaaaatattccctttttagttttgccaaagtgtgaagcacacatgaatttctaccccttactcccaggttgtccttgccttcagcttacaccccttcaactgactcctttcctctctcctacctccattcactttttcttttgaggttcttcttcacaagcatcacccctcttcgtctgtgaatatgcaacaaggctgagagtggaaactaagctccttgacttccccttatttttgttcaccccaaagacaaacataaagaccttcttgcttcaagagatttaacagaggttacaaaagcaaaaaactagaagtgaaaagttacagaggttgcagaattttgcaggtaaattctacacaaacttgattcatgatttatattttctgaatacctggaccattcctccttggcttatagaaagaaatcataggaactatttgacctcgtttcagattcttttgagtatagagtgccccacatggtgatgaacaaatactcaacaaatgttagttattttttttctaaaatttctaacatgtagtaaatacttcataaatgatagcattttgtgtctgtgttcttccattgttgagaatatgtacttctacactcaacaatagcaactattccattctgtgtgatgtatttcaatttcaaaaatgtcttaattccgagagtagagagcctttatcccagaatatcatccctgttcctatccccaactttttttttttaaccaaaaacaacaaatgtttcagaaacttttgtgtctgaagcactcatacagaacactgatggctctagggcaaaagcaaaacaaaaggaaagaagaaagatataagaagctgaagggtggaggaatggagaataaggctgagtgagagtaaaaaaggaaagggtgggtggctgtgttggcttatggctgtaatcccagaacgtcaggaggccaagacaggaggatttcttgagcccaggagtttgagaccaggctgggcaacatggcgaaaccctttctctattaaaaaaaaaatacaaaaattagctgggcatggtggcatgcacctttcgtcccagctatttaggaggctgaatatcagaaaagtgtttgggaagttcaggaaaccaaaaggaaactgacagagctatgagaccaaaatcatttaaacaaaccaacaaagagaatatgtatatacatatgtaattgtgattctacatacatacacatatatatataatatgtagtgattatttagctgagtcttgccttttataatgcattgaagaaaatatgtgaaggccacagataatttgtatagcacagagatcaatattttttaagaagaaatatcatttggaaagtctgaattttagtcagaaatccgttgttgttgttattattagtattactaattagtattctattactactgtaacttcaacaatcacctttggtaaaataagatatacgtgagataaaatcatcttcaaaggagatataaatatgttagtagtaaagttaaaatattttcccaggactaacattcagtctatgaaacagagtccctcaaacatcttaagagttttaagcttattttttcactattcttatggagaaaatatgggaaagtctaaaaggttaaggaaaagtttcagaaagttcatgaatataaaagagctttttagtcaagataaagaaatcaagtttgcagtttatagttctctaatttgcacgtgaattttaaaatcacagatgaatcttggcaaactccctttgagtttcgccaaattctaaataatttaaaaagaaaaccaaatctttgtatgattctcacaatggagaggcgtttacctgtctcagatttccaaagcaaattgagttacaacttcaacgagaacatccagtcatagatataccttgaaaaccagattcaaaagtctgagccagaggtgtgcaatcaggactaaaagtttttaagaaccacagacagtcaaactgagccataaaatatagcaataattgacaaatgggatctaattaaactaaagagcttctgcacagcaaaagaaactagcattagagtgaacaggcaacctacagaatgagagaaaatttttccaatctctccacctgacaaagggctaatatccagaatctacaaagaacttaaacaaatttacaagaaaaaaaaaacaatcccatcaaaaagtgggcaaacgacatgaacagacatttctcaaaaaaaggcatttatgcaaccaacaaacttatgaaaaaaagctcatcatcactggtaattagagaaatgcaaatcaaaaccaaaatgagataccatctcatgccagttagaatggtgatcattaaaaagttaggaaacaacagatgctggagaggatgtggagaaataggaatgcttttacactgttggtgggagtgtaaattagttcaaccattgtggaatccttgtgattcctcaaggatctagaaccagaaataccatttgacccagccatcccattactgggtatatccccaaaggattataaatcattctgctaaaaagacacatgcacatgtatgtttattgcagcactgttcacaatagcaaaaacttggaaccaacccaaatgcccatcaatgatagactggataaagaaaatgtggcacatatacaccatggaatactatgcagccatgggacatgcagggacgtggctgaagctggaaaccatcattctcagcaaactaacacagaaacagaaaaccgaacactgcatgttctcacttataagtgggagttgaacaatgagaacacatggataaagagaggggaacatcacacacagggcctgttggggggtcgggggactaggggagggatagcattagaagaaatacctaatgtagatgacgggttgatgggtgcagcaaaccaccatggcacatataatatctatgtaacaaacctgcacgtcctgcaaatgtatcctagaacttaaagtataatttaaaaaattgcaataattaatacagtagcatgtaataaaacgatctcataagacagtcaaatactaaccagtctctttttatttctcattgtatagagaaccaagaacttatttttcatttccagtatcctttttaattgactaatatgcagtaagaacagaataacttacaatacttattaatatttattcccattaataataaaaacagtattatttagtatcaaaattaaaattttaaatacaggttatgatttatacttggtggaaaaacagatgttaggaccttttaaggaacaggatataacaattgtatgttaaaaaagaaaacagataacaggccaggcgtggtggtaattgcagcactttgggaggctgagatgatggatcacttgaggccaggagttcaagaccagcctagccaacatggagaaactccctccctacaaaaacacaaaaattagcagtggtggctgacacctgtaacctctgctactcaggaggctgaggcagaattgcctgaatctgggagacagaggttgcagtgagccgagattgtgccattgcactccagcctgggcaacagagcaagactctgtctccctgcccccccaccaaaaacaaaacaaaatcagataacaaatgaaataggcacattgaaataggcacacagaaagatactaactaccaatacttaagaagaaacagaaaacatgagtggtgctataacaccaaaaaagattgaattagtcattttaaaacttcccacaaaaaaaaaaaaacaaaaacataaaaacttcctaagctcagatggcttcactgacaaattctaccaaacacttaaagaagaattatcacaaattcttcacaaacgttttcaaaaaatgaaagaaaacacttcccaactcattctataaggtattacccgcattactataatgccaaaaccaaagatgtcacaagaagagaaaattataaatcggtatctcttatgaatatggatgcaaaaatttcccacaaaataccagcatgccaaatccagcaatatataaaaaggattacacaccatgacaaattgggatttagcccaagaatacaaggttggtttatcatttgaaaatcaattaataaactttattaatagaaaaaaggacaaaaacctcatagtcataatagacacagaaaaaggatttgacaaaatgcagcaccctttcatgataaaaacacttaacggattaggagtagaaggaattccccaaccttatcaaagacacctacaaaaaaaaaccatagttaacatcacacttaatgaaagctaatgtcacactgtgtccggaattggtgggttcttggtcttgctggcttcaggagtgaagctgcagaccttcacagtgagtgttacagctcataaaggcagtatggacccaatgagtgagcactagcaagatttattgcaaagagcgaaagaacaagacttccacagtgtggaaggagaccccagcagagtgccactgctggctcaggcagcctgctttattctcttatctggccccacccacatcctgctgattggtccattttacagagaactgattggtctgttttacagagagctgattggtctgttttgatagggtgctgattggtgcgtttacaatccctgagccagacacaaaagttctccacctattgcctagatacagagtgtcaatgggtgtatttacaaaccctgagctagacacagggtgctgattggtgtgttcacaaaccttgagctagacacagagtgctgattggtgcactcacaatcccttagctagacacaaaggttctccaagtccccactaacctcaggagcccagctggcctcacccagtggatctcgcaccggagccgcaggtggagctgcccgccagtcccgcgccatgcgctggcactcctcagcccttgggccatccatgggaccgggcactgtggagcagggggaagtgcttgttggggaggctcaggccgtgcaggagcccatggcggagggggaggggcaggaggtgacagggggcgggcggtgcggagggggtgacgggggactgggggagtggggggagtgggagagactcaggcatggttggctgcaggtcccgagccctgccccgcagggaggcagctaaagcctggcgagaaactgagggcagcctcggtgggccagcactgctaggggacccggttcgccctccgcagctgctggcctgggtgctaagtccctcactgcccgggccgctgggagtgtggggcccaccaagcccacgcccacccagaactctagctggccggcaagcgtggggtgcagcccgggttcccctccctgcctctccctccacacctccccgcaagccaagggagccggctccagcctcggccagcccagagaaaggctcctacggtgcagcggcgggctgaagggctcctgaagtgcggccagaatgggtgccgaggccgaggaggcacccagagcgagtgagggctgagagggctgccagcatgctgtcacctctcaacactgttttcctgctaagatcaataagaggacttgatgtcctctctcatcgcttctgttcaacattttatcagtggttctagccaaggcaattaggcaagaaaaaaatatataaagtatccagatttgaaaggaagaagtaaaactctttctactgacagataacacaatcttgtttgtagaaatcctaaggaattcactaaaaaactattagaactaacaatacagttcagtagggttgcaggatacgagatccatgtataaagatcaatagtatttctatacactctcaatgagcaatccaaaaagatctaaataagtggaaagacatcccatgtttatggatcataaaacaatatttttaagatggcaatactgcctaaatttatcaacagattaaatgtaatctctatccaaatcctggatagctttctttttttgcagaaagtgactagctgatcctaaaattcatacaaaaatgcaagggacccaaaatagctaaaacaatattgaaaaagaataataaacttagaggaatcacacttccttattttaaaacttactacaaagctccgtagtcaagacagtgtgttactggcataaggatagatatatagatcaatggaatagaattgagtgtccagaaataaagtcttttacttagttaattgatttttgatgaggatgccaagacaattcaacgggaaaagtcttttcaacaaatgatggtgaaacaattggatatctatgtgcaaaaaaagatgttggacccaattcctcacaccacacacaaaaattaactcaaaatggatcaaagacttaaatgtaagagctaaaaactataaaactcttaaaacatacagatacatttttatgtttttgagttaggcaaattttctaagatatgaccaaaaatgcaagaaacaaaagaaaacatacataaattggacttcatcaaaatttaacattttagggtggccaagaataccatttaaagagtgaaaagactacctagaaaatgagaaaaaatatttgcaaatatatattcgataaaggccatgcatctagaatatacaaagaattctggcagctcaataataaaaagataaataactccattaaaaaatggacaaaggacagacattttgctaaggaagatatacaaatgactaataagcacatgaatgatgctcaatgctatcagtcactgcaaaatgcaaaacaaaaccacaacaaaataccaggaatgaaattgcagaggagcaattaggcatccgttaaagcctaaagattgtgaaatggcttgtccctgggggtgcagtagaaatagagacaagcagtagacagggttggcaggcctgggtgttggttaaggaagaggaaggtcaaggatcacaccaaaatgtttgtattgagaaactacatgaatggtaggtagtgccatttacttggagaaatttggaagaggagcacagaattttttttcttagcattaaacatattgattttgaagtgcttgtgagatatgtaagtggggattttaaagaggaagtttaatgtacgtgtctggagttcaaaagacaatccagtgaaagtcaggtgtggtagtggggtgagggggtgggtcagcccagggatatgtagtagaaaccttgggagaacgaaatgacctaggaaagttgctcttcctccagccccatgtagaagcagtaacagccatgccaaaatgtcaagtagagattagaggaaaatgagctggcaaaggagactgatgtcttcaaaaataaacaaggcttgctttcatcttcagagtcaccagagggtcatttgccattcatgttcttacacattaggttgattatatcttatgatctttgaaagccttagttatccagaaaaataaatgaaccctaattaaaataatataacataacaataaaataacacacaaatgggctatttttaaaagcccggcaaatcctttgaataactcatgttattcagtattgttcatagagaaaaccatggaaaattgctgttagagctgagacattcttcaagtaggcagtaataatagtagcagtaagtagttttataatagctagcaacctatttgctttgaaaaagctcaaattgcctcactttctcttaacctaggttttgaggtgtaattattctacccctttgtgtgttgcttggttcttgcctatttttgtgtttgttgtatgttgttgttttattatgtgtacacttatactttaattatagtatagtggcaaagtatctaaaaccagatgcagctgaatttaaaccccagcttttctatgtattaggcaagttagctcaccttgctaattttcatttcctaatctgtaaaatggtgtccacaaaagtatttacctcagatggctgctgtgaggatttaacaggactatacatgtagagtacttggtatgtacaagaacttataaattataacactttctattactcatctaaagccacctcatgtactttttttttcagagttgtcagtcttgttccaatttgatcatattagcttgtaaatagtatatttcccatcctaatacttcctgtccttgcccctgtggtgctcactccggaaacaaacaaaacccggtgtgataattggagatgatacctccccacaggtagcacctctgcagagccagcaggaagatggatgtgttttccctgagagccctagaccctttccttggtatgacctcatatgcatcaattacaaatcaagtgcaaaagtgagaacaaaaggacctgccagccatttgtatgccacttaattttctcatttggtttatgctgaaggttagaactaacatccaatgtctctgctctaacacccattcctaatttttcagtactagacaatcaaatggcagaaacagacacagaagcctaattgaagcacaacgttttaagaaagaattaagagaatagtaaatcattacttgcttgtgaatcatttctgtgaattgtaatttcttggattcataattgaaataattttcagctttgctttaataagctgaaaatgcaaataattaaagcatagttaatgagagtgtttaagcaatgtatgagtaactatactaaagatgggccaaaacgaagttaagggcactggcaaaaattaagagctaatcaagtaagtgttaacatatacagaggagagatatgctttcagaattcatgtttacttctgttcaaccattagaatttaaaaattatagatgtcaaagctaatcttccaaaatcatcaagtaattacatttaatattaaaacattatttggaaggggacatgaaaattcatttgttgatccaataattattaatggaatattttctgggtgactagaacatttagtgtatttgatgctgtggagatatattttatactgatttacacacagacacacacatacacacattccaaacactagaaaataatgaccccagtgctttttccataatcttacttaagtctagtctggggtagaatcagtccccttgccttttgtccacttttccaggtctgggaaagggaatatacttaaagaagagtgcatttgaaggcaatgagcaaggtcattgaacagggggacaaacattaaaggaggccatttacattccttggaagccatggtcaacactcagttatgtggaatctcttctccatgacatatggattgctctgcaggccagcctcacccacctaccgtgagtttgtagtcaaggaatgattcctataggcacaggagtgcattgagcaaaatagattcagtgaaatcattaaattgagattctgaacctttgcagaagaatcatcaaggagactgggtaaaaactaaaactaccaggaccccaccccccagagatttggattcagtaagtttgagataagtactaggaatctgttttttatataagcacactccaaggggctccaaggaccaaatttttataaatatttaaatgaaggcaaaagagtctacatatgaaatttacaacatgtaatagagggtagaaagtaagggaagatatattctcgggggaattattcaaaacaagcgaacacaaaatctgtcaagttggttttttttttaattcagcaaatatttattgagctcccatgctatgtcaaggactgtcctagcagctgaacgtataatagtgagaaaaatcaatcactgcttctacttttatgggtggagacctatgtcttatacaattctttgccacataaatatagagatgtgaaaaaattcatgttcaataaatttagataaatgttgagaaaacaaaaacccatatgtacaaagaatcaaagcttggattgaaaattctaagtaatctttatgttttattcttattctaacatgtgacttaatgcaccatctcaaaggagacacattacttcactgtgtttatgaacacgtggtcatgtttagcaggaagagaagaatgcttaatttgaatttctctgagtatattggggatgaacttaatgtacccagcctgacatcaacataattacactttctaagccctggatacatgacatatgatacggtttcaaagctaacgtgaggttaggagtaatcatcccagcccaatctgtgcctgaaatccattcccacatacatgtagctggcttatctggatatgttattcatacatccattcatcacttcttgggtggtggtgaggattattttcattatggcaattagtagtacaattagtacatgcttaaaattgctttacagattttttgagtcatatgctacgttctctatatcgattgttggcatttatagctactatgcactcatataaaattaattgctaactaggaagtaatatgaaatatagtatcaagtccaaccttcctggataaactaaattagaggtttaaaggaataaataattaattcttagaatctttttgagaactctgataagatcaaaatgctttttaatgatacgaaaaggaaaatgagtaggtatttaaaaagtaatttgaaactgttgttttttatgttttatttattcattgtacaatattagtacaaattgcacttatgattttaattgtaacagttcagttatggagctaggcaatagacaaaatgttcaagtataactgatgaacttcattaaactagaataaaagtaaagctcaattgtatatactaatagccttgtctggaactgtgtttctcaaactgtggttttagtagatattcagaagcaaaaattcccagtggacaaatctgcttgggaaacattgatctaaaacatttatttgaaaaatactgatgagttacgataatgctaaaaggtaacatgtattgactgctaccatgtgccacacatagagctagatgttttagttgaattatctcacttgatttagacttctatgaaaatgaagattaattatagctataaatgtcttaattaagcataacacaagtaaggaaaattatatgcaaagatgcttaaataggaaaaactatgtcaccagccataactgaagcattatttatactttttaaaaaataactaaggttatattcatttattgaattaatatcaaaaacattctttaaaaaaattgttatcttggtatttacagtaaatggactttatacatttttaaattttccatctagatattcaggattgtgtaggcacaaactaaatgtgtctcaagtggaaaataagttccactatgaatagtgttataattttccctttattacattagaatcagattgcaacttgctcttaggaaagttaaataaagttgaattaaatacaatacttggcaattattttaatgctattgtagtaaatggtcttttattcactgaatgagaattacaaaatcaatggtatgaattctacataataccatttcattaaacagaagatatttcattacaaataactgtatagtgtacacaaccttgtttaggatgcacagataacacagaaagataattatttaaaaaatagaataacaattctattaagtattcagtgtctagttgcaaaagggctcaccagccactattcttacagccatggaataagttgctgcaaattaaggaaagaaataaactcatggtcctaatcaaaagtgtggagcatacatgcatcaatgtaaatcagttaaaacaacagaagatgttattattccactttattattttgctcttactacatacatagcagaacttaatgtaactataagattaatagagaaggttgagtttcagatttcaaaatctagcatggtatagctggaagagattgccaacaccttctagtccagtacctcataatatgagtgagcaagcctaggcaccagagatctacttaccaaaaaccacacagggaattcagaaaaggcacacatctcatccagccagccacccaaccagccatccagccatccagccagccttcatttcatgcattcatttgttcaacaactatttattagatgcctgtatgtgctagacacttgggatgtgctatgagaaaaacaagacatgattcctggccttatggtaactgttttgattatacagagggaggtgggaaaacagtaatcaaataatcactcaaatatacacacgattacaaaccaagaggagtgttgtaaaagcaaggaacatgctctacggaaatgtataaaaggaaacctgatctaaaccagggggtcagaaagggcttctcagaggaagtatatagcttttggattgagcattaaaggagagagactagaaaaggctgtagacaccctacagaaagagtatttgaggaaaagaaagactggcttgttagtggctagaaagtaatgagagaggagcaagaagagctggcagggtagactgccccacaccagccggtgtctgtagtccaaccagatcatcatctgtggtctttactggaagagtcatggagggctacgagttttatagcaggataaagttaaacagctcccatctttgacagttttgttctcacctgggcaaataccagattcataaatggcattcaaggtataggatcttcctataagttttagtggcctgtggaaggggtaaggtgggagagatggagaatggcaatattgataaaaaagggcattgggctttggttgccttattatctgtctggtgtatctgactgcttcagggctctgtaatgaatgtttgttctgagaataagatcatgtttgaaaaacacaatggttttgaagtattcaaatatagattgaatttgtttgtggtttgtgagctgcattagattagtttttctacagaaaaccctctactcttggaatttctaagatattttgcatagatcaaggggttttcttttcttttggtctactttctttatataaaaatccatagtatgagaaaattacctacattcataatgtctttcttgagatttctgaaggtaagggttctggccaatctcatttgaccaactgggatgctaaaatacatcattgataaatcagttgaaatctgaaccaaacctatatgaggaaatacagtgagctgcataagagactgggattttagccaaacctacatggtctgtaatcccagctctgcaaggactagagttgttggaacctcatctcatcttccttctggaaaaccttgacaactcttcactgcctgaagaatcatgccccagctactctgcatggccttcctctgtaatctttatcttcctctacagtttccagccagttttcccttccaggcagtgaaactacctccctaattcttaattactgtatctacaaaaaaggagatactactattaataataatagttatgatgataggagttcaacaaccacttagcaaacagcagatcttattactatataagaaagaacagatgaaggccaggcttagaatttatttgagttgttaaccttctttttaataaggatgtatttgctcttcccccaaagttcataactctgaagaaaatacaatgagagcactcacactgaaggatattttaaatggaacattttcttataaaacattttttccaaactggatttcaggtaagtgcattatttatctttaggggttttttttatgtattttgtttggtttttattttagtgccaatacatctctatagtaactgaagccgtatcagttgaaatgtacaaaaagaacaattatctgaatgattcaagtgctcaaaaaagcgcttttgacaaaaccattcatactggatttgcgccatagtaatgaggccaatagagttttaaatagagaaggaaaagttgaaaatgcattgttgaaataatttgaacttctgtaatcacttttgagaatggtagatatattcctatttaggagaagtcacaaaaattttaccaaaagagaaattccattattaatctggataatctggaaaacagacaaatctcaatctgacaatgaaccctttaagggtcatgaaatcggtattttcatacagcttttcaaaataggatgttataaagcaaatctaatcaaataaatagtgagatcaactaaatagtctatgacattttagttgagtatcttaagtgtcactgaacaaaatggaatattttaattttttctctttagaaaataaaattaatacaaagtgagttttaagttgaaccaaacacaacttaaatgtaatttatgtttattactgcaaaagaaatatacacttcaaactagtttatcccctttgtaacccagagaaaacaaaattgtgttcctaagcaccctgaatcggctaaaatagggctgctgtttgcaaatgtggcatctttgtgtgaagtagaacaaagcattctaccttcttcctctaccaccaacttactattttacatatcacaaagcaatatgtactggacagagaggaaaaacaaagtaggtctgtgaaatagagaggtttcttttgttaggaatgtatttcatgttaagagagatcatttacattttgttttctttcttgtttaaaggacaagaatatcttcatcaatctgcagataacaatatagtactttataatattgaaacaggacaatcatataccattttgagtaatagaaccatggtatgtatccaacatcatcttccccctcagaaacctttgcccactcttcattgaccaagaaacagagttcaaagtcttagcacctgatctggacacaacttgccttcccagctccatactccactagagtcttcccgttctgtctgctagtcccattagactactgtctttgccttcttgtaaatcaactagcatctttcctcctctttgaatatagtatagcataagataggagttaagaactcagtccttagtgtcagccaattgaggttcaaatcctggctcagtcccttactggtcgtgaaaggttaggtaaggggtttcactacagcaatcttactatgcctcggtattttccttggtaaagtaggaataataatgcctcttttggttaataaaagaattaaatgaaataacatttgcaagtgcttagggcagtgcctaacatagaataagtgcttaataagtggtagctataaagtatgggcacttaactgttctgtcccactgggctcccagtcctccaactttctccaccctgaaaaacctactattcttttgaaaccaagtttagataatactctctttctctttgcaatcaataatcctctcatttgaattcaatcatttctttcttaataattttgcaattgctatattgaatctcttttatagaacatcctaatttgtagtatgttactccttagaaagtttatgtttcttgaaatcacaaagtatatattatctcttgcatccttctcccatagtatctagtaaatttctttgaattatagttaagttccacaaaatatttttttcaattgcattgaaagtctatgcatgtaggagtatgttgataatataatattcttttaaaactacaaatttacatggttctttgttatcttttctgtattctaatcaacctcttcataatttcagaaaagtgtgaatgcttcaaattacggcttatcacctgatcggcaatttgtatatctagaaagtgattattcaaaggtatcaactatttaatttggttatatccaattgtatctcctactaatccacaaggtggtttaaaaaaagtgagagaaagcaagagtctgtctttatctctaaatattcaggccctcgtcaccaataatcttcagcatgatatgtttcaatcttatggagcttgtttgttttaagcgtgaaagtgctttaaggaacagagtccaaaataatacttgcatcatttattaacaacagcacaaacatacttaacaagaaaacaaagagctttaagaagaattataagctaaatattcttatttatttttcaattattatttttaaaactgtactcaaagaaaggcaagcattttgcatcaaaaatacatgttgaatgaaattctctttacccaattttaaagggtataattagaatgaattatccagatgcagggtaatccttataggctgtggaaatcattgtggttttacttcaatacaggtaaaacatgaaaggaaaatttctggggtagagtatgcaaagataagctttttatgttaggaaatttctttggatatgctttttatggagtgagggttgaatcactgctgagattagttctggaacttgcacaaacggcacaaaagtccacagtaagactctttaaatgaagactcatttggaattgaaacatattcttcctagtgccttgtgaaagtagtccttttataattctgaactttgaattggatgctatatgattatacattattgatataagctttttaacaactgcaatttgttttttgtctttcttctttctgacagctttggagatactcttacacagcaacatattacatctatgaccttagcaatgggtaaatcacctctgtttattaatactacttaaatctaatacctagaatgttttcattccaaactatgatttggggttaagaagactttatcttcttaatgatctttgtttcttactttttagtaatacagtatgtatgtacccataaagcttacaaaattcaagaaaatacttatatgggatacccacctctttatttgactaatattttatatttttattcacaatatagttttggattttctgtttttgaagatcagattaaacatgtgctatttatggaaaaaacatatataggaaagcacaaaaacattcttggaaatgagacactatttttaggagagtggctatttcactgatgggaggaggaagggcgggagagagatgtagcattagttgtgtctgaacacgtactccattaactgttttaaaattaattattataaaaatgacaaaatgttaacattatttttaaaatagaaaaatatctgacgtgtaaaatactaaatgttcttttgaatttagttggtagataaattgtcagacaagtaccattttccatatatatatatacatataaactgtttgaaacatttaccatttaaaagaaaaaattaaatgaatgtgcagataggcgaagcattgacgaagagaatttctggcagcaagaagaatatgtaaaggcctaaaatggggagggagagcattgtgcgtttgagaaatgcaaagtaggtcaatttgtttgaaattgagggaggggcctgctgactagtaggaggtggtgtaaggttggggaacagccaaggccaggaagcataggccaaataataatgaggaaataattaaaatgagtttagatctttctttctgatgacaataaggattcattcaaaggttttaaatggaaaatgacatgatcagattagcactttagaaaaatcaccctggctaagtgtggacaatgaattgccttagctactcatcttaatatgatatcttgtttgctgttatataaaaggggaaataattatatggcttgaagaggtattgaaaaaaaatcttctggtctatttccccttttgtctagcaagagcacaaaaattttttaaaaaattatctatctacttctttcaaattttcagaaaagaattactctgataaactgtcaaattaagaaacaggaactgctttttaaagtagaatctaaaatccatctaatgcattctgagtccttcttgtctccgaatccatatcatcaccaaatttaagaaatatttattgagccccttctatgtgaaacacatataacaatgagatgcaaatgagcttggggcaccagctctactcttaagaaacgttcgattagtgagtggggaatcttagtctttcagtccagtttctcaaagctctcatcatgtctttgtttttcttttaatcattttgaaattcttttgtgaggctcgccaaattgaacagcattcaaaaagatgataatcctaatattttaatcatgggttatgtaaactttatggcctaaaatcacattaaacatttagattctctgcagtgaatttaatcaaatcactggttttgtttgtttctttgttttctgcacaactatctagtatttcagattttttggtgggggggtggtcaggtaaatacagcataatttaggaagtgaaggatggatggcaaacttgtttgagattctaggcatcgctgaataccaccaatttcaagtcttttatatgcctttgaagactctataaaaatctagcatagagaaaacaaaactactggtttcaagctaggtaggcaaaactgaggagatgaacgcctttgtaaagcatcctccctcgccttttctcatacctttagtgactcagaaaacagaaaatggcagctgtgatggcttgagacatgcgctgagatttttagcttagtgctgcttgtgtgattgcatagataattccactgtatgtagaaaactgagacaatgttacagtatgaaaggtaacatgagcactagcttgagaacaaattgtgaaccaggtgaagaatgtcttcttatttttttttttgagacagggtctcgttctgtcgcccaagctggagtgcagtggtgtgatctcggctcactgcaacctcctcctcccgggtttaagcaattctcccgcctcagcctcctaagtagctaggattacaggcttgtgccaccactgctaatttttgtgtttctgtggggacggggtttctccatattggccaggctggtctcgaactcctggcctcaagaaatccgccgcccaaagtgctgggattactggcgtgagcgagccggccaggcctgggtcaatcttttaactgggctcttgcatccctttcaacatgttcccgatcacccaggggcctggtgctcatgtactgccctctggcggccaggactctcattgcagcaccaatggtcactttccatgggaatcactctccccactcaaaactatgccatgttcccttgctacttgctaggtctcatgcaggcacctaaaaagttacatcaaggcagtgtgtgttacagatcttcacaattgcccaaatattaaaagaatgggaaaggcagaaatgtcaggcacatgttagctccaggttggagagctttttttggaggggttgggcaggagtcttttttgaggctcttcaaattgtgcttgggcggcaggtcttgctctgctttcccacgaaggcagtctcgatgaaactgttaatacaatcttcaagatgacaaaagtcaaaaagtaagcatgacatcctacgactctgcaacacttataaaagtctgaatgcattcttgctgaagaactacaaaagctccacaatgcaagtcacttgagtgcttgcagagtgactctaagcatcaaagtgagagttttggccaatagactatttgaaatttccccatgggtggaggactttacaagcttaaggcatactgcccagtatacaatccgtttttcagtttagcttgagatcagatgttcctttccttttctgatttcaagtcagatccctggggagattgtcaaatggcatcttattttcaaaaatttgaaatgcatatgcattcacagagatgggggttgtgaatgagaatgtagaagagcacatactaaacttcagcacaattatagaatttcagagctggaaggcacttcagtgaccatttcagtctagtgtgtgcaaattttacaatcccaggggttaaaaggacttgagtttaattcaaggtctgccaagaactagcaatgtggtcttaaaaagttctatcagctctctaaacctcatatttactttctataaaatgagaggactgtattagataatttctagagatcttctccactttgacagtctgtaagtatttaatccattcacctcactataaaggtcagaaaactgacacccagtaaagttaagtgacttttaagtaactactggaagaacagcaatcaacacctatgtctccagttcttcattcctgcattcttttcattaggtttgtgggtgcagagggaatgatccttaattttcattgtttgtttattttttctgttcatacagattattatttttttgcattaaacagcaaggtttgttgtttttctttctttctttccttctagagaatttgtaagaggaaatgagcttcctcgtccaattcagtatttatgctggtcgcctgttgggagtaaattagtaagtgtttaaatttttgaagtgtttaatcatgtaaataaaagaggagccatttgattttggcctttgttaaatttccctgcctgaaagaaaaaaaaatacattcatgactattttagtggttaaaaacgggaagagttttgataaattgaaacccatctgagaattccgcaaaatctcaagctcatggatagacttctttaaatatagcagtcaaaaactttatccttatactcagccagaatgcgttgtgccagaaagctcaggaaaaatatggttttcacaagtttcccagactagcttcagacaacatgaaatttggtccaagctcttatttaaggactctaaataaagattagatctggctagctattaatattcagagatttattcgactttctgtcaatatacataattatttatttggggttaatgaaacaactaagaaaataataggctttattttttatttgattcagactttaaaattcataattgatgttaatgtgagttggttggattttttgacctattaaaatggtattttagttgtgaatcttttttgtatctttattaccactctgtttaaaggtattagaggaataattaaaacaaatatacagccttcatttaatttgttgtggaatttccccttaccaccacttttaggcatatgtctatcaaaacaatatctatttgaaacaaagaccaggagatccaccttttcaaataacatttaatggaagagaaaataaaatatttaatggaatcccagactgggtttatgaaggtaagctgtattccttttctactgctaatgctttaggcttttaatttaaaataatttcatatatttagtaagatttagatgagtagtatcttatttccatgaagggtagcattgctgaaaattgtctctaggttacgaatagggcatacgtattttaaaagaacaaattcttgttatcagaaaactggtagaattgattctcaataacatcactatgaagtggtagtaatgatttataatccaaaaaagtatatggtcctcaggtgccgcattagtacattttctcatgtttactttttctttagaaatttttttttttcaaaactaaacaactgactatgttatcttatgggaacatatttttactttctgatactctgttgattttgttctcattctaggtcatgtagaattcaggacttaattggactttaaagagacttctagtctctatctatttatctatctatctatctatctatctatctatctatctatctaaactaactgtgtttttggttttttttaactcttaggagggaaatgtaactttcaatgtttatgtcactctctagtacaattatagttctttctgttttggctatcttaggtgactaaaaaacctgaaactgccctgaaagaaaaaattaattagtgccaacatgtttattatttatttaaaaaattacatttctgattaatgtcattttctaacaaggctaattgatgtttgatatagagtgaaagggtaaactattcctcttaactcagattatgtaattttttaataaccaaagtatgacatatgtcacatccacattaaataaattatttcagttgataaatagctatttagaagtatttacaatgattttaagagtaagatgtaattatagttaatatcctagtacttatttttcagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggtatgttgaatactttcagcgatgctttcattttcctggtatcaaaatgagcaatgatttaccatgagtaaaggtgggatcaacaagtttggagagcttcttatccttgagcattgtaaggcaaacattgtactgtgctctattatttatgtggacatctgttgctttggacatctggttgtgctggatgaagtagtctttgcaaaatctattgcagaatctttgaaaaacatatggtcttgatcatggaggaaaaacaagcttaacttaattttaatttttttcatatttcatgctccaaattcagggtaaagttctgttttgcaattctctcttgaattctcatgtaaatgcaaactagaatatgaaatacaaatgctgaatttgatgttcctgtcaggtagaagcaggcatgggttatggtcctttctggatgtacaagataggttaaatttataagtctcaatttctcccttgaaggaagtagacatggtgtgaacccatagtttttggatatttaacattgtagagttgttatacattctttttattgagtttcacatctggagggaggagacctgagtgtgcagtaggagaatggtcaggtctcagtgaagaagcattatatcctaatttcaatgtaaaaatgatatcagaaaaaatggaatcaaagtacaaatttacctcttgcacaaggaatcggatgcgattttagaattcacttctttgttctttggatgctattgcttttacttgccaaggggaatattgtcgtggctggacacagtgggtccctgggtgacaaagacggttggatgtggtatacacacaagtgaattctaagcaagccaaagtggccatagagaaagaaatagaattgcagacagtgtaatatttatgttaaaagtctaaaagctgtgtaaaaactctaaaagctggagatgtttgggacaggcctcacattttgggggaacccaagatgtaaaagaactcagatctttgggaggacctatgggagcaggaagagacggggaacccagaactgaagagtgactgacactgattgggaagttgggacccacattctcatatttccttgaggcttaaggttgctaaaacatctggcgttaggtggagacgtgagaccatcccaaaaactaagggagtttctgataaacttgggagtgaaacggaggtcctaagagacgttgaacagttctttggatgaattcaaacagcactgagtcattacatactgtgcacctacatttcatgaatttaaattttattacaactccaagattctctctttctacagcaaaataacagaggaactctaagaaactgtctaggtatgagctatactgtcatgtaaattttaagcatacagtagtatagaaagcacaaaacaagaaaagtataaatctcatggatacttcatttggaggtgaaatttccctaaccttttggagatggtggcagtaataatgcaagcaggtaggagactgagcatttggtgattgtcctagaccaaatcatatggcaaaagcaaaaagtgtctttcatttatttttctttccattctcctctcaaattaatcaccctctgaactctccttggtatacagattattataattatcccaataataaaaatgagagagatctgtcttggaaatcagaaaacagaatatggttcacagaactgcattttcatatccaaaatgattttccctataaattttaaagctctctcattaggcagttaaaacacatagttttgctctggttgacacaacatttgtaattgccctgcaggtaaaattgaaatgagattccttgtcgtgttataaatagaatgtattccacagtgatatacataagattgttcgcttagaaaaatatatctaaattcctaaagtttctttaaaatgttggtgataattggaattttatgagcttatatatctcaattttcttggcaggctggagctaagaatcccgttgttcggatatttattatcgataccacttaccctgcgtatgtaggtccccaggaagtgcctgttccagcaatgatagcctcaaggtagttcatctcagatccctcccatctttctatcctatattctagtgcatttatgctgtgcatgctagaagcaaaaacaaataagtagataagagttgcacacaaagctaaagacatacaaaattaaaattgacaaataagtttccttaattggtttttcaaaattagcccttttacacatataaacgtgacctcggtgatcaatctcagtgaaacttgcctcagatgtacagatgtgaacttgggaccaacaagcagacagtaaaattcatgtacgtcctcaccctggcttctttcaagtttggctgactttatctcaagccagggtttgtggatctatgtggatcacatgtgtatgataaataagagaagataagctgaccctctccaaaaattagcatttttcacaatgaagcaggctattctcatttatctggttttagaattgtctagagatgaactgtaggcaggcagggacgtgaaggattctcacttccccagcatgatgccctatgtctgattgtctgactagtccagaatacagtgaccttcagtcagtcctctgtggtctttctggttgaagtggctaaggggaagaggtgcccaggccctgcctgttgggctttcttaacagttccccatgcctagcgggccaatcttgggtcttccccttctgcttcacgaagaagagccaagctcaaatgactgatcactaatagggaaaagaaccccatgagaggcttctactgggccttgctgagtccactcttgtataatcaaacatgactttatatttttgaattatctgctattccccaccctgatcgagtcgatgaaaacttgactcaatacatcatttttttttaaagtgtcattctccacaggtggcagatgaaatgagcataacacaaggagtccagaggcttgtttgaacagctgtaaaatactaacaataaaatagtagtagcaaacgtattctgaacactcatcatgtagcagacatagttcttaagcggtttaagcacgcggccaatctttacagcactctttaaaataggtaatattattattctgattttatataaaaatagaagagttaagtaactggcctaaaggtatataactattaggacataaagccaggatttgagcctgaggtcccttccagctttgtgatttcctttacattaattttccagaagtttgtgattcattttatgttgatgttctctacctactagttcatgctcagagtcatggaatcatcgttgccattcactgacatctagtatgtgccaaacattatttgagttatttcgcatacatcatctgtgatttattccacaagtctgaatttcaatacctgaccacagtcacagctaaaacttagcttccaacatgaatctgctgagagacaaagattatccttttatcacacttgtctctctctcaactaattataaggtgaaaaattgagtttacttgtgcaaaatgcaagtttaatgctttttttcccctctagattgatcagtagctttgacattatgttttaaattcttttctaaaatgttaaaatccagaaacggggagaaaactcacatatgaaatgatttcattagatgaagaaactctcatagatatttttttaagtaagtagagatatttaaactttctctccagagaaaggaatgttttgtcttgcttacctattaaataagtaatgtataataatgacatatatcctttagaaaacaaaaattaagaaattatttgtgtgtgaaactggtttatggttgtgactacctgtttctattttatttgcagtgattattatttcagttggctcacgtgggttactgatgaacgagtatgtttgcagtggctaaaaagagtccagaatgtttcggtcctgtctatatgtgacttcagggaagactggcagacatgggattgtccaaaggtagggtatattaagatcacagatacgtatttctgaagatcaaggcacaatagtggggactcatccatagctaacattttatgttgtaaagccaggattactgcattcaactgccaactgctttgcaaaaacacactcagtattcatgggtccatcattttattaaataaataggataccttttattttcttgaatgacgtaaaccttgaaattgaatagccaaaaagaaaaaatgggacaacatgacattgggaaaatcagatatctggttctcaattataatggtaaaccagccctttgagattataacattggcagtccaataaaaagcaaaataatgcagatgaaaccgaaacatgggtctactagataaatatatatgttttaaacaaaagaaccagagaagaaatgcaaatggggcatgttctattcaatgttaaaaattaaaaattaaaaattctaaaggaaatggatattatcaaaagaaagccagatattctgtttttttttttgtttgttttttgttttttttgtttgtttttttgttttttgttttttttttgagatggcgtctcgctctgtcgccaggctggagtgcagtggcgcaatctggactcactgcaacctccgtctcccaggttcaggcgattctcctgcctcagtctcctgagtagctgggactacaggtgtgcaccactaggcccagctaatttttttatttttagtagacacggggtttcaccatgttggccaggatggtctcaatctcttgacctcatgatccacccgcctcagcctcccaaagtactgggattacaggcatgagccactgcacccagcccaaatatttttattttaaaatcaaatgagaattactgagaatgtgacataagatattagatcaaattattctgttacctggttatcttaggacagtacccgacatgtactaattttgggcagaaaaagcctttaaaactaagcacagatttattgttgagttttaattgtttctattactcaaactcaaacataatctgttgaattaatgagtcagaaaaaccacttaaccaatgtataagctttaaaataagcctgttcttaattactcacattagaatctaagagatacaaaatattttctttaaataggatatttgatgggtacattttgtgagtcttctaattatgaagaaatgtttcattaaaacaaatatagaataatcatattttataaagtaaaaagaaaagcacagaacaaacaaagtaaatcactaaatttcatctagaaattaggacattatgttttgcaagaaaaaagtcatatgagtctgtttggaaatatatcaggactataatagtcaacattactcaggctaaaaatatttctttaaatcactactgtgtctgtgagaggctcgactaattttgatggaatatgctagaagcgtatttttatcttatatatattttgatatacaaagcaggatgattttggctttaatacagtatcataggcaaagaaaggatagactaatcttgattatgctagttaattagaaacaattggtctggttagtcagctttgggtgactatatctttaaacttttcaatgaatctagctattagggactattgctttaattttattagtttgaacataatgagatttataatttagacacaatttttagctcctaaaactaggaattttaaaactgtgattgctttgaaatctgaaattctaatttatcaaaatctgagacaactttaatccaaactgacagaacatttaagatgctcattcaatgaggtgtggaagtccctggaatataaactttagttttacagccagaaaacagtgcttaaatttagaatatcatgtttgaataaccttagaatttctaaacacctctgtttcaaaatagtttgataaatacatgcaacacattttgagatttgagaacaacagtgtcgaatttcccagaaaccatcctcatgactgatgtgtgtgtgtgtgtgcgcacacactttgaaaacaatatttaaggttaagtgacccggcaacattttattcatgctgagagtcatgccccaaacaaagaggcaaaggaaactggcaaaaatgacttgtaataaaattatattagtgagatttattaagaaatttatagttaggggaagaaacatcttttatattatgcttcctcctctgaaaagggaattaaaattaattttacttttatggctaaaaataataaatccaaaaatttcactattttttccgagtcttatcttacttgttctctctgaggcattggtctatccctgtaagagaaaatccagactgtgttcttaaaatttgttattcccctgccttttatgacagtacaccctctcatggttctccttctgcttcattgactgttcttttcctttccagctcttcttctgacttctctagaaatcttattattctccaagttaatgtatttgcaaggtaggctaactgctataacaacaacataaaaacctccaataattcagtgggttaatacagtagaaatttattgcacattcatgtaaccatccaatagtgagtccaggtaagtggggtatggtgggggttggggaatggtgcactctgcttcatatagtcattcgggaacctaggtgatggcatttctgctaactagcttccaacactattccaaagttggtcaaacctgagcatcaacatccagctgagggatggagaaaagaatgaagggtaatgtaagagaggcttttatgggctaggcctaaaagtagcatgagttatttctgttcacacaccattgactagaacttatcaaataaccacggtcaactgaaggaaggctgggaaatacatttcctgcccgtatccctagcaaaaagaggaaatggtctgctgattatcctgctgtctgtctcaactctgtacttactcactttgggtaattttaatctataccagagctttttggtatacttgccaataactctcagacttatttctgaggatttgaacttttcaatatgtgtggcttcattttctctgttctattctcactgccatgagcttgattagagtctttatcattcttttcccaaacattataatagccttccagttggtctctctgcctccttccctcttacccacttctagtatatcctctgcatcatgttcataattatatgttccaaattgtgaattggaagaccttcaaaagacttaccaaacaggagaaattgaaacttcttagggtgccataaagtttctccaaaatcttgctcataaacatgcattctccttcatcttctaactctttcctgtagtcactctccactcagccatactgaaccatctgttgggtgactaagcaggtctggtctctatgctatttattttacctagaatgttcttctttttctaattatttgtctagaaaagtctattcattcttttaggattactaaatgtttgttcagtaaatgatcaattacattatgctaaaatataacccaaaataagattcagtgcaaattttaaaattttagctacttcataggacccagaaaagaaagaatttaactcaaactccactgttttacctacaccacatcaagtggattgtgctgggcctccatagatgttgtgataattccaagtcactgcctcatggaagatttaggcataattattaaggatctgcaataaaattacccatgtatatacaacagtgctgttatatgagatttttaatattgtccttttaaactcaaatttgaagttagttgactagtaatggtatactctgatcaggcagcttaactgtttccaaatgtccagattacatttaggaaaaaaatgttctcctaatactatgttcgatgtgatcttttgtcttttagacccaggagcatatagaagaaagcagaactggatgggctggtggagtatgattttgctatctttttcttatgttttaatgaaacttgacagattctcagctatcaaaggactatttgcaatgtacccaaactggcactacttattttcatagaaaaagtagcaaggtataaaacacataggtagtgatctcttgagtgattttgtgccggacaatttcaatgtcctttttagaattctattgaaaataactcatactcctcatgtcccaattttaagtttgtcaactgaatccttggtatctataactaatattactttttaaacaaatacaatatagatcattgttcaaaaggagacaaaatccctgatctgttaaaatcatcttaataattactagccatttaaatccaaaaaaaaatacattgggtatccttgtaatttaaagacaatctacctcttattaaatttcatataacatgtactaaatttcatgtatgtatactccttccagtatttaagtaatgatttggtatcaaataggaaaatgtgtgtttgttacttgtgatcatttatatttggaagtttaggataggtttattcaataatttatgaggatttttaatgtaaaataacgcttgaggtaaatagcaattgtattttatatcgtatatgaagaataaaaattaatacaagagaataagtgaaggttggtagatgctgccacttaccacaaacacataattcaattcaattagtatttgaagaggaaaggctacttctctaagatttggacgcattatgcaatgaaaaaggaaactcttaggacctggtttaacacattagcccatgatccaaggacaaaaaaaagtcagaaatcgattctaagatgataaattatataaaaatttaaactcaaattggctgggaagaagataaactgtaaagtgataatcttcaaatattaaaaactcttcattgtacacttttctggatccagttgattttggcttacatgctgtttaatttatactctgccgttaattaaagatactgcagttttattcattaactagggaatagttactttatttattatatacaataaatgtaaattacactctgaggtaacaagataataaagataagtatgtaattcagaaaaaaaaaaaaaccctcctatttgaactcacttttcctactgtcctaaatatcacaggaaaaaaagagtagtagtattcagtgaaagaaaagtattctctaaattttatgtattcttaatcaatctgaatctcacctttcagaaaggtaattcttagatcggtatttgaaattttgaggaaaaaaaatctaccaattcactcccattacaaatatttctagtaaagaacgaagggaagaaagaatgcttgaaaatattatttagtcatgttctccaagatgatactataactagatttactatacagttatatgaataatgtacttttatatcaacaatgataccaacaaccctttcaaagacattatcttgagcatctataaacatttattttgacaatgctgcagtgttcaaaacactgaaacactttagaatgcatcttttgaaagctcatatttataagcatcttcagtatcttaataatttaaaaaatctgtattgcactaaacttgcattcccatcaaattgagctgaatgaatgaatgaattgcctgcaaagctttagaccttttttttttttttaagacggaatctcactctgttgccaggctggagtgcagtggcgcgatctcggctcactgcaagctccgcctcccgggttcaagcgattctcctgcctcagcctccttagtagccgggattgcaggcgcccaccatcacgcccagctaatttttgtatttttaatagagacggggtttcaccgtgttagccaagatggtctcaatctcttgacctcgtgatccgcctgcctcggcctcccaaagagttgggattacaggcatgagccaccgcccctggcccagctgtagaacttttttaaaaaacaccctcagtgtcaggagatcgagaccatcctggctaacactgtgaaaccccgtctactaaaaatacaaaaaaattagccgggcatggtggcgggcgcctgtagtcccagctactcaggaggctgaggcaagagaatggcgtgaacctgggaggcggagcttgcagtgagcagagatcgcgccactgcactctggcttgggcgacagagcgagactctgtctcaaaaaaaaaaaaaatgccctcagtggaggcacatatgtctcctttgagagaagatgaggtccttacattaaatcaaaaatcattcccaactaaatggcatgagccaccgcgcccagcccagctgtagaacttttttaaaaaaataccctcagttgaggcacatacctctcctttgggagaagatgaggtccttatattaaatcaaaagtcattcccaactaaatctagagagtaaatgagttacttatctgggacatgtgattttgtgccagatcattttaatattcatagttttataattctattgggaacaacttatttttatgtactaatctcaatctcttaacatgtattcagtagtcagtaaaacttctaaattccagaagagaaattatcaaaaattatttgcaatgattgttttcaagagtcagtttacactctcccaaggcacctgctttaaatggattattaattagcttttgtaagttctgacatgccttttgaaaaagtctggccacaattttatatagccataactcatatttcataatatattgtatatttaatagcttcctttgacctataatgaaactagtgacttaataaaattaaccagaaaaaactagaaatgtgtatgtatttcagacattcattcaagtaacatttatagggtctttagataaagtatacaagctagtcgtattagtataaattttaaaagctatttttgatatatggtttcttttgagtttgtcagagatatttaaaatggaaaaagactctcagaaatcacctatgaattagaaaagggagattaaatgacttgctaaaagtaaatgatctgttagttgcaggcacagaactagaaaataggtttcctgcgaactttacttaccacttttttcctccagcactgacacatccagtcctttaagttaggattaataacctaacttcttttttggtattaatcagaaaaaataacaaaattgcttttaaaacatgtttgttttgttttgctctgtagaggacagaaaagtgaagagttaatcaaaggaaactagtgcttcattccttaaatttggagaacaagcactgaaggcatcatgcttagactgcaaagtcacatcaattattttaaaataatttaagcaacacactacaccgtctttaggcggctggcaaaatcccttggttcaaaaaaaaatgaattttcttcttgatgcttgaaagcttgtgcttttgaccgaatgtgcactcagtttagggcatccgctttaggtcatttaacagctatggcaagatattacatggcggtaaataatgtcaccttccaggtaaaatattttaaatggctttgcacttcatatagagatgagtaatctatcagaaaatctaagcataataaagaaaccagtgtaagggcctcattaaaactgaggacaacttggtgtgggtgcttatctagggtccaggtgtgacttgcaagaagcacttaactcagaacactttatgggcctaactctagcaaaatgaatgtggacatatttcacactctctttacctcacccaagttacctgttcatctaaagcagctgtcttcaatgtgtgcaatgaaaggggagaagataacagtctttgcttatcagaaatattcttcctgtttgcctgattctaatatcttaaaacataacactctgccctgttttttttattttcaataaaaatgagatgctaagttttaatctagaacaaagccctttaaaaagtcccttatctaactgactgcagcttgcagacaatttttctttttactgtggtaccttaaaaatgttgtgattcaggttttattttagtgattaaggcaatgtagaaccaatttttgtgtttcacaatgaggaataagaaggagacataggccgggtacagtggctcacaccttaatcctagcactttgggaggccaaggcgggtggatcacctgaggtcaggagttcgagaccagcctgaccaacatggtgaaacctcatctctactaaaaatataaaactagccgggggtggtggtgcatgcctgtaatcccagctacttgggaggctgaggcaggagaatcacttgtgcccgggcgacagaggttgtgatgagccaagaccacgctatttgcactccagcctgggcaacaagaacgaaactccataaaaaataaaaaaataaaaaaaagggagacattataagaggttactattaagtatgtatattatcatttagctaagagggtgaaacttcattctgaaatgaaagaaggattttgtcaaagacttatagtaataaaattttacccacaaaattgttaatcagagtttcctattcctgggcctcttggttattaatttcacttggaagcattagtctcaatatgcaaaatcctaccaaggttggtcttattattaatgaaactaatgtaggaatcatgtagaggcagctgtttctcattattccattttttcattttaaagaaaactttagacaagttaaaagtagtagagtttatttgaacaaaaacaattcataaatcaggcagcactcagaaccagaagatgttctgagagttctgcccagcaatgtgagcagccaccttttataggccaaacgcagaagcaaggtagataaaccactggttggctacactaggagtcggcctcattgggtcatagagtgatgaggcatctaccttatttggatgtggtgtgatatgatggctgcctatgattggctgaaacccttttgctggactcgtagttaggttttggtttgtatatgtactaaggttgtgtttcattatataggaactcaaattatagagacagcctcaggttaatggccttctgcttatttgatttgactgatttattgattctaggtaaatgtggtgagacttagggcactgtattagtttcctagggctgctataagttatggcaaactttgtggcttaaaacaacagaaatttattctctcgccattctgaaagctggaagtcggaaatcatggtgtcagcagagtcgtgctccctatgaagactcaagagaagaatcctccctgtctcttccagcttctcatggctcctagaaatccctgagcattgtttgtttaacagccgtatcactttaatccctgcttctgtcttcacatagtcttcttccctgtatctctcagtgtctctttgtgtcctccccacctatgaggacagcagtcattagagttagggccctctttaatacacatctttactttctcccttgattacatctgcgaagaccccatttccaaaagggtcacattctgtggctccacgtggacatgaatttttggggaactcagtccactaaagacatcacatcaaattagttgcattttaatgtcacacagttataattatttcatgagaaataatgatcggttgcatcaaaaaggcatcaaatatgaggtcatcattaaaggagcctacagtgacttcctccactcttttcttgctctacaatgggtctcacattaggtctcaccaggataggtctttgtagctaaaagatactagtagctttttgttgttgttgcttgatgcagttctcatgtggagggacaccaactagagagagctggctttaaatctgctctgcatgatgtagagtagctctttcctcatggaattcttttaatgatttgccatcatttcaccaaacaatgaccaatggggaaaaaaagtcagcaaatgtaagacaagtcaattaattttttaatgggaaaaatacgaaactattttctaaaatttggtgtaagaacatttgcagaatttcagttatatctatcatttagaatgttgaggtgtggcttgttctgtttcaaggcacactaaagagcctagcgtaccttaggtagttaattggttcccagcaagttctgcatggggttacctgttagaggtcaaaccagggcattgaggtcagtctgcagtcggcttcctgtgctcaaggagtaaacctcccccaacataggcaacttttttctctcaagtccagaagcccctcctcaccattaaaaactgtggctagcaagccatgatcttttcatcttttaaatgtcttcattgaatcattgatattgcatgcttggcatcagacaaatcaagaattaaaagaggtatagtcaatgagcatcagactttggtcaaatacctcattttaggtttggtttcgttaaatatcaacatgcgtttgtgattcttttaactccgtttcaaaccacataaccttcgacttaggaatttaatcaaattaagcattgtaaatccaggggtatgcagtagcagccaagcagtggtcggaatgttcaagtgcctgtcagaatcactacataagctgtttatctgaaagaatgggatttcagatgtgaatgacagaagacagaggaggcgagacattctgattcctataactatgaccatacgtcgcttttatatattaaaacaaaatctatggttttagtttggtttgtcttttgaacataacctctgctaatttttttcagttctttgtttcaacaccagttttcagctatgatgccatttcgtactacaaaatatttagtgacaaggatggctacaaacatattcactatatcaaagacactgtggtacgttccttccaagatttatctcctcaaagatcattcacttgaggttgctcaaataacgctcagtttcacacaacataatgtttgtctttttataggaaaatgctattcaaattacaagtggcaagtgggaggccataaatatattcagagtaacacaggattcactgtaagtattgcacgaagcacgatgcaccattcaggttttaattggcaccgatggacataaatgatactgtaccaactctcttagttaagaagatttttgttactctataattcagacagttgtgagttcatttacgattgcacatttttaataactagtcaaaatgatttccttttggaaaaaagccttcagtaggtgataatttctcttctgatatttggtaggcaattatataaaaagtatacaaattttaagataataactcgttctaaaggcaaattttacccttcagcacttgaagcaatgaacaagtttcaaaattccctggagggcagcccatagatatggagcatcttttgtagtgattcattgttatataagtttaacattattatttataacaactgtcatttatggagcaattactgtgtaccagatactgtaaagtagaaatgattatcctaagtttgcagatgagaaaagggatactcataaaatttaattaacttggccaaaggccacagttagttagtagcagactaggattcaatcctaaagcattaaatctaaggctgttattatcaacctgaacatgggactgctttcttacagttctttcctcttcagtccattctgcatactatttaaataaaccatcttcaaataatcatttatgataaaactgatctaatcacaaaagtatactttctttctataatttctaatacagtctgcatctagggatcctctcttagtgctgaaaatctgccagctattagctgaaattctaggagcaaaaagatgaggaccattagtccaggctcatggaataaaagcctgtcggaaatctattacgaatctttggaattctttgaatggagggcaaaggtgtttggactttctttggagcagtcattaaaatcactagagtaaaggggagtgacatgttcaaaacaggtgggagattcatttcaggcatgatgtgttgaggaggtggggaaaaactgaaggcggaggtccctagctctggtcataagaggcctgaactagaagagtggcaatgtaatagatgctctgaggacaaaactgcacctccttctttgtttgcgtccttagcacttagcacagcgcctgacacagagtagttcaatacacattctctaaatggaagagagaacagagtagatggtaaaaacagatggaggctaaacaggactggtgaagggagactgtcaggaaaaagagagagaaaagagacaaactggacctggctgcaagggttagagagggagacaatcagggaagcttccacataacaaggaagtggccgggggctgttggggaaagatcctgagtcctttattcacagatagttttagatgatgtggggagggcaaggaaaagggatgatgggagtcatgcaaagttataaagatgcatagaaaggtgttagtttaggaaataaatttcgtgctaaaaacgagttgtattccattgcttttttcttctacttggtgctaataagattttcatgatacctgggacatagtgagaactggactgcctaattgtcctaagaattcaaccaaacttcggttatcccaaacttttcacattcgtgggggaaaaataatgacagtttctgatactcagatgaacatacagcagagtgaaaaccatgtcatgaaactgaatttcccacttcatggctattttaagacaggttggaatcagaacctggatacttaagtaaaagcatgttctcttatgacttcttttgcatctggctaaattgacatgccctgtgtgtggtatccagtgtgatatcctcaatttgtagcaaacccatctattgacagggcttctataaaataaattcaaggtgtgttcagaatcccagataaaattaaataacgtagtacagagaaacaggtaaactgttcttaatcataaaaaagtcaaggtatgatattaataaaatttcctagttgattttgcatgactcataatgaatcagaatataatataataaaaacagtattcagcataatttaatcttcctctgatgatttggaagccaggataaatgtgcaaagttcagttatgaacagcagttatttgtgtacttaagcaccaattaattgtttcattagataaatgcttacctatattgatactatattagtattaatactaacaatcctattatcacttatttatcaatacttaataatttctgaatatattttcaggttttattctagcaatgaatttgaagaataccctggaagaagaaacatctacaggtaatttatgtccattcattctggtatttctatgtgttgatattttaatataaaaagtcatcatccccaggagatgcaaaatatttttaaagtatggcacataataaaaatcatttatgaatttagagaaaacatttggagaatgggtataaaaaacacacactcgcaaaatgttagccaattcaaaatatttgttttcaaaaggaaaaaacaagaaattctaatgtttacatgaatactaaatactattctggtactaaacactttaagtacatatagaggtaattgaggacatttctttatttaacacacatttgttgagtacctagtatgaaccaggcattgtttttattctgataatatgtgatttccttcactctgtctcactttggcaccacccatgtctgttcctatattagtgtttacattcagtccagttgttcccttgaggactgaaatggtgccctcgacagccctttctgggtgtggaaaggaaagcaatagttactacatgctggtagtctattatatgccaggcaccttttaatctgacttacatatgtaatttcatttaattccaacagcactatgaagcaggtaatgttgttggagccaggatttgtacctgggcatgctgatctctgagcccttgctgttaactgcctccctaatgtttccaaagttccacccgaatagggacatatgttaacatactaaccctaacagaggaaataagaaattttccctctgtcatattcccctgttccctcctggatcgtgatcttactttatgtgttaaaaagaatcactctctgggtccagcagtgtctctggcctggccattctctgccatgcccaaggcaaagtcagggacagcagcagaagaaactcacaggttggcacaggagaccctgacaggcaccatggggtcctgacctgtccagaccctggcatgacatcctctaaactcctccacatggaattctgattctgagggtgtcggataaggtccctgagtctgtgtattgaagccaatcaccctgctcaggaaccacacagatgactctgcagggtgagtgtagggccaatgtgggcagtgcagattggcttgtgtagactgtgtggagggggcattcccagcctgagcttccatgagcccaaatttcctgctgaaaggaatgaaggcgcagaaggcacttagaactgtaatttcttatttccaatttgcttattattaatttccccatcctgatatatggtaagaacagggactctttcttggtgatactgccagattttcttgctatgttttgccatggggtggaaacagagagatagaaagacgaagaactatgcattcccaagcactttgtgaagtgtaagagatgaatggttatgggaagtgactacctcactgccatttgaggtgttagatatttctcccactccagggaaaccacatagggcttcttcctcaaggtttgtgcctattaaggcaaagatggatcaaaaatcaagctacatcaaaatactgtgattcccctttactttaaatgatattggcaacagaaaaatagagtgggtttgatgtcaatatgtaaattagtcactttacatggagtagtacattttgataaatacaatctcaaaatttcagcttagttttattttcatttccctctagaattagcattggaagctatcctccaagcaagaagtgtgttacttgccatctaaggaaagaaaggtgccaatattacacagcaagtttcagcgactacgccaagtactatgcacttgtctgctacggtaggtaacggacctcacataagcacagcatccccacaggcatggttgtcatcctcctcacctctatctgatcaattcgtagctacagttaatgagcatttgttatatgtaagaatcttatcttgtgcttatagaaaaaaaattaaaggaggataggaaggtattattatctttacttttttgcaactgaggaaacagagtttggggaaacagggtaacttgccaaaaggtccgcactgcagctaagcatcagccaggactgaagcaagtgctgttgcctctaagtcccatgctcttaaccaccacattccacagcactaagactgcgtgcagaggcaacccgtcagctaggcttcgcaagacctgagcaatcgcagaaaaagaaatgttctctgataaaatggttgctggacttttggccccctttgcccagagaaatatcaaactctcacatccaggggaatgaacaacggcttggaaacccgagatgcccacgtcggaggtcaatcattcctcaaactgtagtttcaatggggatagaagatgaacctttgtcctttctaaatttggaaaacatcgtaagtaatgcatcaaatataacaaaccataagtaataaaatctacaaaacatttcctggttgtttaggacatacgtacatttaatgaataatttctacccatataattttattttatgtgcaaccttttatctatatcttgagcatgtggcaaacaataaaacaggttaggttaagaggccaatatagatcaatgaaatatattggggtctaagtagtcattatggagatttggaaaactgactcgggaatctctaattctccaatctctgtcacctccctccagcttattcctaatttggggctgagagttagagtacttggaaaaaggtattacaaaatgtatgctaccctcctgttttggtatcagctcccctggtaggggtatgtggcttaaagaacaaatggaatctttgactgcttgatatccctattcagttgcagaggccctgaagatctcataaagggaaccagaatgaaggctaccctaacatgccacttccctcagtccagccacctggccccggaacctgagctgcagctcatttgactacgtggacatgaataagcattttaacccaaattctcccttgaagatagcacaaagatgtcagtttgaaataacactttcataaaatgtttggtcaatggcatctttgaaatgtcaagagattgttgttcatgaccatatgtgctgattgtggttactggcatttaaaggtatacattaaatgtccctctcaagttatatgcaacatttttcaacctaagctatatatttatgcaacatagattgaaagcttacatgccaggcacatagtatcattgagctaactatactgagtcatctgtttatctatctttttctcctggaaggcaggggctacattcttttaatctttgtatttctaagaccaagaagaatgtctaatacataatagacattcaatgtttgtggaattaacatagatatgaggtaacattgctgtcccttgcagtctaccaaaaaagctggttttatgctgatgcatttgctgatgaatgcatcagtgacactacatttacatccatgcatttatatatgtatgcattctgcatttgtataatgctttaatgtttttggaaggcactatctcacattttgtattacgttgtcttcctaatatctacttgagcctgtgtgcccactgaagagatgaagaataaaagcctaagctctcatcaaaagttggtggaagagtcctttctagaacttgaatcgcctgacttttccaccactcactcttcctcggcagtatggtaatttcccatttagcaaaaaatatactgtgtgggaagcatcacatcgtctggtgtttttgaagctccagtaaaatgagtcactgcgccatcataggaaaaaccttgaaatctttcccttcaggactaagagtaaagcctcattatgtaacttcaaactctcccgagggatggttagtaagtcccagatgataggatcaaaacaggtaaaattatatcgatgatgctgatggaacaaaagcatgcaaggtaggcttctcaaggtctgatgtttaggccactaggatcacaccttcatccagtgtcaaatacagtcagtcatttatctgtctcattttttaggcaaaaaaaaaaaaaaaaagatatgcaagtttccattattgaaactacacactcatgaaaattccacagggtagatgctggttgattagagtctagaaatttatattcatgtctcatttctttgcatgctggctgaaaatgtaatatgtgtatttgtcctttttcataactttgatgaaaaactggccaaaatgaaaatctctgcaataacattctataaaattctgagattccacacaggaagttttgagtcttgagggagcctaataaatactatcctttttcctcatttccaaattagactaggattcaggcaggaaaccaaaatattatctttacttgtaaaggctagttggaaaatgtggtaaagctgtgtggtttcccaatattgcatcccagaattatacttacacaaatataggtatgacagggcagccaccatcttcctccatggggcttggcactggcacaggtacagcactgaggaacggatcaaaagcatgttctctgcaggcagaaagatgccagaagaaagacagaaggagctactcatacccaagtcttctcagaactctacaaaaaaatcatttctcctctgcttagcatgagccaagcatgctactttaatgggttccctccacatagaaggtagcatcctcaccctatctggggggggtggggtggcaaatgaacatgtacctccaacctccacatttgttgcttttaacctactgactgagcaaatttatgcttaaaaggtatatttccagacattatttgatcctgtatttcaaataaggaataatttgtttttaattatttgtagatcttgctaaaaatggccagtttctgcatagagaagctgaatgcatgttggaatatgtagtggacactggtgtgccagccagatcccttcatccccatcttcaggactgactcagtcatttcttcagctactgagagtgctagtggctgacaacttacagctgagcacgccccccaaccctacctccccccaacccgggagaagagaagagttttctcttctcccgggccacatccaatgctaggggggtaaagctccatgctagggggtaaaagtcagccttgttgccccattggagacaactctacaagctatctaggggtctctatgtgacagcaatgacatccacctttcttccctgccctattcttcctccctttcccccacaagtgtgcatcccaataccacccccacaaacctccttccaagactgcttcccagggacctgccacagcatattttaaaaggaagtatatcccatgtagatttctgcatgtattattccatggaaaaaatactcgattttacccatctaaataagctgaacattgaaccaagaagccagattgaaaatacaatctgggtactggtgaaggttgtttgataataaagacacagccaaaactggcccaatacagcatgacatcttcccagaaggccatttgtcactgcagcttctggggtgcatttgtgtaattcgatattaatgattcaggattagtcttggaaaagtgcacaggcaaggcttctttcacagctgttctttgagtgggctttgatctcatgtattgtttaaatgtgacttagaagtgatgttttcttctctatggggaaaaaaatagctgcagaattaaaagatgtactggcacttgacactaatcacaaatactaccagcagcccgtcttgaaaaggaattgttattttaacactaagccatgattttgttttcatcctgcccacaggcccaggcatccccatttccacccttcatgatggacgcactgatcaaggtactctctcttgcatgttatctatcttgtctcaagtaatcctgaactctctgtgatgctccgctagagaaagcaggacagtttctccacaccaccaaggggaactgcgatggaatcctgttatcacatcacttattattgcacagatttggatattatttgggtcaaatatgatcctatgaaatttaataattctttgcagtgagaacctggcataacacagatagatgagttatatcagagggtttaattctatcttctccaatcagccttataaaaagcacagattagaattgggaaaaaacttagcacattcagtacgatcttccttccaaaatagaatccctagggtgtttttttttttttttcctcagggattgggggtagggggttgtttgcatgtgtgtgtgtgtgtgtgtgtgtgtgtggtaacatcccaaactggaaacaaattctacatgtaacttcttcagttcagagcagagggtgactgtcacaccagctttttttgttttaacagctacatcacattgttgccacgtatgaacgccctgaaatttggaggctctttccattttcaactgctatcaagacagtcctcctctatcctgtatttgtgatattggttcaaaatagttcacatctcaaagacgaccagttttgctcgcagcagagttgtccaagtatctaattgctctgatactcttcctgctggctctccattatcttttgatttactattaaacaacattatagggttggagttgttggaggccagggacaatgaggcagatgccactttcttcagcactcatgcctgcgattgtccctggacagctttggtgacaagtcaggagactgccttgggattactttggctgtcagctcaatggctcaagtgtaggtgaaaataacaagaaaaaaaaacaaaaacagaatcaatttgggaactctggacaggacaggcattgtgataatgggactgtgaatttagtttcattttaaggttttttttgaaaccttgtgtctaataaggaaatacttttaagaataacagattatatttcttaatttgaccatctcagtcagtgagattgatttcaagtcgacaaggttttgttttaatagaaatcactattagcatataattctcatcttttcaagactgcaaaataatagccagcctttctttttccatagcatgaatttctactttcttgagatccttgttagattcctggcagatctgtgtactgattttgaagctctattacttcactcactatttacaaaataggtacgccctcagatatgtcagatgtaattaaataagtaccaacagaaaaaatcaaattctttcagagcacagttccacaacttacctaatgtgaaattattctcctatgttcattataaatacggattcccaggacttccccaaagattttaatgtggtacgtcttaagttatgcttggaaatttgtatttttaatagtaaatgttcagctgctgttttggagcagatgctgtgtctcttatttacattgcatttggtgtcagcaatctcattcaacaaatatttatggaacttctaccacattaagccaggcactgtgctaggtcctggatctatctataaaataaagagatactgggttagacgatgtccttgtcctcaaggaggttatagatgagtggggaatatgaacatggaaatgggcagttacaatacagtggggtaagttctaagataggggaactgcagaaagcctggaagcacacaggaagagcagctcatctgggtttggggggttaggaaatgtttactaatgataactaccacaaacccgagactcatgaatatgaagtaggtgataatgctatttggatgattggcttgatactaatggttttttactgaaatagaggagtcattgtggagatcaggttttgggaggatgatgaattttgttttaaatgttgagcttgaggcactgatgagatacctaagtggagctgctctgtagtgacatcattttggatctggatgccagaagatcagtttggactgaagataacgatgaaagtcattaatttatggagcatgtggtaactcaggccttggaagtggatgatatgagccgagtggacaaaacaagagtggatagggttagtggggagtgagcctacaacgaaactgtgtggaacatcaacacttgagaaatggaaggagaaaaatgagcctggagaggagaccaagaaggaacagaaggagggggataaacttgagagtatggtattgtggaaagcaggggaggagaagagctcaaggaggaggaaataatcagctgtaccagatgctgcagagagtgagggaaggcttgctctaaattaatggacttagcaagagggagattgacattagcttggtcagagccatttcagtggagcagtggaggtggaagccagattaaagcaaaattaggtctgcataagtaagtttaaaaagacagtatttccagttggtctgagtgtgatgagaagaggggattggtgggacgatggctgtaaaagattgatgtaggtttaattttgtttttgaaagaggataaacatttgagtatgagtggatgtgatagaggaaatcagcagaaaaaatggaaggaaaacatagaggagggaggtccctaagaagttgagagaggatgaagatcagagacaggtaaatggattaatctcttgtgatgtgatgagacagaataaagaaattgttggtagaattgcatgaaagttggaacacagtgaagtggcttcattttccctgcaaagagggggaagtttatttgaaaacagcaggtagtacttgaaccagattccgtgggaatccagaagagctggaattgtcagggagggcccagttaaagcttagttgctttgggcacagaaaatgctcattaaaattaatcaattcatgacatgcccttattaatcagcctttctatggattctccccataatttaatcagcaatatgggattttaaaacgattcaaacagaaatcaagtataagatccatcattgttacaactcaatttcatcacaatgaagagcctacaaatacatgtggaagcagatactacatgccagatactaggtgctgtgtgtgcttggtactgagtatacggtgatgggagaaagaaagtaaaatgtgtggtctgacctgtactccctttgctaggttatgggtttttaatgcacagccttattgagaagcccagctgccacctctcactcttgccactgccccctgtgagattctgtttgtgctccaagaggtggaaaaattgcccagtcactatccataaaaaatgcaactctgaaccataaaagcatttagattaaattttgcatttcattaagtcatgtatcagaaaagttcgatagctaagaatagaatgaagaaggagtcaaagccataaccacgtgacaacacccggatactgtctcccacttccagtgcttgtggtaaaataatacaaatagtaatcagtcaatcaatcagccagtctgtgtgcttgatccacacaggagtctctttatcgatggcagctccacaaacagaatccacgcaagaatgaagggttggctctactgtacttagaattgtgtattcattttacttttttggttttaacttttaaggtgtcacaagtatctcttccctgatggattacccaaagttaatgtgaaattcattatgtatcttatacaaatcatacccaaaacatctatacttgtgctaagattaaatataacatgttggagcaaaatgccatgagtatatgaaaactgagagttacaagagaagtggtttggcaaacttagtatagttacaagcactcctccagctctctctcttcagagaccactctttcaaaaaggcataaaaacaagaagtccatggacatcttttgtagcaatagatatacctcacaagtcatccagcccctcttttaaaatataaagctaaaactccacatattatttgaaaggctaacttttctctagaagatgctgatatcaatcccaggttcaaaataaggtctataaaaagcccgctattgctagaaacctatttgcatgtattaccaatctacagattcaagagatcaatggatttgttccttttcaaatattaaggtgtgaaatataactgagctcaagtataaatagcaggttttagattttttaaagatttaagacagagaaagagcatagtgtgtgtttgtgtgtgtgtgtgtgtgtatacacaatatatattgttgtcttactcagatatatcactaccttgagagaaaggatctgaaagaatagcatacaactaattcaattaccaaaaacaggagaatatcctttattaatcaagtacaacaggaaagcgtaagaaagaaagattttaaaaatcacagaaataatattcaactttacacattaaaaattaaaaagcaaattttggaaatacacaattaattattttaaatttgaggccccagcaatccagattgtatttccactacatttttttaagtatatgataaaagaaatatcacctgcttaggtcatggctttaaacttaaaaacaaaacagctttagatgggaaaacgtgattgaataaacattgttgctcctgtgatttttgctcaaaatgtgttctgatgctattagaattagttaaagacacagtgattaaaatgtgatgtccttcataagcttatatctgctctaaaggctggattataaatacatttgtcaatatgaaacttcacttcttaagcgagcttttcacagtttcagtttcacccatattgaagtcaaaggaattttaaggaaaaaaattgtttatggaaatactattgattaaaaatgaacatttaacatttttcagaaattaaaatcctggaagaaaacaaggaattggaaaatgctttgaaaaatatccagctgcctaaagaggaaattaagaaacttgaagtagatgaaattagtaagatcttttaaaaacttctcatttatagatatgaagctgagcaattttcttgcttttaaaaaagcatagagtgaatgttacagtgcaaattctgcgatctattaatattacaggatttcatttaaaataatcttactgtaacaattcttagttttactaaatatctttaatatgtttgtgaacatggttgatcttataccttttcaatagcttcctttccacactgacgtttttttaaaaagagcttatctaattttaattttaaaatgtatccttctagaacaaatagtaattcaaggtttctagtcatttatacatttaacacaaaaatttaataaaagttacttttaaatatatcctaaattcttaacaatttagtagttgaaaattgattttattgcatatattgttattaaagacaataaatgatgctactagtgctgtggaatgaagatttttctcctttttttttcctcaaagctttatggtacaagatgattcttcctcctcaatttgacagatcaaagaagtatcccttgctaattcaagtgtatgtaatattttctttggtgtaaaaactatttactgaaaatatgttagatgaaatacacatttatgaaatagaatgaaccattctccttggtaacagtgcctattgatgctgtttttatatattcttcaactaatcagagtaaataaaatatggctatttaaaatagattgaggaagttttgagattttagctggacttgcttgcatttgctgtgttaaatttcctttgtatggcaatttgccgaatgtgtcagaatacctttgagatagagaaattgcagccaaaaaaaatattgtaaatcatcttcacatcttatgtaattgagttagaaatgaaataaaggaatagaatacatttagtggaatcacacccaaaatcagtgctgacctacacacaatttgcttaaaacaccatgcattttttttagtatatcttatgtctgcttttagcaagtggcaggtggcagaaatgacgaaaaacccacacatattccttgaaatgccaaccctagatgtatccgttttggggaataggagaggctgtgtcccaaaacaacttccttgtagcaaacctctgggaaaagcctggactcctcttgattttgagccagttctaacgccattgttattttatcacataattgtactggaattactagaagcaatgcttggccaggatgggaaattgagttttacaaagcaatcaaagattcttgttttttttgtttttcctttaggtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcaggtgagcaccttctcattcaagcagatgtcaacatgcaacaaaagtcatgcaatcactattgtgtttctccattcttcatgacttaattcttttgtttttctttttctaacttggtctaaaactatcactaatcttaaagaggtatttctgccaaatatgaaaaaaaagtatttatggaagagagctaacgaatgtgtagaaaaatgatttcaagtgctaaatgacaaataccctcttttgatgtgttgtcattcattccaatatcttttaatttagttccatttcttacaattgatagaaaaactttgaactctaatgatgatcttcaggaaaacaaatatctgaatttgctcaagcattgtggattctgcctcttaagtttagaaatggctcaagattttatcagcactacatacttttagatgttaggccttcatttgtggttaaactagctatcagtcggccatggtagcatgaaagtgtttgcctattttcgtttgacaaaaacatagtcattttacacaacagaaattatattatgaaaacatttagggaaaacacagattcataatctgatttcttcttctcaactatatatattttcaaccatgatttatggttcaatcatcttttcctgaaacaggactatataggcaccaaaattgtggcttttttagaaacactgtgcaaataattcttgccatgtctctatagttgttgggtttcattattatgttatgaataggtgaagctaagaataatagttcgtaaatttaaaaggtagcaggctaattgcactctcagtcgtgaatatatgtgcatgtgaggagctaaaggtcaaacactgtttcacaggcaaagagaaatttagaaatttctttagtacattttttatccccttaatgttattaatacttaatgatttcactgcaacatgcagaataaagattgtatttaacagcctggagccaggcctagacaacacttttctatatttttgtctctaatcaaagcagaagagtttagactcacaagtaattatttttaaaaatagaaaggtgtccataacccatacattttcaagtgcttctttcattctgggaacaaacaaaatgttcaattaaaaaaaaagtctatctgagtacaatacgacaaattagtaagtaggttactaacactaaaatctgtagctatcactcaatttctggaagctccctctttcttatggactcagacttttggcctttcaactattcacttgtttggaggagatagttcaaagaaaaaccccactgagacatcagtttctcctataaagactgtagtgtgccttaatgagaatattaacgccatcctcagaattatagcttaattccatgggggactttttttagcaccatggctcacatctatttgcatttaaaccatgcaataaacatcaaggacatgtaatcctgggactacatgaaaacagagatgaaaattatgcctcatttataatttatttagatcttttccaaagctgaaagagctaaaaagttgtttattttggcattggttaaaaaatcattgtcttcatcatcgtcatcatcgcaacccaccagtaacaacaaaatatgtcattatacttttatgggtttatttaccaccacagatgcaccatttgaacctaagaagaaatagcttcttaatattttccttggatgaaatgaaaatttaaacctgagccatgatattcaacatacaggacacaaagccataaattctaccttctactgattttcgcagtaataccaatttgtctttggcatttagaattgaagatggactagaggtggtaattggatacctctactttctctgagccatttttaggatggaatctgaagaaagtcttcctgctatagggaaaacatatgttcacccatatactcagccattcaacaacatagcaaatattaatattaatacatgccattgcagtacactctgtataaaattgtgcaatttgcaatttctgttttcagtgtgcttgcaatctggtaaaagtaaataagccacctacactctgaagggacaaaattgttgttagatcaaacttttagtaaacaccccagagaatatgggcagctgcgtggccccatgcaaagtctataaacaacagtggagatgacagccagagctggccttggtttttgcttgaaattgttcttatgcaactgtgaaaaggaagccttataatccaatgctttgagaaaacaaaagctaaataggatagactcagaataataatcctagggatgattcaatatacctcctaaggaagtgttttaggaatcagacactaaatgcctgacctggggaattgagattataatacttctactgtagcccttaaattgcctctcagagaataaaatttctcccctgaatcttttacaaaatgcaatctggctaagcttttgacataccaaaagaaaatcgtacgtacatatttggaaacaacagtaaatactactcaaggccaacaaatagatttaaacaaagcaaagtaggggaaaaaaagaagtcatatcgtcttataacttctatctatgtgaaagaaaatgcgtcagaagagatgtttgtgaagctacagacataagggagactggaacccgctttctttaggtgtctcttcaaatggtacatatgtgagacccttaaaactttggtggttataagggcaactttgctcatttggagtatatttggcacttttgatattcccacaactttaaaatttgatataacttgagggttagtggtggggaagaattacatgagatgaaattttctatcatgcttttatagatggcatgaataagaatatttctaaaataggataataattgatcgctatgattccaagtttaccttggccttgggccaactttatgagagaaaagttataaagttcaatccagactccaagattagaaaaacatccttttcatttgttactgacattttgttgatataaccacattatcaacagtatttacaaccctctgggaaactcttcagaatttgaaggtgaattttatagtgcctcgttggtctggaagtgctaagattatctagctaacctttaacctagtcctgcaacattcagtgttaatgtttcatttccatgttaactattgcactatcatttcctgtaaatgtcatcctttcagaataaacctaggaagagagctgttaagagtttcctgttcccatgtatcacctgttatttgctctttaccccagatgtaaggtagtatgattgttggcttctgggagatcataaaatcacaattgattttatggctcagaactgtcttcaagcaccagtagcaaggttattttttttttaacaagaaagtgaaccttctcttctttcatttgatttttgaagtttgtgcaagaattgctcaagtgtcacaatttgctgcacattttctcctctataccatgagcagcatccacacaggtagcaactttccatttgctactgcatttcttctctgtagactcctcagtgtccttcagaatcagtgtcgggtaagctgtgactaacggggcctcttcccatctgtttgtcccgggcatttgcccaacttctctatgcatcggaaacgtgcatgttgaatcaatgcaagagcgaaatggttgctgtgaaatccctttaagaaactacattattcaagtaatgaaaatcactcagttctacttccagagaagaactgatgagaaaggtctttaacagtaattgaatggaacaagaagatagtatttttctgggactgtgtgtctcctttggtcttctcccctccttcctgtcactgttgcatacggtggtttctgcaatggctgatccagtttgccacattccactccggctgctgtttctctgagattttcttgagtttttatcaaaccaatccccagataaatcacaattgctctagaacaccagccctcagggtaacatgttagaaattagcttgaaaccctcaattttgagggtttcccatgactttttaaacgttgtccttcagtgtagccccttgctcctaaggaaagatttattttcaaaatagttttcaaatggatgctcttctgtgatggagctaaaaaggaattatttccatattaattaatgatgaattgaggatgcctaagtatgaaatggaagacggttgtatatttctctcaaatgtctctgaaaatgaagtgatgattatcattaacaacaatgtgataagaaattaatataatttaaagcagcaagaacaaaatccttctttccgatagctggggatttggaaacttggtcctgctcaatgctgctgctacttcttgtgtcactatgaacccatcttttggcctctctgagtctcaggtacacccccacgattagactacaaggccttataccgctctgacattaccagtcaataactgtacaacagagtctaagggaatccttttatctctttgcagaaaacatgataatattattttaattatttgtaaatgcaggcttattatgcatgtgtttaaacaaagaaaatgtagcgtttctattaaatctttggattaaatatggttgtgtaatgaagtaaatgtcatgtgtatacacttaacattctaaaaatttgtttcttttactttagaaaattcatagaaatgggtttcattgatgaaaaaagaatagccatatggggctgggtgagttgttttatattttttatgcctgttattcagttcagcaatcatcattttctgaagtgttcgtttagaaggtttctcatgaaatgttaatgttgcaacataatttaatctgtcaatggaaacagtcagtttggataatctgggcttcaaaatggtggttttaagtgtttgttcctttttttcaatcttttcttccctgtacagtaagttaactcataattgacatatttttacttaagtctttgttactagattatagcatactgaactgaaccatatccgtcagagggtgaattgcctttgttattttaaagttaaatatataccttttattttatatttctattttataaaaattggagctacaatttgaaatcacagggactttcagaatataaattccttagtacctagcctagcagtcatatcttagctgtttaattttgtttctttttatttggcaatcaaattattttagtagaccgaatgttcgtttgtgctaactggagaaaagtactatttagtttcctaattataggtcatggagataacttctcacacttgaggagctattttgaaaatacacatttaaagaaagtggcaaaacttatcgtttatgggcaatgaagtcactggaaagtttagagaaatattggaaagagttgagatttgcccattataaattatgataattacccttcaaagaagatttcctgtttcactttttggtctgaatttgtacatttgcaagtcaatttgcagccatttgtgcctgggttgtttttcttggctgttttctctgagagcggcctgcacgagaagaagggaatgaaacccaatattgaagggaatctgcaagattcagctccactcagctcagtgaccagatgaagagctgttaggaatttgaagaaagttgttacttcaagccatttggtccttcttaaaactatcaaatgttaaatatctaaacagaatgaaattcaaatgagtagaaataaatgaaatatttctatagtgaaatattccagataataccttccctatcatccatttttataaagtttcctaaagcaaatccttgaaagattctaattttaagttgaacatgtacatgtaatcattaacatttctcttagagttggactttgcttttgctgcaggctttgaaataaccataaagtagaactttttattttatttatagaacattctaggtattagtttggtagaaaaccatctacgtgggatgattttctaaaaacccactcatcacccaaaaatacatccctatcagcctgaaattcttcaataatagattctcaccacatctagttgggcaaatgccactgaaatagtacattagaacccccacccgtcaattgcttactttccaacatggtaatgactgtgaggtcgactcagagcaaatttgagcaaagaaagtaagaaatgggctgccacgtgatataacatctgagcagaaggaactagtatgtggataaagacattaatactatactgggttttaaaatcagctcttcaggtttaagatgcttattaatccatctgtctaatcactgacttgttcattctttcatctacatagtacattttctacaaggtcttgtactaggttccttaggggatgaaaagatgagtaagtcgtagtcctggccctcttgtgggggaaacagacaacaaaataagcaatggctcagcaatcttccaagtgttaagagagatgcaactcgctgtggagtacagggggacaagagtggataattatggctgtgggagctaagggaggattccaaaataagttagcatttgagattggtgaagatagctgaggaacggagcattccaggcagagtgaaaaccacattgcccaggacctgagttaaagagcatggtgcacacagggagactgagctggccagggatgcctgaaacttcaaaagtctaaagtatcttgaggaaagtatagaaaatgagtctgaataagcaagtatacagataatagtctttaggccacctaaaggagagggatttgtgcatgcctgtgacacctttctcttcattcttctgtgcctctatgattgttacaccttttttattttttttcttttttgagacagaattgctctctgttgcccaggctggagtgcagtggtacgatctcggctcactgcaacctctgcctcccaggttcaagcgattctcccgcctcagcctcctgagtagctgggattacaggcacatcccaccacgcctggctaatattccttttttcgtttttgagacaagattttgtcatgttgcccaggctggtctcaaacccctaggctcaagcaattcacttacctcagcctcccaaaatgctgggattacaggcatgagctaccgcatccggcctccatttattatttaagtattggtgtttttctgggtctggatctcgccttgaccctcattagtcttcctttccttcggcaaacccttgtggtgtcaaagatgatgatgacttccagatccctggcaccagcacacacctctcttctgtgctccatgtttcaaatgcctgctgatcatctctcccacatgtctcacatggacctcaaacttaacttacgcctaaggtaaacttgttaaagcctattttaaaacaagtcttaatcttcattaaggcttctccacacacttattcacccaaattcgaaatctgagagctgttttcaacctccccctgttcctgttcctgacttcaacattccattgattagtaagttctgtcaattctacctttaaacatcaaagtctgttctcatcatccctactgacactgccttagtttgagtttctccatttgtaatttaagccagaaattctcaaactttagcttgcacaaacatcacctggagagcttgttaagacaccagagattctgagtctgtgcttctggggtggggcctgagagtgcatttccaacaagaccccaggtaatgctgatgtttgggtccatggaccacaggtttcgcacttcaatttattctaattgcttcttacctgtcctcctgctttggcttcacaagctccaatgtggttaatgaagttatcgttccaaaacaatttttactgtgtggcttttctgcttatgtattttataggactctgcattgcctgcaacacaaagcccagatacctttcctggcatgcaaggcctttggcagtctgactccagcttagttttccaatctcatctcttgccacttctccctgtaccttaagtaatttaaattatggccaggagtggtgcctcatgcctgtaatcccagtactttgtgagaccgaggcaggcagatcacgaggtcaggagttcgagaccagcctgaccaacatgacgaaaccccgtctctactaaaaatacaaaacttagccaggcatggtggcatgtgcctgtaatcccagctactcaggaagctgaggcaggagaattgcttgaacccgggaggtagaggttgcagtaagctgagattgcaccattgtactccagcctgggcaacagagcgagactccatctcaaaaaaaaaaaaaatatatatatatatatatatatagatagatagatagatagatagatagatagatctgtgcttccttgttcccatgttttgtacatgctatttctcgcatctgcaatgtccattctcttcttccattccccaccttttcctcactcccaaatggataactcacacttgctctttaagattcaacttacatattattttctttgtgctccagtagctctttgcatttctcttataggaattaccatgttgtgtcttgtctgtctgttagtttctctcataagaatgtaagttcatcaaaggaatggactgtgtctcattttagctttgtttctgagcccaaagctagaattgtgtttgtttagcacaatgcttggccacagcaagcatctatgatgttgtttaaactgactttaatcctataaatcaatgtctcccctcaaatgtgtcccactagtaccaggagatttttcaggaaaatgtcctatagtctaataatttggcaaatcacaaattctatgtaatagtgtatataggctctagaaagactagcaaaaaaaaaaaaaaaaaaacctgtcaagttttgtttaacacagtattaccaaaaagtatttgattccacaattccttattcctccaccatcccagaacacatattgacaatgttttctatggacatcaatttttaaagtgctcttgaagtcaatagggcaaggtattggtgtcttagaaagataatcttggctgcaaagtggaggataatctgagatttggggagaggatgggccaaggggctaggagagtagtaataccagggacagatatcaaaggcctgaattagtcagtgaagtagcagttggaagaggtcatatctgtgtgtgctcttatacaatataataagcagggacctgtgggaaattggagatggaaaagagaaagacattgaaaattaccatgaggtttacttagctcttattgcgttttgtattagggaagaatatatgtgtttccatgaagtattagacagtataggagtttgggaaatacatcgcacccaacaaaattttttggagtcaattggtcaataagctccaaatcccacattactctccattatcgagttcaagaaatggggaataaacccttttatcttcccctttcattttctggctcacataaaacaaagcccactagccctggttttagtcccttgataaatattcactatgtgcccaccacagtgcctaagaagtatcagtttatggcagtggttgttaaccgggcgattttgctccttgggacatttgacaacgtctggaggcatttttaattgtcacgctgggggagtgctactggcatctagtgggtagaggccagggatgctgttaaacatcctgtgatacataggacagccccctataacaaagaattatccatcccaaactgtcaatagtgtcagagttgcaaaaccctgatatgcaatgaaaactcaacacaattattctgacaattgaagagcagcgagggttttgcttaaattattagaataacagcctcatttctgggcttcgctttgttaatataggcctagccaggtggtgttgaataagcattagttcatgatttaatcttagttcctttctctcccagtgactaattataagtaatttgaattgctatgtgtgatcaacgatgtaaaagtaggacaaatgcacctctaatgagactatattagactctgaacttacagtttccccgaaagaggttgccctaagtatagggatacagaggaaggggcatgggttgctgagacaagtgtcctgagtttgagtcccagctctgctgcttactgactccatagccactataagtttactctgagcctcagtttcctcattgtgaaatgcagcaataatttcctcaccaagacaccgtgaggattgcacaaagtaatagcaaagtcatatactcaacaaatatttattctattcctattgtgtgtcaggccatattttcagtgctgggctacattcattgacaggacagacaaaaatatctacccttatggagttgggaatggttggagagaaagacaataaacaagataaataagtaaaatatagagagtgttaaatgacgttaagcgctatggaaaaagacaaaacaagaaagaggaatggtgcaagttggtacaattttaaatcagaatggcaaagaaggctcatttgagatgacatttgactttgaagaggagaggaagtgaggcatgccagtgtctgtggaaagagcattccaggcagaaggtcacagcatttgcaaacgtcctgaggccagatcatgcttggagagttcaaagaacgtcaagagagccagtgagcaaggggaagagaaaggaaggggagaagtgaggtgggagaatggagacagattgtgtgagaccataaaggccattgcaataaccctgaccacataaatgttagctagttattattattgcctataggtaaaatgttccagtgtgcattatgcctcaaataagagtgtttcgtaaaccctccttcaaaatcctgtgcccttctaagaatgtttttaaagatatccctcatttaaacaaaatggcaaggcgtctaagtgcaaaggacaaaattccagaattctcatggccaatctgctctgtgggttcttttcttgcaacagccacccactctgcctgggagccccactcatggagggggggcaggaaaaagtcaactcgggttttcttttttccccctcttcatttctacaataggaatagcttttccaagctgcccgagaatttaaagtacaccttcttttgttgtgctgctgatacagtcgcttttgctcttacaaattcccagagaaaatttatattcattctgaaaacaggaaagctttatttaaatacaacactatcacccaatgactgaatagatacagattaataattcctaagatttttagcagaaatattcaaaaaccttttcatgatatacaaaggcttttattttaatgtcaagtctcactttatttccttctctggggcataggaaaggagaagcaggacccagtatttttacttgcagtaaacacactttgccaataaaaccataaatgataataatttttttctaaaggtctctggcttactctgctctcatttgaacttgaaattcagaaggaaggaaagccttcccatttcagcaggagttgctgcaaacctgctaatcaaaataagtcatttttttaaattataagtgaaagtttaaaccagacagagaattttcacttgcaataaggattttcaaacccaaatgtatcttctgtttttaaaatatactttttccctattaaaacatggttcagttctcctccaagtataataaaaagaggccatggtacaatctgagccccctcttttattctgtgttttgtagctaaaggtcttcaataaaataccccagagctaaaaacaaagaactgtaaattagtttgagaattcttcatttaactgttagattcagcaagattatttagctgattctcccattccatgcatttcatgaattaaaaatagacctctgccactgacctattaccagcaccatgtctgtctacctctctctctctctctttctcacactgtcactcattctccctgtctaaagaatgactttctctggtaaagatgatatgatctatcctgatctgagggaagtgattcccagaaatctgtggtcataaggtcctaaaaacatggtatggctttagctggagagtcacactgtccccttatgatttttagggacgttcttgcatcagggcaagaataaacacagagagtcacttcctaatctagtgcacacatttttttttggtccatgtcccagacaactgcttagtccacgttcccaggagaagcacctctctcacagcatgtttgtacagaccaaacttgcttctcacgaggcagtgcaaccttatttggatgccctgttcatttcttcatatttaagaaaaggttcaaatttccatgtgacatgggcagagatctctttctcttaacaggatcccatttctgaatcacctcttgattccttggagaagaaaatctatgagagctaaacttactcctgactctgcaaattgcatcctgttttgtatttatttccttttccttaaatttgcaggaagaaatataaggtttttaaagacaccaatgacttttcctcctttgagctgctatgtcagaaatccttagaacatgtaccactacaacatacacaaactatggccatgtcttaaattatccaaaaagttcttatggccttcctttggtattctgttgtgaaaataaataagtaaatacaaagatacatgttctgcctcacagaacttttgcaaactgcgaggatgtatcttacatttaaattttggtaattttttttctgtgcatctctttcaagtaaaatcttgtttttgttccagcatggtcctagttggaaagatatagagaaaaaaatgagaattttttttttgcaaaagaaaattgaaattctgtctatctcttaaactgctcctaccatgttttaagagggtctgttctgatcagcacaccaagtatttctaatgaattagggaagaaattaccattatgtctggtcatttatctgaagcttgctgccgaggatgcaagatagacatgtaatccacccttagccatattctccagctcccttcagtccggattgtgagtagagttgctataaacatatggaggaaagcttatgaaagtaaatctgagtctgactcataacaatgtctctacagtcctatggaggatacgtttcatcactggcccttgcatctggaactggtcttttcaaatgtggtatagcagtggctccagtctccagctgggaatattacggtatggaaacattcattaatgttactgtgaaagcatttaacacgtaattactctatatctgatgttgctagaaaaatggattatgcagtaaaattcaccccagcatcactggggaaaatataatttataaacaaagttgtttatatcatttattataaacatgttgtaacaatcaatgaaatctcaagagtgaggaaaggctcatccataatccactatatcttattttctcccagaaaccttgagcccctgccgaaccaaatctttaaaatcagagctaactctgtttttgcaggcacacagctacagcataattggcttttaagcacatcttcagcacggtcaggccagaggccaagcagttcacctccacgccacagttagcaattgccagttccattgctccagaaatcaaagaaacaaaaactttgttggagaaatactcctgtttgtgcctatttttatcattacatcaatgcaggatgattatttggaaggaattctaataattatacaaaataactctaataattttacaaaaattagcttcatcagattctctcctctatattggggcagaaataaaacaattgtgacttgtaggggatcttgattaagaaaaaaagtgtgtgcatctatgtacgtgtgtagggggaatagatttgagggacatattttcatcccagatgaaataagacaccagtaaaagtcctaaagatggctggatttgtgattttctttaaaatatattgagaccattctacaggtcattcctacttcaacattcttccctaggagggtagaagtgaaaaatattaagaatagtcatagaaagaaaatgtttagatgctcctgtttccataatctcttccttcatacttctctagctctattgaggctaattgcagagatgacacatattctttgaggccaaccattatagcaaagtaaagcaagaatttacctcaagagctcaagatgggaaaattgagaaacagttattaaactagaaggtcttgttggttaatgaaatgacattcatcacatgcaaacaatgcatattctcatatattttatagcgtctgtctacacagagagattcatgggtctcccaacaaaggatgataatcttgagcactataaggtaagtgtttccattttatttaggttaataattaatttctaatatgctaaataacttaaaagcagtagttcagaatttacagttttaattctgggcccccaagcaaatatcatcttttgttaattgcaaaacacatcactagaaagggttttactccttttggattctgttctttcttttcccatcctcccttccctaacctctcttttcccaatcttcctccccacccccaacccctacacagcttgagcgggcatcatttatgcctaaggtctcactcatatgtaacatatgacacataaactttcagtttttcaaactaagaatcagatggaagtaatgcaaatgatgatgaaaatgtgttgctctgactcttaaatatttatcaagcaaagcaatacataatttcagtattggtgctaagtccattgcacaatgatttagctagttttctattagaacttgcagcaactaaaatgttaatttttttctcttttccagaattcaactgtgatggcaagagcagaatatttcagaaatgtagactatcttctcatccacggaacagcagatggtgaggtttaagcaagactcttacacataaaaatactgagccagcatcactttgtttaagaaacattaagcatcctcctatgcctatgctcagggtcagtacctaagagtcagggacatttccatatctgagtgtgattactcactgctgacaatgtcaaatgcatgactagataattgttggtgttcatttccaggcagtacattgtcactctatgctttctggtatttagcagcactttaactctctctctctttgttgcagataatgtgcactttcaaaactcagcacagattgctaaagctctggttaatgcacaagtggatttccaggcaatggtacataattttgttttactcatgttcatggccttagaccctcaagctaaaaggaggggttcatatttttattttttttaacttttaaagtaaaatctgacaaagaggctgcaggatagagtaggcagaacattagcaaatgaatcttggatctttctttaactactgaaggacccgaggcaaaccagatcacctctcgtgttcccagttgagtcatgtaaaataaaggagttcacaagattaaatgatcttctagttcaaacatccagtagttctgaaatcctatgtgaataagtgcaattatatgcttagagtccctgcaaaacctgagcatccttaaacagctagaagattctgtttgttcagtaagaagcagttgcagaatgtcatcacaaacctccaaggctccctgaaatcatatggctccatttgtggttagagaacttttcacctatgtattctcatcccctggcatacacctggcttatcacagatgctcagcttcttactgagtgaatgaatgaattagctaaatgtaaaatccaggttgcttgaggaatgagaaattgaaccagaaaagaaaataaagtgaaagccaaagttgggggcggggcagcattcttggtagctaccttttcctctcacaaatgctccctcaattgcttaagttgaacaagaaccccgaaaaggagacccccatcttttcaacatttgtttttaaaaaatgtactgactgggaaagaaatgggggagggggtataagaagaagttggttaaggggtacaaaagacagaaaaaaataagttctagtatttgaaagtacagtagggaaattatggtcaatgataattgattgtataattcaaaatagctggaagagaataattataattttcccaacataaaaggaaatttgtttgaggtgatgggtatttcagttatcttgatttgatcattacacattgtattcagctatcaaaatatcactgtacccccaaaaagatacacaactattatatatcaaatttttaaaaatatacaaagagggaggtgagctttttctgaccttttttattgtgatccccaaagtaaattcaacttagtaaatatatattattacatcgtgaccactgatacctttattcagtgaaagcaataatcttagtggtatctctccaagagtgaagtttaaagttaaaatactatctttcactttttaaaaatgctatttttaactcctataaagggtgtttatggctatatatgaatacattatattgtggattttctgcaacagagaggaattagattcacaggctgaaagcctcatggaatttttgagctacaaggaatcttagggaaaatcaactccaacccctttgtttttcaaatgaagaaaccagactccaaagaagcaaaattaacagacccagtgcatcacttctggcaactctgatgcctgatcctgatcttccaggatgctggatgatgcgagatctcagccactgagatctaaaatatgtttaaagcatgtgaaacagtagtggaggttaacgaaagctcttcctgctgtaaatatttacatgtaagctattttacttatttgagtgttcttaggtacagtttccccataatagaaagattatcaggtcctgagagcacaaaaaagtccaagctaaatgcatttgatgactgcaggaataaaacaagcttttttctttgtgtttcagtggtactctgaccagaaccacggcttatccggcctgtccacgaaccacttatacacccacatgacccacttcctaaagcagtgtttctctttgtcagactaaaaacgatgcagatgcaagcctgtatcagaatctgaaaaccttatataaacccctcagacagtttgcttattttattttttatgttgtaaaatgctagtataaacaaacaaattaatgttgttctaaaggctgttaaaaaaaagatgaggactcagaagttcaagctaaatattgtttacattttctggtactctgtgaaagaagagaaaagggagtcatgcattttgctttggacacagtgttttatcacctgttcatttgaagaaaaataataaagtcagaagttcaagtgcta

SEQ ID NO: 2 - DNA, 22 bp, Artificial Sequence
  Description of Artificial Sequence Synthetic primer
atgagcttcctcgtccaattca

SEQ ID NO: 3 - DNA, 23 bp, Artificial Sequence
  Description of Artificial Sequence Synthetic primer
agaccaccagagagcatattttg

SEQ ID NO: 4 - DNA, 23 bp, Artificial Sequence
  Description of Artificial Sequence Synthetic primer
gattagcgatgatgaaccaggtt

SEQ ID NO: 5 - DNA, 21 bp, Artificial Sequence
  Description of Artificial Sequence Synthetic primer
cctcccatctccttcatgaca

(19) United States
(12) Patent Application Publication
(10) Pub. No.: US 2024/0115670 A1
(43) Pub. Date: Apr. 11, 2024

(54) FIBROBLAST ACTIVATION PROTEIN MODULATION TO ALTER IMMUNE CELL MIGRATION AND TUMOR INFILTRATION

(71) Applicant: Georgetown University, Washington, DC (US)

(72) Inventors: Louis M. Weiner, Washington, DC (US); Allison Fitzgerald, Washington, DC (US)

(21) Appl. No.: 18/263,129

(22) PCT Filed: Jan. 27, 2022
(86) PCT No.: PCT/US2022/014077

§ 371 Date: Jul. 26, 2023

Related U.S. Application Data
(60) Provisional application No. 63/142,300, filed on Jan. 27, 2021.
(60) Provisional application No. 63/239,526, filed on Sep. 1, 2021.

(51) Int. Cl. A61K 38/48; A61K 38/19; A61K 39/00; A61P 35/00

(52) U.S. Cl. CPC A61K 38/4813; A61K 38/195; A61K 39/4611; A61K 39/4613; A61K 39/4631; A61P 35/00

(57) ABSTRACT

Systems and methods are disclosed for the treatment of cancer. Specifically, techniques are disclosed for treating cancer through the administration of genetically modified immune cells that overexpress fibroblast activation protein. In other embodiments, the techniques include the treatment of cancer through the administration of fibroblast activation protein inhibitors to the tumor site.

### CROSS-REFERENCE TO RELATED APPLICATIONS

This application claims the benefit of U.S. Provisional App. Nos. 63/142,300 and 63/239,526, filed on Jan. 27, 2021 and Sep. 1, 2021, respectively, the contents of which are incorporated herein by reference.

### GOVERNMENT LICENSE RIGHTS

This invention was made with government support under Grant Nos. R01 CA50633 (LMW) and F30 CA239441 (AAF) awarded by the National Institute of Health (NIH) and the National Cancer Institute (NCI). The government has certain rights in the invention.

### FIELD OF THE DISCLOSURE

The field of the disclosure relates to the treatment of diseases such as cancer. More specifically, the techniques disclosed involve treating cancer through the administration of modified immune cells that overexpress fibroblast activation protein (“FAP”) or the administration of FAP inhibitors to the tumor site.

### BACKGROUND OF THE DISCLOSURE

FAP is a 97-kDa type II transmembrane serine protease. FAP is a member of the propyl peptidase family, which also contains dipeptidyl peptidase IV (DPPIV, CD26), DPP7 (DPP II, quiescent cell proline dipeptidase), DPP8, DPP9, and prolyl carboxypeptidase (PCP, angiotensinase C). Within this family FAP is most like DPPIV, sharing 70% amino acid sequence homology (Leslie A Goldstein et al., 1997). These proteins contain a catalytic triad of serine, aspartic acid and histidine. (Rosenblum & Kozarich, 2003) The serine acts as a nucleophile, cleaving N-terminal Pro-X peptide bonds, where X is any amino acid except proline or hydroxyproline. FAP contains dipeptidyl peptidase enzymatic activity and endopeptidase activity, sometimes referred to as gelatinase activity. Both FAP and PDDIV have dipeptidyl peptidase activity, but endopeptidase activity is specific to FAP. Hence, endopeptidase activity is the basis for FAP specific detection methods and FAP specific inhibitory molecules. FAP's endopeptidase activity prefers amino acid sequences of Gly-Pro-X, is most effective where X is Phe or Met, and least effective when X is His or Glu (Collins et al., 2004). Furthermore, FAP is ineffective with large charged amino acids at position P4 and P2′ (Aggarwal et al., 2008; Edosada et al., 2006; C.-H. Huang et al., 2011).

While FAP's substrate repertoire is largely unknown, some substrates were identified by a study that screened known DPPIV substrates for cleavage by FAP. This study demonstrated FAP's dipeptidyl peptidase activity enables it to cleave neuropeptide Y, peptide YY, substance P and brain natriuretic peptide 32 (Keane et al., 2011). Known substrates of FAP's endopeptidase activity include denatured collagen type I and III (the components of gelatin) (Christiansen et al., 2007; M. T. Levy et al., 1999), α-2 antiplasmin cleaving enzyme, and recently discovered fibroblast growth factor 21 (Dunshee et al., 2016). Of note, FAPs ability to cleave collagen is dependent on prior collagen degradation by matrix metalloproteases or heat.

FAP's ability to cleave α-2 anti-plasmin has been extensively detailed. During tissue repair, fibrin is deposited to form a fibrin clot. Fibrinolysis is the natural process in which a fibrin clot is dissolved by plasmin leading to scar resolution. A-2 anti-plasmin is an inhibitor of plasmin and therefore reduces the rate of lysis of the fibrin clot. Cleavage of a2-antiplasmin by FAP converts a2-antiplasmin into a more potent inhibitor of plasmin (K. N. Lee et al., 2004). Therefore, soluble FAR, referred to as APCE, functions to enhance clotting.

Research with a catalytically mutant FAP (in which the Ser 642 is mutated to Ala) has suggested that FAP can have functional impacts independent from its enzymatic activity. Mouse melanoma lines transfected to express FAP had reduced tumorigenicity. This effect was enhanced when the same cells were transfected with catalytically inactive FAP. While this study contradicts many reports of FAP being oncogenic, it suggests that catalytically inactive FAP can still induce biological effects (Ramirez-Montagut et al., 2004). In a similar study, breast cancer lines transfected either FAP or catalytically inactive FAP grew more rapidly in vivo, were more invasive on collagen gels, and had greater degradation of extracellular matrix in comparison to nontransfected cell lines (Y. Huang et al., 2011), suggesting enzymatic activity was unnecessary for the observed phenotype. Another study demonstrated that breast cancer cell lines transfected with FAP and catalytically mutant FAP both had increased cellular growth and motility and both proteins activated signaling molecules PI3K and MMP2/9 (B. Lv et al., 2016).

### SUMMARY OF THE DISCLOSURE

Provided herein are techniques for treating diseases. Specifically, techniques are disclosed for treating cancer through the administration of genetically modified immune cells that overexpress fibroblast activation protein. In embodiments, the techniques are intended to treat cancer, including pancreatic cancer.

In certain embodiments, a pharmaceutical composition is disclosed comprising genetically modified immune cells, where the modified immune cells overexpress fibroblast activation protein (FAP).

In certain other embodiments, a method of disease treatment is disclosed, in which the steps comprise administering a pharmaceutical composition comprised of a therapeutically effective amount of genetically modified immune cells, wherein the genetically modified immune cells are altered to overexpress fibroblast activation protein (FAP).

In yet other embodiments, a method of preparing one or more genetically modified immune cells is disclosed, in which the steps comprise transfecting a vector containing a gene for fibroblast activation protein (FAP) into one or more immune cells in a media, replicating the one or more immune cells transfected by the vector, and isolating the one more immune cells transfected by the vector that overexpresses fibroblast activation protein (FAP).

In certain embodiments, the immune cells are comprised of genetically natural killer (NK) cells, T-cells, or a combination thereof.

In certain embodiments, the immune cells are comprised of CD4 T-cells, CD8 T-cells, or a combination thereof.

In other embodiments, natural killer cells are selected from NK92, NK92-GFP, NKL, YT, KHYG-1, NK92-CD16V, or a combination thereof.

In other embodiments, the genetically modified natural killer cells are derived from normal human donors.

In yet other embodiments, prior to genetic modification, the natural killer cells are isolated from peripheral blood, pluripotent stem cells, or a combination thereof.

In other embodiments, the genetically modified natural killer cells are further modified to express a chimeric antigen receptor (CAR).

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a schematic diagram of FAP domain structure (top) and ribbon models (bottom) depicting the FAP dimer. The seven-bladed β-propeller domain, a/s hydroxylase domain and β-propeller blade are highlighted;

FIG. 2 shows a schematic diagram of potential signaling pathways affected by FAP that are responsible for the tumor promoting phenotypes associated with FAP expression;

FIG. 3 shows (A) Schematic diagram of in vitro coculture system. (B) Representative photomicrographs of hematoxylin stained PSCs cultured alone (left) and cultured with NK92 cells for three days (right). Bar, 200 μm. (C) Graph of average number of PSCs and NK92 cells remaining following 3 days of coculture as determined by flow cytometry. Data compiled from multiple experiments of PSC alone (n=1), PSC+NK92 (n=3). (D) Representative phase contrast photomicrographs of PSCs cocultured with NK92 cells with (right) and without (left) fluorescent imaging. Bar, 200 μm. (E) Graph of percentage of apoptotic PSCs (early and dead) in PSCs cultured alone or with NK92 cells at E:T ratios of 1:1 and 4:1 for four hours. Data are compiled from four independent experiments (n=4) and shown as mean+/−SD. ***=p<0.001 initially by one-way ANOVA and subsequently by Tukey's multiple comparison test. (F) Representative flow cytometry profiles of Annexin V versus Sytox staining showing percentage of live, necrotic, early apoptotic and dead apoptotic PSCs when cultured alone or with NK92 cells at an E:T ratio of 1:1 and 4:1 for four hours. PSCs were selected using a DiI positive, GFP negative gate. (G) Representative photomicrographs of hematoxylin stained PSCs cultured alone (left), with primary NK cells (middle) and with primary CD3+ T cells (right) for 24 hours. Bar, 500 μm. (H) Annexin V flow cytometry assay showing percentage of live, necrotic, early apoptotic and dead apoptotic PSCs when cultured alone or with primary donor NK cells at an E:T ratio of 4:1 and 10:1 for four hours. PSCs were selected using a DiI positive, DiO negative gate;

FIG. 4 (A) NK92 cell line expression of NKG2D by flow cytometry. (B) PSC expression of MICA/B by flow cytometry. (C) Effect of 1-10 ug of anti-NKG2D blocking antibody on NK92 lysis of PSCs as determined by annexin V flow cytometry assay;

FIG. 5 (A) FAP activity assay showing FAP activity upon co-culture of PSCs with NK92 cells for one day. Data are compiled from two independent experiments, each with three technical replicates (n=6). Data are represented as mean+/−SD. ***=p<0.001 by unpaired two-tailed t-test. (B) Quantitative real-time PCR analysis of FAP expression in PSCs cultured alone or with NK92 cells for three days (n=3). Data represented as mean+/−SD ***=p<0.001 by unpaired two-tailed t-test. (C) Representative western blot demonstrating decreased FAP expression in PSCs following coculture with NK92 cells, GAPDH was used as a loading control. Experiment was repeated twice. (D) Quantification of FAP levels seen in western blot in C. FAP band intensity normalized to GAPDH band intensity. (E) Representative photomicrograph of immunohistochemistry for FAP in PSCs grown in vitro;

FIG. 6 (A) Three different anti-FAP antibodies were assessed for their ability to detect FAP by western blot. Known FAP-expressing primary culture pancreatic stellate cells (PSC; ScienCell, Carlsbad, CA) were used in triplicate to test ab207178 (abcam, Cambridge, MA), MBS303414 (MyBiosource, Inc. San Diego, CA) and ab53066 (abcam, Cambridge, MA). One known FAP-negative cell line (PANC-1) was used as a negative control, and 27 ug of recombinant FAP (R&D Systems, Minneapolis, MN Cat #3715-SE-010) was used as the positive control. All three antibodies detected rFAP and bound to PSC preparations at the same molecular weight as recombinant FAP. None of the antibodies bound to PANC-1. (B) Western blot demonstrating rFAP binding specificity of ab207178, with no binding to rDPPIV. 27 ug of each designated recombinant protein was loaded into the gel. (C) Real-time quantitative PCR analysis of FAP expression in three PSC preparations and PANC-1 cells demonstrating PANC-1 cells lack FAP at both the mRNA and protein level. Data represents mean+/−SD. Each point represents an independent experiment and is the average of three technical replicates;

FIG. 7 (A) Quantitative real-time PCR analysis of FAP expression in NK92 cells pre-PSC exposure and post-PSC exposure (n=3). Data represented as mean+/−SD. ***=p<0.001. Data analyzed with two-tailed unpaired t-test. (B) Representative western blot of NK92 cells cultured alone or with PSCs in a transwell coculture system. Experiment was repeated twice. (C) Quantification of FAP western blot seen in B. FAP band intensity normalized to GAPDH band intensity. (D) Western blot of FAP protein levels in response to increasing concentrations of IL-2 4 hours after exposure. (E) Western blot of FAP protein levels in response to increasing concentrations of IL-2 24 hours after exposure. (F) Representative western blot demonstrating FAP expression in four human NK cell lines: NK92, NKL, YT and KHYG-1 (n=3 replicates). (G) Western blot analysis of FAP expression in three T-cell, B-cell and monocyte cell lines. (H) Quantification of FAP levels seen in western blot in F. FAP band intensity normalized to GAPDH band intensity. (I) Broad cell line encyclopedia RNAseq data showing FAP mRNA levels in the same T-cell, B-cell and monocyte lines. Cell lines lacking data in database are designated as N/A. (J) Table containing annotation information for murine immune cells tested for FAP expression. (K) Western blot analysis of FAP expression in various murine immune cell lines. (L) Quantification of FAP levels seen in western blot in J. FAP band intensity normalized to GAPDH band intensity;

FIG. 8 (A) Western blot demonstrating FAP expression in three separate healthy human donors. (B) Western blot demonstrating FAP expression in two additional healthy human donors using two anti-FAP antibodies. (C) Flow cytometry analysis assessing purity of primary donor immune cells. (D) Western demonstrating FAP expression in NK cells, but not other immune cells, isolated from PBMCs from healthy human donors. Included in the blot is a positive control (NK92) and negative control (PANC-1) cell line. Blot representative of two different healthy donors;

FIG. 9 Single-cell RNA-seq analysis of FAP expression in different cell populations present in (A) primary tumor and (B) lymph node metastasis of head and neck squamous cell carcinoma patients;

FIG. 10 (A) Western blot demonstrating FAP is detected in total cell lysate (T) but not in nonbiotinylated intracellular protein compartment (IC) in four human NK cell lines (NK92, NKL, YT, KHYG-1). (B) Flow cytometry analysis for FAP expression on the surface of PSCs (positive control) and four human NK cell lines;

FIG. 11 (A) FAP mRNA expression (RSEM units) in pancreatic tumor specimens (gray box) ranks highest among all solid tumors (TCGA). (B) Pancreatic (PAAD) and stomach (STAD) adenocarcinoma are the only two solid tumor types that have significantly increased (p<0.01, red asterisk) FAP, DPP4 and DPP9 mRNA expression in tumors compared to healthy tissue (TCGA). (C) Quantification of FAP, DPP4, DPP8 and DPP9 expression in pancreatic tumor (pink, n=179) compared to normal pancreas (gray, n=171) (TCGA). (*p<0.01);

FIG. 12 (A) Level of FAP expression in NK cell lymphomas (n=22) and NK cell lines (n=11) as determined by Affymetrix gene expression array. (B) Heatmap of gene expression array data. Data are shown as z-score scaled values. (C) Top 19 genes that are significantly correlated with FAP expression. (D) Top GO pathways that significantly correlate with FAP expression;

FIG. 13 (A) Fluorescent peptide dipeptidyl peptidase activity assay demonstrating FAP inhibitor (Cpd60) inhibits FAP but not DPPIV. (B) Schematic of live imaging of primary human NK cell migration on stromal cells. (C) Representative phase-contrast images from live imaging showing multiple colored tracks. Each color track represents the migration path of a single NK cell. (D) Rose plots with overlaid NK cell migration tracks. Each treatment group contains 30 different NK cells from a single healthy donor. The average velocity (E), accumulated distance traveled (F) and Euclidian distance traveled (G) by primary NK cells treated with either Vehicle or 10 uM Cpd60. Each point represents a single NK cell. Each condition contains 90 NK cells with 30 NK cells from three separate donors;

FIG. 14 (A) Schematic representation (top) of zebrafish injections. Fluorescent and brightfield overlay image of Tg(kdrl:mCherry-CAAX)y171 zebrafish embryos expressing endothelial membrane targeted mCherry (bottom). (B) Representative images of caudal hematopoietic tissue immediately after NK92-GFP injection into the pericardium. (C) Still image taken from confocal time-lapse video demonstrating NK92-GFP extravasation from mCherry labeled vasculature. (D) Representative fluorescent microcopy images demonstrating NK92-GFP extravasation. Extravascular image was taken approximately 5 minutes after the intravascular image. Images were taken at 20×. (E) Representative fluorescent microscopy images of NK92-GFP injected zebrafish in 10 uM FAP inhibitor (Cpd60) or vehicle showing NK92-GFP cell intravascular or extravascular localization 1 hour after injection. Images were taken at 10×. (F) Quantification of extravascular NK92-GFP cells in zebrafish injected with NK92-GFP cells 1 hour prior to imaging. *p<0.05 analyzed by unpaired two-tailed t-test. Data are aggregated from two independent experiments, each with 10 fish per treatment condition and quantification was done blinded to treatment conditions;

FIG. 15 (A) Schematic representation of experimental methods and analysis. (B) Average continuous GFP intensity measured along PANC-1, PSC or PANC-1+PSC spheroid equator. (C) Average GFP intensity in the edge, mid-edge, mid-center and center regions of PANC-1, PSC and PANC-1+PSC spheroids. PANC-1 n=6; PSC n=6; PANC-1+PSC n=12. *p<0.05 as determined by ordinary one-way ANOVA followed by Tukey's multiple comparison test. (D) Representative fluorescent images of NK92-GFP cells infiltrating into tumor spheroids cultured in vehicle or 10 uM FAP inhibitor (Cpd60). (E) Average continuous GFP intensity measured along PANC-1, PSC or PANC-1+PSC spheroid equator cultured in vehicle or 10 uM Cpd60. (F) Average GFP intensity in the edge, mid-edge, mid-center and center regions of PANC-1, PSC or PANC-1+PSC spheroids cultured in vehicle or 10 uM Cpd60. PANC-1+vehicle n=6; PANC-1+Cpd60 n=5; PSC+vehicle n=6; PSC+Cpd60 n=6; PANC-1+PSC+vehicle n=12, PANC-1+PSC+Cpd60 n=12. *p<0.05 as determined by unpaired two-tailed t-test;

FIG. 16 (A) Schematic representation of experimental design. (B) Still image from confocal time-lapse video of NK92-GFP cocultured with PANC-1 or PSC clusters embedded in 3D matrix and vehicle or 10 uM FAP inhibitor (Cpd60). (C) Representative immunofluorescence images and quantification of NK92-GFP cell infiltration into PANC-1 or PSC clusters after 24-hour coculture with vehicle or 10 uM Cpd60. PANC-1+vehicle n=29; PANC-1+Cpd60 n=45; PSC+vehicle n=11; PSC+Cpd60 n=14. PANC-1 data aggregated from two independent experiments. (D) Representative immunofluorescence images of phalloidin and cleaved caspase 3 staining in PANC-1 cell clusters cocultured with NK92 and vehicle or 10 uM Cpd60. (E) Quantification of cleaved caspase 3 intensity staining in PANC-1 cell clusters cocultured with NK92 cells or donor NK cells. PANC-1+NK92+vehicle n=18; PANC-1+NK92+Cpd60 n=9; PANC-1+Donor NK+vehicle n=25, PANC-1+Donor NK+Cpd60 n=12. Donor NK cell data is aggregated data from two independent experiments that used different donors. *p<0.05, **p<0.01, ***p<0.001 as determined by unpaired two-tailed t-test;

FIG. 17 shows an exemplary pathway for FAP-mediated proteolytic migration of NK cells;

FIG. 18 shows charts demonstrating that human NK cells express catalytically active fibroblast activation protein. (A) Fluorescent peptide substrate assay demonstrating 4-hour coculture of primary pancreatic stellate cells (PSC) with NK92 cells increases dipeptidyl peptidase activity. Results are from two independent experiments. (B) qRT-PCR analysis of FAP expression in PSCs and NK92 cells before and after coculture. Results are from three independent experiments. (C) Western blot showing that four distinct human NK cell lines express FAP. (D) Western blot showing primary NK cells isolated from PBMCs from three different healthy human donors express FAP. (E) Western blot showing heterogenous FAP expression in multiple human immune cell lines. (F) Western blot showing FAP is only expressed in human NK cells and not in human T (CD3+), B (CD19+) or monocyte (CD14+) cells. NK92 cell line included as a positive control and PANC-1 cell line included as a negative control. Representative of results with two different donors. (G) Flow cytometry analysis assessing surface expression of FAP in human NK cell lines. Pancreatic stellate cells (PSC) included as a positive control. (H) Western blot of total protein (T) and intracellular (IC) protein isolated from human NK cell lines using cell surface protein biotinylation for exclusion of surface proteins. P value was calculated using unpaired two-tailed t-test. ***P<0.001, ****P<0.0001;

FIG. 19 shows charts that demonstrate In NK cells, FAP gene expression correlates with extracellular matrix and migration-regulating genes. (A) Level of FAP expression in NK cell lymphomas (n=22) and NK cell lines (n=11) as determined by Affymetrix gene expression array. (B) Heatmap of gene expression array data. Data are shown as z-score scaled values. (C) Top 19 genes that are significantly correlated with FAP expression. (D) Top DO pathways that significantly correlate with FAP expression;

FIG. 20 shows charts that demonstrate FAP inhibition reduces primary human NK cell migration. (A) Fluorescent peptide dipeptidyl peptidase activity assay demonstrating FAP inhibitor (Cpd60) inhibits FAP but not DPPIV. (B) CellTiterBlue cell viability assay demonstrating FAP inhibitor (Cdp60) has no effect on NK cell line viability. (C) Schematic of live imaging of primary human NK cell migration on stromal cells. (D) Representative phase-contrast images from live imaging showing multiple colored tracks. Each color track represents the migration path of a single NK cell. (E) Rose plots with overlaid NK cell migration tracks. Each treatment group contains 30 different NK cells from a single healthy donor. (F) The average velocity, (G) accumulated distance traveled and (H) Euclidian distance traveled by primary NK cells treated with either vehicle or 10 uM Cpd60. Each point represents a single NK cell. Each condition contains 90 NK cells with 30 NK cells from three separate donors. Data was determined to be non-parametric by Shapiro-Wilk test and a difference in means was determined by the Mann-Whitney test;

FIG. 21 shows images and charts that demonstrate that FAP inhibition reduces NK cell extravasation from zebrafish blood vessels. (A) Schematic representation (top) of zebrafish injections. Fluorescent and brightfield overlay image of Tg(kdrl:mCherry-CAAX)y171 zebrafish embryos expressing endothelial membrane targeted mCherry (bottom). (B) Representative images of caudal hematopoietic tissue immediately after NK92-GFP injection into the pericardium. (C) Still image taken from confocal time-lapse video demonstrating NK92-GFP extravasation from mCherry labeled vasculature. (D) Representative fluorescent microcopy images demonstrating NK92-GFP extravasation. Extravascular image was taken approximately 5 minutes after the intravascular image. Images were taken at 20×. (E) Representative fluorescent microscopy images of NK92-GFP injected zebrafish in 10 uM FAP inhibitor (Cpd60) or vehicle showing NK92-GFP cell intravascular or extravascular localization 1 hour after injection. Images were taken at 10×. (F) Quantification of extravascular NK92-GFP cells in zebrafish injected with NK92-GFP cells 1 hour prior to imaging. *p<0.05 analyzed by unpaired two-tailed t-test. Data are aggregated from two independent experiments, with a total of 19 fish per treatment condition and quantification was done blinded to treatment conditions;

FIG. 22 shows charts that demonstrate FAP inhibition reduces NK cell infiltration into matrix containing spheroids. (A) Schematic representation of experimental methods and analysis. (B) Average continuous GFP intensity measured along PANC-1, PSC or PANC-1+PSC spheroid equator. (C) Average GFP intensity in the edge, mid-edge, mid-center and center regions of PANC-1, PSC and PANC-1+PSC spheroids. PANC-1 n=6; PSC n=6; PANC-1+PSC n=12. *p<0.05 as determined by ordinary one-way ANOVA followed by Tukey's multiple comparison test. (D) Representative fluorescent images of NK92-GFP cells infiltrating into tumor spheroids cultured in vehicle or 10 uM FAP inhibitor (Cpd60). (E) Average continuous GFP intensity measured along PANC-1, PSC or PANC-1+PSC spheroid equator cultured in vehicle or 10 uM Cpd60. (F) Average GFP intensity in the edge, mid-edge, mid-center and center regions of PANC-1, PSC or PANC-1+PSC spheroids cultured in vehicle or 10 uM Cpd60. PANC-1+vehicle n=6; PANC-1+Cpd60 n=5; PSC+vehicle n=6; PSC+Cpd60 n=6; PANC-1+PSC+vehicle n=12, PANC-1+PSC+Cpd60 n=12. *p<0.05 as determined by unpaired two-tailed t-test;

FIG. 23 shows charts that demonstrate that FAP inhibition reduces NK cell infiltration and lysis of PANC-1 cell clusters embedded in 3D cell matrix. (A) Schematic representation of experimental design. (B) Still images from confocal time-lapse video 24 hours after coculture of NK92-GFP with PANC-1 or PSC clusters embedded in 3D matrix and vehicle or 10 uM FAP inhibitor (Cpd60). (C) Representative immunofluorescence images and quantification of NK92-GFP cell infiltration into PANC-1 or PSC clusters after 24-hour coculture with vehicle or 10 uM Cpd60. PANC-1+vehicle n=29; PANC-1+Cpd60 n=45; PSC+vehicle n=11; PSC+Cpd60 n=14. PANC-1 data aggregated from two independent experiments. (D) Representative immunofluorescence images of phalloidin and cleaved caspase 3 staining in PANC-1 cell clusters cocultured with NK92 and vehicle or 10 uM Cpd60. (E) Quantification of cleaved caspase 3 intensity staining in PANC-1 cell clusters cocultured with NK92 cells or donor NK cells. PANC-1+NK92+vehicle n=18; PANC-1+NK92+Cpd60 n=9; PANC-1+Donor NK+vehicle n=25, PANC-1+Donor NK+Cpd60 n=12. Donor NK cell data is aggregated data from two independent experiments that used different donors. Outlier's identified by Rout's method were excluded. *p<0.05, **p<0.01, ***p<0.001 as determined by unpaired two-tailed t-test; and

FIG. 24 shows a diagram of various NK cell types where increasing FAP expression can be used to enhance pancreatic ductal adenocarcinomas (PDAC) infiltration by activated NK cells.

### DETAILED DESCRIPTION OF THE PREFERRED EMBODIMENTS

In describing a preferred embodiment of the disclosure illustrated in the drawings, specific terminology will be resorted to for the sake of clarity. However, the disclosure is not intended to be limited to the specific terms so selected, and it is to be understood that each specific term includes all technical equivalents that operate in a similar manner to accomplish a similar purpose. Several preferred embodiments of the disclosure are described for illustrative purposes, it being understood that the disclosure may be embodied in other forms not specifically shown in the drawings.

### Definitions

As used herein, the term “nucleic acid” refers to polynucleotides such as deoxyribonucleic acid (DNA), and, where appropriate, ribonucleic acid (RNA).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

“Transformation” to any process by which exogenous DNA enters a host cell. Transformation may occur under natural or artificial conditions using various methods well known in the art for the insertion of foreign nucleic acid sequences into a prokaryotic or eukaryotic host cell, for example. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

As used herein, the term “expression” refers to any number of steps comprising the process by which polynucleic acids are transcribed into RNA, and (optionally) translated into peptides, polypeptides, or proteins. If the polynucleic acid is derived from genomic DNA, expression may, if an appropriate eukaryotic host cell or organism is selected, include splicing of the RNA.

As used herein, the term “overexpression” used with respect to proteins such as fibroblast activation protein, refers to the synthesis of excess protein in a eukaryotic cell. Overexpression refers to protein synthesis that is at least approximately X %, more preferably Y %, and even more preferably Z % in excess of natural production in the cell.

The term “transfecting” refers to a methods for introducing bio-active materials, such as nucleic acids, proteins, enzymes, or small molecules, into a cell. The nucleic acids may be DNA, delivered as plasmid or oligomer, and/or RNA or combinations thereof.

As used herein, “cell surface receptor” refers to molecules that occur on the surface of cells, interact with the extracellular environment, and transmit or transduce the information regarding the environment intracellularly in a manner that may modulate intracellular second messenger activities or transcription of specific promoters, resulting in transcription of specific genes.

“An effective amount” or a “therapeutically effective amount” refers to the amount of a therapy that is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

“At least one” means one or more (e.g., 1-3, 1-2, or 1).

“Composition” includes a product comprising the specified components in the specified amounts, as well as any product that results, directly or indirectly, from combination of the specified components in the specified amounts.

“In combination with” as used to describe the administration of the components of the present invention (1) with other medicaments in the methods of treatment of this invention, and means that the components and the other medicaments are administered sequentially or concurrently in separate dosage forms, or are administered concurrently in the same dosage form.

“Mammal” means a human and other mammals, or means a human being.

“Patient” and “Subject” includes both human and other mammals, preferably human.

“Chemokine” means a cytokine involved in chemotaxis.

The term “inhibitor” refer to a modulator that, when contacted with a molecule of interest, causes a decrease in the magnitude of a certain activity or function of the molecule compared to the magnitude of the activity or function observed in the absence of the inhibitor. Inhibitors include those that block or modulate the biological or immunological activity of DPP. Inhibitors of DPP may include, but are not limited to, proteins, nucleic acids, carbohydrates, or any other molecules that bind to DPP.

“Immune cells” refer to any cell that is part of the immune system and helps the body fight infections and other diseases. Immune cells develop from stem cells in the bone marrow and become different types of white blood cells. These include neutrophils, eosinophils, basophils, mast cells, monocytes, macrophages, dendritic cells, natural killer cells, and lymphocytes (B cells and T cells).

“Natural Killer” or “NK” cells refer to a type of immune cell—large, granular lymphocytes with the central role of killing the virus-infected and malignantly transformed cells, without prior sensitization. A natural killer cell is a type of white blood cell that comprises part of the innate immune system. Natural Killer cells are lymphocytes in the same family as T and B cells.

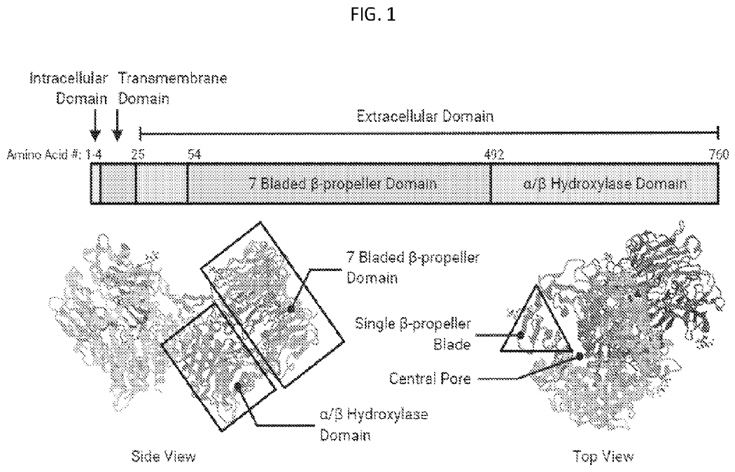

FAP is 760 amino acids long with residues 1-4 composing the intracellular domain, 5-25 composing the transmembrane domain and 26-760 composing the extracellular domain. APCE results from post translational cleavage and is thus the extracellular portion of FAP, residues 24-760 (K. N. Lee et al., 2006). Kathleen Aertgeerst (Aertgeerts et al., 2005) was the first to obtain a high-resolution crystalline structure of FAP. FAP's secondary structure consists of two domains, as shown in FIG. 1. FIG. 1 shows a schematic diagram of FAP domain structure (top) and ribbon models (bottom) depicting the FAP dimer. The seven-bladed β-propeller domain, a/P hydroxylase domain and β-propeller blade are highlighted.

Residues 54-492 comprise the B-propeller domain while residues 27-53 and 493-760 comprise the a/B-hydrolase domain. The B-propeller domain can be further broken down into eight blades surrounding a central pore of approximately 27 angstroms in length and 14 angstroms in width. Each blade is comprised of three or four anti-parallel B-sheets. The hydroxylase domain contains the catalytic triad while the B-propeller domain is believed to serve as filter so selectively permit peptides into the catalytic domain. The B-propeller domain is also thought to serve as the scaffolding region of FAP as certain B-sheets are the site for homodimerization, heterodimerization with DPPIV or interaction with other cell surface molecules such as integrins.

FAP's catalytic triad is located at the interface of the B-propeller domain and the a/B-hydroxylase domain. The catalytic triad is accessible via the pore formed by the B-propeller domain or via the cavity between FAP's two domains. The cavity offers greater access to substrates as its 24-angstrom width makes it wider than the pore.

Since FAP shares such sequence homology with DPPIV, attempts have been made to identify the structural differences that allot FAP its additional endopeptidase activity. Both enzymes' dipeptidyl peptidase activities are dependent on conserved amino acids Glu205, Glu206 and Tyrosine662, which render the catalytic binding site negatively charged and allow for binding of the positively charged amino group at the N-terminus of peptides. Two more conserved peptides, Arg125 and Asn710 (numbering based on DPPIV) are required for DPPIV activity because they bind to and stabilize the carbonyl oxygen of the P2 amino acid in the substrate (Aertgeerts et al., 2005). Aertgeerts et al. discovered that where DPPIV contains an Asp (663) FAP contains Ala (657) and this difference is responsible for FAP's endopeptidase activity

FAP requires both dimerization and glycosylation to be functionally active (Pifleiro-Sanchez et al., 1997; Sun et al., 2002) FAP is can homodimerize or heterodimerize with DPPIV (Ghersi et al., 2006). Hence, original work identified FAP as having two subunits, a and B, until further studies revealed FAP B was in fact DPPIV. FAP can also bind to B-integrins. It is believed integrins provide localization to invadopodia in cells grown on a collagenous matrix. Thus, it was assumed that this heterodimer functions to enhance extra cellular matrix degradation and invasion (Mueller et al., 1999). Furthermore, since FAP has a short cytoplasmic domain, integrins may serve as the liaison for FAP's effects on intracellular signaling. FRET data also suggests FAP can colocalize with urokinase plasminogen activator receptor (uPAR) (Artym et al., 2002). Given that uPAR and FAP both play a role in tissue organization, their biological association seems reasonable.

FAP has five potential N-linked glycosylation sites on asparagine residues 49, 92, 227, 314 and 679. Four are in the β-propeller domain and one is in the hydroxylase domain. Sun et al. found that glycosylation was necessary for FAP endopeptidase activity (Sun et al., 2002).

FAP Genetics

The human FAP gene is located on chromosome 2q23. It spans approximately 73 kb and contains 26 exons. FAP continues to share remarkable homogeneity with DPPIV even at the gene level. DPPIV is located on chromosome 2q24.3, spans 70 kb and contains 26 exons. Hence some believe FAP arose from a DPPIV duplication. FAP has been identified in several other species including mouse (Cheng et al., 2002; Joachim Niedermeyer et al., 1998) and xenopus (Brown et al., 1996). The mouse FAP gene is highly similar to human, located on chromosome 2, spanning 60 kb and containing 26 exons. Thus, mouse models can offer useful preclinical models to study FAP.

In 2010, Jiping Zhang identified the human and mouse promoter region of FAP. It is a 245-bp fragment surrounding the transcription start site. It contains early growth response-1 (EGR1), HOXA4, and E2F1 transcription binding sites. Of these three binding sites, EGR1 appeared to be the most important transcription factor for driving FAP expression (J. Zhang et al., 2010).

Splice Variants

Like many proteins, FAP is known to have splice variants. Leslie Goldstein identified alternatively spliced FAP that forms a truncated protein in the melanoma cell line LOX. This variant is generated by an out-of-frame deletion of exonic region spanning 1223 bps. This region encodes part of the cytoplasmic tail, transmembrane and portions of the proximal and central extracellular domains. Sequence analysis of this alternatively spliced FAP variant predicts it to be entirely cytoplasmic. It is currently unknown if this splice variant has catalytic activity (L A Goldstein & Chen, 2000). Additionally, three FAP splice variants have been identified in mouse embryonic tissues. All three variants encode the entire protein, including the catalytic triad, but lack part of the extracellular domain near the transmembrane domain (Joachim Niedermeyer et al., 1997). Interestingly, there are no reports of DPPIV alternative splicing events.

Induction

Little is known about the physiologic regulators of FAP expression, however in vitro studies have offered some insights. In vitro, FAP can be induced in leptomeningeal fibroblasts by TGFB, TPA (tetradecanoyl phorbol-13-acetate), retinol or retinoic acid (Rettig et al., 1994). TGF-B and IL1-B alone and synergistically induce FAP expression in mouse fibroblasts (H. Chen et al., 2009a). UVA and UVB can induce FAP expression in fibroblasts, melanocytes and primary melanoma cells. Furthermore, primary melanoma cell line media, but not metastatic melanoma media, can induce FAP expression in fibroblasts without UVR exposure (Wister et al., 2011). In human aortic smooth muscle cells FAP is induced by TNFa. This study also demonstrated conditioned media from peripheral blood-derived macrophages induced FAP expression in aortic smooth muscle cells and that this effect was abolished upon addition of TNFa inhibitors. Thus, they infer that TNFa released from immune cells, in this instance macrophages, is responsible for induction of FAP.

In vitro studies investing the role of FAP expression in ovarian cancer found that FAP is induced in ovarian fibroblasts by exposure to conditioned media from an ovarian cell line HO-8910PM or upon adhesion to type I collagen (H. Chen et al., 2009a; Kennedy et al., 2009). Once elevated, FAP promotes proliferation, adhesion and migration of metastatic ovarian cancer cell and ovarian cancer associated fibroblasts (H. Chen et al., 2009a; Kennedy et al., 2009; Lai et al., 2012).

One study in glial tumors demonstrates that FAP is increased upon cellular differentiation. In this study glioma stem-like cells from glioblastoma were isolated, then differentiation was induced in vitro by long term culture with basic fibroblast growth factor and epidermal growth factor. After differentiation, FAP was upregulated 40-fold, yet DPPIV remained unchanged.

A recent study points to micro-RNAs as regulators of FAP expression. Peng Ruan demonstrated that miR-30a-5p downregulated FAP expression in oral cavity cancer cells, resulting in decreased cell propagation, migration and invasion, consistent with previous reports of FAP function (Ruan et al., 2018). Many other factors have been shown to influence FAP expression in a context-dependent manner and will be addressed herein.

FAP in Development and Health

Most of what is known about FAP's role during development is from studies on frogs and mice. Amphibian metamorphosis, the transformation of the larva to a miniature adult, involves complex developmental programs that requires physiologic and morphological changes regulated by thyroid hormone. Most of the thyroid hormone regulated tissue remodeling, including tail resorption, involves cell death. Donald Brown's group conducted a time course gene expression screen to identified thyroid hormone upregulated and downregulated genes responsible for tail resorption. They identified a set of “direct response genes” that are activated 2-4 hours after exposure to thyroid hormone and peak at 12 hours, and a set of “delayed response genes” that were maximally upregulated 24 hours after thyroid hormone induction. They proposed that the direct response genes were responsible for inducing the delayed response genes. One of the eight genes identified in the delayed response genes was FAP, in addition to two other proteases, collagenase-3 and peptidase R. Expression of FAP at this stage of metamorphosis was not exclusive to the tail, and it was proposed that this is because tissue remodeling is not limited to the tail but is essential for many other organs during metamorphosis (Brown et al., 1996; Kanamori & Brown, 1996). From here we can presume that FAP is expressed in addition with collagenase-3 and peptidase E to remodel the extracellular matrix to allow for tissue remodeling.

FAP deficient mice (FAP−/−) are viable and display no overt developmental defects (J Niedermeyer et al., 2000). Joachim Neidermeyer et al replaced the FAP gene with a B-galactosidase that was under regulation of the FAP promoter. After 11.5 days post conception, they found B-galactosidase expression in somites, myotubes and perichondral mesenchyme from the cartilage primordia. At day 16.5 post conception scattered developing intercostal muscle fibers expressed B-galactosidase but B-galactosidase subsequently repressed after birth. The replacement of FAP with B-galactosidase resulted in no obvious phenotypes, suggesting that FAP is associated with tissue remodeling but not necessary in embryonic development. The upregulation of compensatory proteolytic enzymes may contribute to normal development in FAP deficient models (J Niedermeyer et al., 2001).

While FAP has been traditionally considered absent from adult tissues, a more systemic approach to FAP expression profiling in mice with extra-chromosomal luciferase under the control of the FAP promoter suggests that low basal levels of FAP expression might be found in many tissues, including muscle, bone marrow, adipose, skin, and pancreas (Roberts et al., 2013).

FAP has also been identified in human plasma from non-diseased individuals, although the source of this circulating FAP is unknown (Keane et al., 2014). There is one context in which FAP expression in adult tissues is universally accepted—wound healing. Consistent with FAP's tissue remodeling role in embryologic development, FAP is known to be strongly induced in the process of scar formation. Immunohistological evaluation of six human surgical incision wounds demonstrated all six had extensive FAP expression (Garin-Chesa et al., 1990).

FAP in Non-Oncological Diseases

FAP has been linked to multiple human pathologies including fibrosis, arthritis, atherosclerosis, autoimmune diseases, metabolic diseases and cancer. In most instances, FAP is associated with progression and heightened severity of the disease, but there are some conflicting reports.

Fibrosis

Given FAP's role in tissue remodeling and expression on activated fibroblasts of scarring tissue, it is unsurprising that FAP expression is associated with diseases of uncontrolled scarring, known as fibrosis. FAP has been reported elevated in fibrotic conditions involving the liver, lung and colon. Liver fibrosis can ultimately lead to liver failure, a condition termed cirrhosis. Initiation of liver fibrosis is believed to be chronic injury from etiologies such as a viral hepatitis infection, non-alcoholic fatty-liver disease or alcoholism. With chronic liver injury, hepatic stellate cells, which are normally quiescent and function to store vitamin A, become activated and begin producing the extracellular matrix responsible for hepatic scarring. Activated hepatic stellate cells take on a more myofibroblast like phenotype and express a smooth muscle actin (aSMA), glial fibrillary acidic protein (GFAP), and FAP (M. T. Levy et al., 1999). Intrahepatic expression of FAP, but not GFAP or aSMA, correlated with degree of liver fibrosis in patients with viral hepatitis C infections (M. Levy et al., 2002). FAP activity was 14-18 fold greater in cirrhotic livers compared to healthy livers and circulating FAP was almost doubled in the presence of alcoholic cirrhosis (Keane et al., 2014). Shirley Uitte de Willige showed that the concentration and activity of circulating FAP was significantly increased in patients with liver cirrhosis and that these increased levels correlated with increased cleavage of a-2 anti-plasmin. N-terminal cleaved a-2 anti-plasmin is a more potent inhibitor of fibrinolysis than its uncleaved protein and thus they propose that increased circulating FAP may be responsible for the hemostasis related bleeding and thrombotic events associated with liver cirrhosis. Interestingly, FAP levels normalized with successful liver transplant (Uitte de Willige et al., 2017; Uitte De Willige et al., 2013). A study by KH Williams demonstrates that low levels of circulating FAP can be used clinically to rule out clinically significant liver fibrosis in patients with non-alcoholic fatty liver disease (Williams et al., 2015).

Idiopathic pulmonary fibrosis (IPF) is another disease of uncontrolled fibrosis, this time affecting the lung. This chronic lung disease is characterized by excessive fibrosis of the lung interstitium with no clear etiology or successful treatments. FAP is specifically upregulated in fibroblastic foci and the fibroblastic interstitium of patients with IPF but not in adjacent normal tissue, lung tissue from healthy individuals or lung tissues from patients with centri-acinar emphysema (Acharya et al., 2006). FAP is also upregulated in mouse models of IPF and levels of FAP expression in the lungs correlate to the severity of IPF (Wenlong et al., 2015).

Interestingly, IPF is exacerbated in FAP deficient mice, and restoration of FAP to FAP deficient mice significantly reduced lung collagen content. This finding therefore suggests that FAP plays a protective role in the lung and functions to combat fibrosis by promoting collagen clearance and matrix degradation (M.-H. Fan et al., 2016). However, these surprising findings are contradicted by a study demonstrating that a nonspecific FAP inhibitor (PT-100, Val-boro-pro, Talabostat, BXCL-701) had anti-fibrotic effects. In in vivo models of IPF, an FAP inhibitor slowed disease and reduced fibrosis (Egger et al., 2017). While the specific roles of FAP in IPF remain uncertain, its involvement in the disease is undisputed.

Other pathologies in which extensive fibrosis is correlated with upregulated FAP expression include keloid formation and Crohn's disease. Keloid scars are benign, fibroproliferative dermal lesions of unknown etiology and commonly occur following surgical resection. Keloids progress in a manner dependent on increased deposition of extracellular matrix and invasion into surrounding healthy skin. One study demonstrated that fibroblasts derived from keloid skin samples had elevated expression of FAP, increased invasiveness and enhanced extracellular matrix deposition when compared to fibroblasts derived from control skin samples. Selective inhibition of FAP/DPPIV resulted in decreased invasion but had no effect on other phenotypes such as increased extracellular matrix deposition or expression of pro-inflammatory cytokines (Dienus et al., 2010).

Crohn's disease is an autoimmune condition resulting in chronic gut inflammation that can be complicated by intestinal fibrosis and stricture formation. One study identified FAP to be overexpressed in uninflamed strictures compared to non-structured colonic regions in biopsies taken from Crohn's Disease patients. FAP was not overexpressed in colonic biopsies taken from healthy individuals or individuals with ulcerative colitis, a different inflammatory bowel disease. FAP expression was increased in myofibroblasts derived from structured lesions upon exposure to TNFa and TGF-B, but that this was not true for myofibroblasts derived from non-strictured lesions (Rovedatti et al., 2011). These results imply that FAP cannot be induced in any fibroblast upon exposure to inducing factors, but some reprogramming of cells prior to pro-FAP factors is required.

Arthritis

Arthritis is a term used to mean any disorder that affects the joints. The two most common forms of arthritis are osteoarthritis and rheumatoid arthritis. Osteoarthritis is also known as degenerative joint disease and occurs with aging. Rheumatoid arthritis is an autoimmune condition. The investigation of FAP in arthritis was sparked when a phase I clinical trial of radiolabeled anti-FAP antibody demonstrated minor antibody uptake in the knees and shoulders of patients who lacked clinical symptoms of arthritis (Scott et al., 2003).

Osteoarthritis is characterized by degradation of joint cartilage. Joint cartilage is largely composed of proteoglycans, collagen and chondrocytes, the cells responsible for cartilage maintenance. Milner et al. were the first to demonstrate that chondrocytes expressed FAP and that chondrocyte FAP expression was elevated in patients with osteoarthritis. They demonstrated that chondrocytes increased FAP expression in response to cartilage resorption signaling cytokines, IL-1 and oncostatin M, and that this induction of FAP correlated with increased collagen breakdown in vitro. FAP expression was elevated in mRNA extracted from collagen derived from osteoarthritis patients compared to cartilage of normal patients. All osteoarthritis patients expressed FAP in the superficial zone of cartilage and on chondrocyte membranes by immunohistochemistry (Milner et al., 2006). Thus, this paper suggests FAP is involved in cartilage degradation associated with osteoarthritis. Rheumatoid arthritis is an autoimmune chronic inflammatory disease of unknown etiology and is characterized by chronic inflammation of the joint capsule's synovial membrane. This chronic inflammation ultimately destroys the underlying cartilage and bone. Activated fibroblast-like synoviocytes (FLS) line the synovial membrane and are a prominent cell type responsible for inflammation and joint destruction. One study identified FAP expression in synovial samples taken from both rheumatoid arthritis and osteoarthritis patients. However, FAP expression was greater in samples taken from refractory rheumatoid arthritis patients in comparison to end stage osteoarthritis patients (Bauer et al., 2006). While the association of FAP and arthritis was clear, the role of FAP in arthritic diseases remained elusive. Ospelt et al. showed that inhibition of FAP/DPPIV worsened arthritic lesions in vivo models. Treatment of animals with a FAP/DPPIV inhibitor increased synovial expression of MMP-1 and MMP-3 and increased collagen destruction (Ospelt et al., 2010). However this group also demonstrated that DPPIV knockout mice had worsened arthritic lesions (Busso et al., 2005) and as such the pro-arthritic effects of this inhibitor can be attributed to its effects on DPPIV. In 2015, Waldele et al. developed a transgenic mouse model of chronic inflammatory arthritis that lacked FAP. In this model, FAP deficiency led to decreased cartilage degradation, even though the amount of inflammation and bone degradation was unchanged. They demonstrated that synovial fibroblasts derived from FAP deficient mice had decreased ability to adhere to cartilage (Waldele et al., 2015). Laverman et al demonstrated that the use of radiolabeled anti-FAP antibodies accurately represented synovial inflammation severity in mouse models of rheumatoid arthritis (Laverman et al., 2015), suggesting the association between FAP and arthritis could be exploited for clinical benefit.

Cardiovascular Disease

Many pathologies fall under the term cardiovascular disease, including atherosclerosis and myocardial infarction. Atherosclerosis is characterized by subendothelial accumulation of fatty substances, called plaques, that lead to inflammation and tissue remodeling. These atheromatous plaques can rupture and cause myocardial infarction, stroke or sudden cardiac death. There are two types of atheromatous plaques—thin cap and thick cap. One study identified overexpression of FAP in human aortic smooth muscle cells of thin cap atheromas in human biopsies. FAP was induced by TNFa released from macrophages and FAP levels correlated with macrophage infiltration. In vitro studies then demonstrated that once FAP is expressed, it cleaves the type I collagen present in the cap and renders the plaque rupture-prone. Treatment with an anti-FAP antibody resulted in decreased collagen cleavage (Brokopp et al., 2011).

Several studies investigated the levels of soluble FAP in the plasma of patients with various atherosclerosis related diseases. These studies showed levels of soluble FAP were unaffected by conditions such as ischemic stroke and peripheral artery disease, but that FAP levels were decreased in patients with coronary heart disease and acute coronary syndrome. In acute coronary syndrome, decreased soluble FAP levels correlated with worse clinical outcomes, as patients with FAP levels in the first quartile had a 3-fold higher risk of death. Furthermore, investigators found that fluctuations in FAP levels were not permanent and that over time, levels returned to that of the control population (Tillmanns et al., 2013; Uitte De Willige et al., 2015).

One study demonstrated that in rats, cardiac expression of FAP increased after induction of a myocardial infarction (MI). This was especially true for the myofibroblasts in the peri-infarct area. Peak FAP expression was seen 7 days post MI. These findings were confirmed in human cardiac specimens, with FAP+ fibroblasts being abundant in ischemic tissue post-MI but absent in healthy control cardiac specimens (Tillmanns et al., 2015). In plasma samples obtained from patients post ST-elevation myocardial infarction, FAP concentrations were inversely related to established cardiac enzymes, CK and CPR. Greater declines of FAP from admission to 5 days post admissions were associated with increased myocardial damage and inflammation (Tillmanns et al., 2017).

Metabolic Disease

Given the recent discovery that FAP cleaves and inactivates the hormone FGF21 (Dunshee et al., 2016; Zhen et al., 2016), the role of FAP in metabolic diseases has just started to be investigated. FGF21 is a stress-induced hormone with potent anti-obesity, insulin-sensitizing and hepatoprotective properties. One study demonstrated that administration of talabostat, a nonspecific inhibitor of FAP, to mice with diet induced obesity had significantly reduced body weight, food consumption, adiposity and cholesterol with simultaneously increased energy expenditure, glucose tolerance and insulin sensitivity (Sanchez-Garrido et al., 2016). This effect was abrogated in FGF21 deficient mice, thus confirming that the metabolic benefits of FAP inhibition can be attributed to increased circulating FGF21.

FAP in Cancer

While FAP expression in normal tissues is usually low or undetectable, it is overexpressed in many cancers, including 90% of carcinomas. FAP is known to be overexpressed in breast, colorectal, pancreatic, lung, bladder, ovarian and other cancers. In these cancers, FAP is usually heavily expressed in the stroma, and has thus become a universal marker of cancer-associated fibroblasts (CAFs). While the presence of FAP in malignant tissues is undisputed, the role of FAP biologically and its impact on disease prognosis has been inconsistent throughout the literature.

Breast Cancer

One of the earliest publications about FAP identified FAP overexpression in the stroma of breast epithelial tumors and focal expression in some of the samples of fibrocystic disease while FAP was absent from normal breast tissue or benign breast tumors (Garin-Chesa et al., 1990). One study identified increased FAP expression in ductal carcinoma in sills that would progress to ductal carcinoma versus DCIS that would not progress. This suggests pathologists could utilize FAP to improve clinical prediction of progression and fine tune treatment recommendations (Hua et al., 2011). While most studies confirmed the existence of FAP in the stroma surrounding breast cancer cells, one study identified FAP expression in the breast cancer cell lines themselves (Goodman et al., 2003). Reports on the impact of FAP expression on disease prognosis are inconsistent. FAP expression in stromal tumor components is greater in invasive lobular carcinoma than invasive carcinoma of no special type (C. K. Park et al., 2016).

In invasive ductal carcinoma, elevated FAP was associated with high histological tumor grade as well as an inflammatory- and adipose-type stroma but not desmoplastic, sclerotic or normal-like stroma (Jung et al., 2015; S. Y. Park et al., 2015). In phyllodes tumors, a benign breast tumor that has rare malignant transformation, increased FAP mRNA levels were associated with malignant transformation, suggesting that FAP can be utilized to determine the malignant potential of these tumors (Gong et al., 2014), similar to its prognostic value for DCIS. The prognostic value of FAP in breast cancers of all subtypes is controversial, with some studies demonstrating that elevated FAP is associated with worse survival (Jia et al., 2014), and others associating elevated FAP is associated with improved survival (Ariga et al., 2001).

Colorectal Cancer

In human colon cancer specimens, FAP expression has been identified in both cancer cells and in adjacent stromal cells, including myofibroblasts, fibroblasts and endothelial cells (Iwasa et al., 2003). FAP staining intensity was inversely correlated with patient tumor stage and xenograft tumor size. Elevated FAP expression noted early in tumor development (Henry et al., 2007). These data suggested that stromal FAP may play a role in the development of colorectal tumors. Perhaps in accordance with this finding, human colorectal specimens were noted to have elevated FAP at the tumor front versus the tumor center, suggesting the role of FAP in tumor invasion. This study also found that FAP was more likely to be expressed in the center of tumors post-radiotherapy, perhaps due to the tissue remodeling required after radiation inflicted damage (Wikberg et al., 2013). In human samples, high FAP was associated with increased depth of invasion, lymph node metastasis, higher grade and stage and worse overall survival. (Henry et al., 2007; Iwasa et al., 2003; Wikberg et al., 2013; X. Yang et al., 2016). Tumoral FAP expression also correlated with a shift in immune cell populations. Elevated FAP was associated with reduced CD3+ cells but increased CD11b+ cells (X. Yang et al., 2016).

Pancreatic Cancer

Ninety percent of pancreatic ductal adenocarcinomas (PDAC) demonstrate FAP staining. FAP expression has been identified in both the tumor stromal compartment as well as PDAC tumor cells and pancreatic cancer cell lines (M. Shi et al., 2012). FAP expression in stromal tissue is greatest at the tumor front. Low FAP expression is associated with increased pancreatic fibrosis while high FAP expression is associated with increased risk of lymph node metastasis, tumor recurrence and death (Cohen et al., 2008). In vivo studies utilizing an endogenous KPC PDAC tumor mouse model in FAP knockout mice demonstrated that FAP deficiency delays tumor onset and prolongs survival, increases tumor necrosis and impedes distant metastasis (Lo et al., 2017). FAP expression was identified in both the malignant lesions as well as the premalignant lesions, termed PanINs, of KPC mice (Feig et al., 2013). Many more studies have confirmed the association between elevated FAP and worse clinical outcomes (Lo et al., 2017; M. Shi et al., 2012). Elevated FAP expression was positively correlated with patient age, tumor size, fibrotic foci, perineural invasion and pore survival (M. Shi et al., 2012). However, some studies have found that FAP expression was correlated with improved clinical outcomes (Kawase et al., 2015; H. Park et al., 2017).

Gastric Cancer

Gastric cancer consists primarily of two types: intestinal-type and diffuse-type. Both types express FAP, however intestinal-type does so to a larger degree. Unlike other cancers, in gastric cancer the majority of FAP expression is localized to the gastric carcinoma cells and is only weakly expressed in stromal and endothelial cells (Mori et al., 2004; Okada et al., 2003). In human tissues high FAP expression is correlated with high grade, lymph node metastasis, peritoneal invasion and worse overall survival (Hu et al., 2017; X. Wen et al., 2017). Models of gastric cancer demonstrated that co-culture of gastric cancer cells with FAP expressing fibroblasts resulting in increased proliferation and migration in vitro and increased tumor growth and resistance to anti-PD-1 therapy in vivo (X. Wen et al., 2017). One gastric cancer model study showed that administration of polyphyllin, a plant derived compound, decreased CAF proliferation in vitro and decreased tumor growth in vivo via downregulation of FAP (Dong et al., 2018).

Brain Cancer

Original work studying FAP suggested primary brain tumors did not express FAP but metastatic carcinoma lesions did (Garin-Chesa et al., 1990; Rettig et al., 1986). Future work would go on to challenge this concept and demonstrate that FAP is expressed in high grade lesions. Grade III and IV human astrocytic tumors express FAP mRNA, while Grade II and nonmalignant lesions do not (Stremenova et al., 2007). In glial tumors, there is increasing FAP mRNA expression as grade increases and within the grade IV subtypes, glial sarcomas have significantly more FAP expression than glioblastomas (Matrasova et al., 2017; Mentlein et al., 2011; Mikheeva et al., 2010). FAP expression in gliomas is correlated with worse overall survival, however this can be attributed to the fact that the most malignant gliomas are associated with increase FAP expression (Busek et al., 2016).

Ovarian Cancer

FAP expression was detected in 97% of ovarian cancers, but not in normal ovarian tissue, benign ovarian tumors or ovarian tumors of low malignant potential (Garin-Chesa et al., 1990; Rettig et al., 1986; Yuan Zhang et al., 2011). While FAP is not believed to be expressed in ovarian epithelial cancer cells, one study demonstrated FAP knock down in SKOV3 ovarian cancer cells lines resulted in decrease decreased FAP expression in surrounding fibroblasts, decreased tumor growth, volume and proliferation (Lai et al., 2012). In a complementary experiments, SKOV3 lines transfected with FAP to over-express FAP stably had increased tumor growth, proliferation and invasion in vitro (L. Yang et al., 2013). In human studies, an elevated level of FAP in peritoneal or pleural effusions from epithelial ovarian cancer patients correlated with decreased survival rates (M.-Z. Zhang et al., 2007). Strong stromal staining for FAP and DPPIV by IHC and mRNA levels by in-situ hybridization were associated with higher stage and increased metastasis to the lymph nodes and the omentum. By contrast, no significant correlation was detected among FAP/DPPIV protein/mRNA levels and patient age, histological grade or tumor type. Furthermore, elevated FAP levels, but not DPPIV levels, were associated with shorter disease-free survival (M. Zhang et al., 2015; Yuan Zhang et al., 2011).

Myeloma

Multiple myeloma is a hematologic malignancy that affects plasma cells. Unique to myeloma is the clinical feature of osteolytic bone disease whereby increased osteoclast activity and decreased osteoblast numbers results in bone break down, which has been hypothesized as a means for myeloma cell expansion within the bone marrow. While FAP is not expressed in myeloma cells, it was identified as one of 28 genes selectively upregulated in osteoclasts upon coculture with myeloma cells, while the other related serine protease levels were unchanged. In multiple myeloma patient bone marrow biopsies, FAP was expressed by osteoclasts, osteoblasts and osteocytes along the bone surface and in fibrotic regions. In the same study FAP knockdown in osteoclasts led to decreased myeloma cell survival in coculture. In vivo myeloma studies demonstrated FAP mRNA was upregulated more than 40-fold in the bones of mice inoculated with myeloma cell lines compared to uninoculated mice (Ge et al., 2006). Further work by this group demonstrated that the addition of talabostat to cocultures of patient-derived osteoclast and myeloma cells resulted in talabostat concentration-dependent decreased myeloma cell proliferation. In vivo application of talabostat in SCID myeloma models reduced osteoclast activity, bone resorption and tumor burden (Pennisi et al., 2009).

Melanoma

Even though the earliest descriptions of FAP were within the context of melanoma, the role of FAP in melanoma is still controversial. Huber et al. systematically determined the expression pattern and enzymatic activity of FAP in both stromal cells and melanocytes in a series of melanocytic lesions ranging from benign melanocytic nevi, commonly referred to as moles, to metastatic melanoma. FAP is expressed in the stromal fibroblasts of all melanocytic tumors, including benign, premalignant and malignant, however, FAP expression was absent in fibroblasts from normal adult skin. While FAP is expressed in the stroma of benign melanocytic tumors, its expression increases in the stroma of malignant and metastatic lesions. This study identified FAP expression on the surface of melanocytes in 30% of benign melanocytic nevi, while melanocytes from primary and metastatic melanoma lesions had no detectable levels of FAP expression (Huber et al., 2003). However, Aoyama et al. demonstrated FAP expression by melanoma cell lines correlated with an increasingly invasive phenotype (Aoyama & Chen, 1990). In these melanoma cell lines, FAP was found to be localized to invadopodia, thus promoting matrix degradation and cellular invasion (Monsky et al., 1994; Piñeiro-Sánchez et al., 1997).

In summary, FAP expression's impact on clinical factors such as tumor type and clinical outcomes is highly variable and depends on cancer type, histological type, tumor localization and specific cellular expression (stromal vs. malignant cells). A recent meta-analysis assessed the prognostic value of FAP in solid tumors by performing a global analysis of 15 studies and concluded that FAP overexpression in tumor tissues displayed significant associations with poor overall survival and tumor progression. Subgroup analysis revealed the correlation between FAP overexpression and poor overall survival and lymph node metastasis was more pronounced in patients with FAP expression in tumor cells (F. Liu et al., 2015).

Functional Roles of FAP in Cancer

Given the extensive expression of FAP in many cancer types, the pro-tumorigenic or anti-tumorigenic role of FAP has been thoroughly investigated. To date, FAP has been reported to influence tumor growth via multiple mechanisms including promoting proliferation, invasion, angiogenesis, epithelial-to-mesenchymal transition, stem cell promotion, immunosuppression and drug resistance.

Proliferation, Migration, and Invasion

Perhaps the most consistent finding in the literature is the effect of FAP on cell proliferation, migration and invasion, all of which promote tumor growth. It has been demonstrated FAP can promote invasion of endothelial cells, melanoma cells, ovarian cancer cell lines, oral cancer cells, and fibroblasts (Ghersi et al., 2006; Kennedy et al., 2009; Monsky et al., 1994; Ruan et al., 2018; Wäster et al., 2011). How FAP promotes proliferation and migration is still contested. There are two main hypotheses. The first is the indirect hypothesis: FAP regulates extracellular matrix remodeling and the changes to the matrix are then responsible for increased capability of cell growth. Even proponents of this hypothesis, however, dispute if FAPs regulation of the extracellular matrix can be attributed to its enzymatic activity or if it is due to FAP independent of its enzymatic activity. The second hypothesis is a direct hypothesis: FAP expression alters intracellular signaling pathways, which in turn affect cell cycle and proliferation pathways to promote cell growth.

The indirect hypothesis has been supported by many studies. Some of the earliest work on FAP demonstrated its localization to the tips of invadopodia in melanoma cells and associated increased extracellular matrix degradation and invasion (Monsky et al., 1994; Nakahara et al., 1996). It is believed that a3B1 integrin is necessary for appropriate localization of FAP to invadopodia (Mueller et al., 1999). The role of a3B1 integrin in FAP induced proliferation and migration was further investigated in a study where inhibition of a3B1 integrin attenuated the FAP induced proliferation invasion and migration in ovarian cancer cell lines (W. Yang et al., 2013). This then implies that it is not the enzymatic activity of FAP that is causing these phenotypic changes but rather the association of FAP with a3B1 integrin. These findings are further supported by evidence that breast cancer cell overexpressing wild type and catalytically inactive FAP display increased extracellular matrix degradation and invasion on type I collagen gels (Y. Huang et al., 2011). One study generated doxycycline-inducible FAP overexpressing fibroblasts and cocultured them with pancreatic ductal adenocarcinoma cells to assess the effects of FAP on extracellular matrix and malignant cell phenotype. The authors found that FAP expressing fibroblasts induced architectural and compositional changes to the extracellular matrix that allowed for enhanced velocity of pancreatic cancer cell migration. In agreement with previous literature, this study concluded that enhanced migratory phenotype is mediated by B1 integrin as addition of an integrin inhibitor reversed the phenotypic changes (H.-O. Lee et al., 2011). However, in the same study the addition of an FAP inhibitor led to extracellular matrix disorganization that impeded pancreatic cancer cell invasion, thus implying that the enzymatic activity is also required for extracellular matrix remodeling. The role of FAP's enzymatic activity in extracellular matrix remodeling has been investigated in other studies as well. FAP knock out mice had accumulation of intermediate-sized collagen fragments in lung tissue in compared to wild type mice. This observation was recapitulated when wild type mice were treated with an FAP inhibitor. In another study focusing on melanoma, ultraviolet radiation-induced FAP expression in fibroblasts and these fibroblasts displayed greater migratory capacity that was associated with increased collagenase I activity (Waster et al., 2011).

The hypothesis that FAP has direct effects on intracellular proliferation and cell cycle signaling pathways is also supported by many studies. Alterations of FAP expression induces changes in common cell signaling pathways or gene expression. SiRNA knockdown of FAP in tumor-associated fibroblasts derived from ovarian cancers inhibited cell proliferation, induced cell cycle arrest and decreased the expression of stem cell associated genes. (Lai et al., 2012). In a squamous cell lung carcinoma cell line, FAP overexpression promoted proliferation, motility and invasion while simultaneously upregulating PI3K/Akt and SHH/Gli1 signaling (Jia et al., 2017). The importance of these signaling pathways in promoting cellular proliferation and invasion was confirmed when inhibition of SHH and PI3K abrogated the phenotype. This same group studied the effects of FAP on cell signaling in breast cancer lines. Interestingly, the overexpression of FAP in breast cancer lines resulted in decreased motility. Overexpression of FAP reduced FAK phosphorylation, and the reduction in FAK activity caused the decreased motility phenotype (Jia et al., 2014). In oral squamous cell carcinoma, knockdown of FAP resulted in decreased growth and metastasis in vitro and in vivo. Silencing FAP expression reduced the activation of pRb and oncogenic cell-cycle regulators including CCNE1, E2F1, and c-Myc, but elevated the expression of tumor suppressors such as p27 and p21. Furthermore, FAP silencing significantly decreased the expression of phosphorylated PI3K, AKT, MEK1/2, ERK1/2, and GSK3b, whereas total levels remained unchanged. These results suggested that FAP is an upstream regulator of the PTEN/PI3K/Akt and Ras-ERK signaling pathways in oral squamous cell carcinoma (H. Wang et al., 2014). One study focused on the effects of FAP expressing fibroblasts on pancreatic ductal adenocarcinoma cell lines, showing that coculture of PDAC lines with FAP+ fibroblasts resulted in increased phosphorylation of Rb in the cancer cells, leading to cell cycle progression and increased proliferation (Kawase et al., 2015). Both hypotheses have merit and are supported by the available evidence. FAP's effects on proliferation, motility and invasion could be a consequence of its extracellular matrix remodeling as well as its intracellular signaling, and could depend on both the enzymatic and non-enzymatic activities of FAP. Yang et al. demonstrated that in ovarian cancer cell lines, FAP-integrin dimer formation and FAP induced intracellular activation of Rac1 induced increased proliferation and migration; inhibition of either integrin or Rac1 reversed the phenotype (W. Yang et al., 2013). One can imagine a situation in which the docking of FAP to invadopodia by integrins serves two purposes. The first is to localize FAP to the leading edge of cellular invasion to allow to matrix remodeling and easier migration. The second is so that FAP can trigger intracellular signaling through integrins to promote invasion, migration and proliferation gene signaling. This complementary perspective of FAP signaling also implicates the need for FAP's enzymatic function and non-enzymatic function to promote the pro-tumorigenic phenotype.

Angiogenesis

In 2003, Aimes et al. discovered that human endothelial cells are capable of producing FAP and that FAP, like other serine proteases, has regulatory roles in microvascular endothelial cell reorganization and capillary morphogenesis (Aimes et al., 2003). In in vivo models, inoculation of SCID mice with FAP+ breast cancer cell lines resulted in faster growing, highly vascularized tumors even though these FAP+ cells did not have any proliferative advantage in vitro. Histological analysis of gastric cancer biopsies demonstrated that gastric cancers with high FAP expression also had increased micro-vessel density compared to gastric cancers with lower FAP expression (Gao et al., 2017). These findings were further validated by a study demonstrating that FAP knock out or pharmacologic inhibition of FAP resulted in decreased tumor growth and decreased tumor microvascular density in in vivo models of lung cancer and colon cancer (Santos et al., 2009). These data suggest that the enzymatic activity of FAP is responsible for increased angiogenesis. While FAP is not believed to be expressed by ovarian epithelial cancer cells, one study demonstrated that FAP knockdown in SKOV3 ovarian cancer cells lines led to decreased expression of VEGF and EGF, suggesting FAP's role in tumor angiogenesis (Lai et al., 2012). A recent study aimed at elucidating the differential functions of the endopeptidase and dipeptidyl peptidase activates of FAP demonstrated that FAP expression by human endothelial cells early in the stages of capillary tube formation, followed by subsequent abrogation of FAP expression once tubes had formed (Christiansen et al., 2013). These findings are further validated by a study that demonstrated FAP expression by the endothelial cells of capillaries, but not large blood vessels, in invasive ductal carcinoma in vivo. This FAP expression localized to the invadopodia of endothelial cells (Ghersi et al., 2006). observation suggests FAP promotes capillary growth and invasion into the extracellular matrix. FAP expressing stromal cells have been seen to localize around dysplastic blood vessels in glioblastoma (Busek et al., 2016). Additional studies have identified FAP expression on endothelial cells in the developing microvasculature in malignancies such as multiple myeloma, gastric carcinoma and breast cancer (Bhati et al., 2008; Ge et al., 2006; Okada et al., 2003).

It has been hypothesized that the proangiogenic qualities of FAP can be attributed to the dipeptidyl peptidase activity that it shares with DPPIV. One of the known substrates of FAP and DPPIV is neuropeptide Y, which, upon cleavage, becomes proangiogenic, promoting endothelial cell migration and tube formation on Matrigel (Zukowska et al., 2003). Another theory is that MMP-9, often co-expressed with FAP, is responsible for the angiogenic phenotypes of FAP expressing tumors, since MMP-9 is a known pro-angiogenic signaler (Vu et al., 1998). Interestingly, studies with catalytically inactive and active FAP demonstrate equal upregulation of MMP-9; therefore, this means of angiogenesis would not require FAP enzymatic activity (Y. Huang et al., 2011). The final way FAP may be involved in angiogenesis is indirectly, via its effect on extracellular matrix reorganization that may promote endothelial cell migration and neovascularization.

Epithelial-to-Mesenchymal Transition

Epithelial-to-mesenchymal transition (EMT) is defined as the acquisition of mesenchymal phenotype by malignant epithelial cells to allow for increased migration and invasion ultimately required for metastasis. In a technical paper warning against the use of anti-FAP antibodies as a means of isolating fibroblasts, it was demonstrated that many cell lines of epithelial origin expressed FAP in response to TGB-B induced EMT (Kahounovi et al., 2017). Oral squamous cell carcinoma cell lines with stable FAP knock down had decreased expression EMT-marker genes such as Snail, Slug, N-cadherin and Vimentin with E-cadherin expression increased (H. Wang et al., 2014).

While EMT is typically associated with invasive phenotypes of epithelial derived cancers, similar acquisition of mesenchymal phenotype has recently been observed in glial tumors, where the mesenchymal phenotype is associated with increased clinically aggressive tumors. TCGA analysis of glioblastomas demonstrated that 70% of mesenchymal glioblastomas had a 2-fold increase in FAP expression compared to other subtypes (Busek et al., 2016). A well-known regulator of EMT is the transcription factor TWIST1. In vitro glioma studies showed upregulation of TWIST1 in malignant glioma lines and association between TWIST1 and invasion. Subsequent studies demonstrated TWIST1 had pro-tumorigenic effects by inducing mesenchymal changes in glioma cell lines, including upregulation of FAP. This study went on to confirm TWIST1 and FAP were jointly upregulated in biopsies from the most aggressive glioblastoma tumors (Mikheeva et al., 2010).

Immunological Regulation

The effects of FAP on the immune system began to be investigated fairly recently. In 2009, Douglas Fearon's group published a study in Science that detailed the ability of FAP+ cells to suppress antitumor immunity. They generated transgenic murine models in which the fap gene contained a cassette encoding either GFP or diphtheria toxin receptor (DTR). Using GFP strains, they demonstrated FAP expression in both CD45+ and CD45− cells. Further sub phenotyping of these cells revealed the CD45+ population to resemble the CD11b+/classII+/Col1+/aSMA+fibrocyte and the CD45− population to resemble mesenchymal stem cells. Using the DTR strain they could ablate cells that express FAP by injecting diphtheria toxin. They then created immunogenic tumors by transfecting tumor cell lines with ovalbumin and vaccinated the mice with vaccinia virus expressing OVA. Prophylactic treatment of non-transgenic mice with the OVA vaccine successfully reduced tumor growth, demonstrating the efficacy of the vaccine. They then investigated the efficacy of OVA vaccine treatment with vaccine administration after tumor inoculation and found immediate tumor growth arrest upon FAP ablation for immunogenic tumors but not nonimmunogenic tumors. Surprisingly, they found no changes in T cell populations between FAP depleted and nondepleted mice, suggesting that the immunological impact of FAP is not T cell-mediated. Furthermore, reduction in tumor growth upon FAP ablation was reversed with anti-TNFa/anti-IFNy treatment. Therefore, this paper proposed that FAP suppresses production of TNFa and IFNy, or attenuates cellular responses to these cytokines. The relatively unchanged levels of these cytokines after FAP ablation would suggest the latter (Kraman et al., 2010). The same group utilized the DTR transgenic mice to investigate the role of FAP in PDAC. The found significantly reduced tumor growth upon ablation of FAP+ cells. However, contradictory to their previous findings, they found the reduced tumor growth was dependent on CD4+/CD8+ T cell activity and that FAP ablation enhanced the therapeutic benefits of anti-PD-1 and to a lesser extend anti-CTLA4 (Feig et al., 2013). This suggests that FAP contributes to the resistance of PDA to these immune checkpoints, at least in murine models. This is not the only study to imply that FAP serves as a resistance mechanism to immune checkpoints In vivo models of colorectal cancer demonstrated that co-injection of CRC cell lines with FAP+CAFs led to anti-PD-1 resistance (L. Chen et al., 2017). In vivo models of gastric cancer demonstrated a synergistic reduction in tumor growth of anti-PD-1 and an FAP inhibitor (X. Wen et al., 2017).

These findings stimulated investigations of the mechanism by which FAP may alter the intratumor immune milieu. One study demonstrated that FAP expressing cancer associated fibroblasts (CAFs) had a uniquely inflammatory gene expression signature in comparison to FAP-CAFs. Of the inflammatory genes upregulated by the FAP+CAFs, Ccl2 was most highly expressed (X. Yang et al., 2016). Furthermore, this study demonstrated that FAP's induction of CCL2 was independent of its enzymatic activity as addition of talabostat did not change the levels of these proteins. This group went onto to investigate the function of FAP+CAFs by co-injecting them with Hepa1-6 fibroma tumor lines. Tumors resulting from FAP+CAF containing mixtures had increased levels of PMN-MDSCs, M-MDSCs and macrophages, yet decreased IFNy+CD8+ T cells when compared to FAP-CAF cell mixtures. The showed that FAP+CAFs release CCL2, which in turn is recognized by the CCL2 receptor, CCR2, on circulating MDSCs, leading to their recruitment to tumor tissues. In Ccl2 knock out mice, tumor inoculation with FAP+CAFs lost their growth advantage over FAP-CAF tumors, and the resultant tumors had comparable levels of MDSCs. The ability of FAP+CAFs to produce CCL2, and its effects on MDSCs was also seen in a study investigating colorectal cancer (L. Chen et al., 2017). Other studies argue that a different cytokine, CXCL12, is responsible for the immunosuppressive environments associated with FAP+ fibroblasts. Feig et al. identified the primary source of tumor CXCL12 to be from FAP+CAFs. They then demonstrated that addition of an inhibitor to the CXCL12 receptor, CXCR4, reduced tumor growth in a T-cell dependent manner and enhanced the efficacy of anti-PD-1 but not anti-CTLA-4 (Feig et al., 2013). The ability of FAP+CAFs to secrete CXCL12 was confirmed by a study demonstrating that FAP+CAFs recognition of adenosine by the adenosine receptor A2B induces CXCL12 (Sorrentino et al., 2016).

The role of FAP in the immune system extends beyond its expression in cancer associated fibroblasts. There have been recent observations that FAP can be expressed by various immunological cells, including myeloid derived suppressor cells (MDSCs) and macrophages. Both healthy donor MDSCs and MDSCs derived from multiple myeloma patients expressed FAP on their cell membranes. When cultured in conditioned media from myeloma cell lines, the level of FAP expressed by multiple myeloma-derived MDSCs significantly increased. In vitro studies went on to demonstrate that when CD4+ T cells were cocultured with multiple myeloma derived-MDSCs, the CD4+ T cells exhibited decreased proliferation, increased senescence and increased differentiation into Th17 T cells. These changes were then reversed upon the addition of an FAP inhibitor. The phenotypic changes in the CD4+ T cells upon exposure to FAP were caused by activation of AKT; an AKT inhibitor rescued abnormal T cell differentiation and senescence. Another study detailed the presence of intra-tumoral FAP expressing F4/80hi/CCR2+/CD206+ M2 macrophages that induced immunosuppression via release of heme oxygenase-1. Heme oxygenase creates carbon monoxide, which suppresses the pro-apoptotic effects of TNFa on endothelial cells (Arnold et al., 2014).

Not every study suggests FAP has an immunosuppressive role. One study in non-small cell lung cancer used tissue microarray to identify correlations between CAF subtypes and immune markers. They demonstrated that in tumors with high CD3+/CD8+ T cell infiltration, high FAP expression was correlated with increased patient survival (Kilvaer et al., 2018). This study proposed a beneficial prognostic role of FAP+CAFs and warned that targeting FAP as a therapeutic approach should be done cautiously.

Tumor Suppression

With the amounting evidence to suggest FAP's role in tumor promotion, its potential as a tumor suppressor must be addressed. As previously discussed, FAP expression is specifically silenced in proliferating melanocytes undergoing malignant transformation. Melanocytes engineered to overexpress FAP or a catalytically inactive form of FAP regained contact inhibition, cell cycle arrest and increased susceptibility to stress-induced apoptosis. Furthermore, implantation of these FAP expressing melanocytes abrogated tumorgenicity in vivo (Ramirez-Montagut et al., 2004).

Signaling

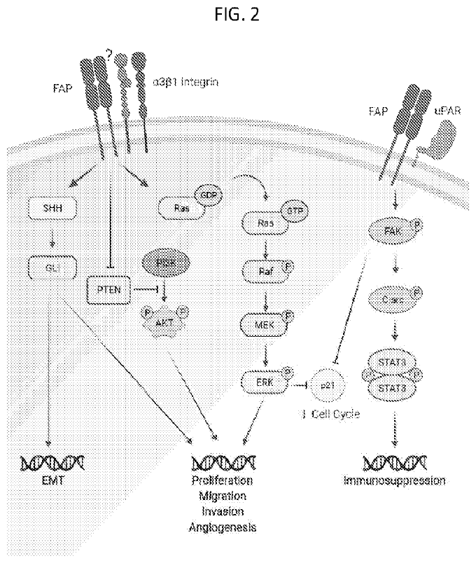

Several signaling pathways affected by FAP result in the phenotype witnessed in FAP expressing cells. Downstream signaling targets of FAP include PI3K/AKT, RAS/ERK, SHH/GLI, FAK and many others, as shown in FIG. 2. FIG. 2 shows potential signaling pathways affected by FAP that are responsible for the tumor promoting phenotypes associated with FAP expression.

PI3K/AKT: Cells engineered to overexpress FAP have increased proliferation and migration due to activation of the PI3K and the Sonic Hedgehog (SHH) pathways, which are intracellular signaling pathways required for cell cycle and differentiation, respectively. Exposure to inhibitors of PI3K and SHH abrogated the FAP induced phenotypic changes (Jia et al., 2014). In oral SCC cells, it has been reported that the knockdown of FAP resulted in suppressed proliferation, migration and invasion via inactivation the PTEN/PI3K/AKT and Ras-ERK signaling pathways (H. Wang et al., 2014).

FAK: Focal adhesion kinase (FAK), an intracellular tyrosine kinase recruited to the sites of integrin clustering or focal adhesions, functions as a major mediator of signal transduction by cell surface receptors, including integrins, growth factor and cytokine receptors. FAK partially regulates cell adhesion, migration, and invasion. Overexpression of FAP was associated with a decrease in phosphorylated FAK protein. One study suggested that FAP might form a complex with the FAK protein, and in doing so reduce its phosphorylation, which thus results in reduction of adhesion and motility ability (Jia et al., 2014). Furthermore, in FAP knockout mice, deletion of FAP increased p21 via ECM-mediated signaling through FAK and ERK (Santos et al., 2009). p21 is known to arrest the cell cycle. Therefore, FAP may inhibit the inhibitor, allowing for cell cycle progression and increased growth. In another study, FAP overexpression promoted proliferation in breast cancer cells in vitro. The addition of a FAK inhibitor reversed the proliferative ability of these cells, while inhibitors to PI3K, ERK and ROCK had no effect (Jia et al., 2014).

uPAR. FAP's association with uPAR has been implicated in both the cellular migration and immunosuppression phenotypes associated with FAP. In ovarian cancer cells, FAP complex with integrin a3B1 and the uPAR signaling complex mediated cellular migration via the small GTPase Rac1 pathway (Chung et al., 2014). In murine liver models, the expression of immunosuppressive cytokine CCL2 is mediated through a uPAR-dependent FAK-Src-STAT3 pathway, with STAT3 being the transcription factor responsible for Ccl2 expression. This paper validated these results in intrahepatic cholangiocarcinoma human specimens by tissue microarray, demonstrating that expression of FAP positively correlated with CCL2 and p-STAT3 levels (X. Yang et al., 2016).

SHH/GLI: In addition to SHI/GLI pathways' roles in promoting proliferation, invasion and migration as previously mentioned, FAP's effect on EMT may also be due to its activation of the SHH/GLI pathway. The expression of GLI1 was associated with changes in the expression of EMT markers E-cadherin and B-catenin in lung SCC specimens. Inhibition of the SHH/GLI pathway suppressed the migration of and upregulated E-cadherin in lung SCC cells. Conversely, stimulation of the SHH pathway increased migration and downregulated the expression of E-cadherin in the lung SCC cells (Yue et al., 2014). Since FAP overexpression activates the SHH a (Jia et al., 2017), FAP may be indirectly involved in the EMT process by regulating SHH. SHH has also been shown to promote the desmoplasia associated with pancreatic cancer (Bailey et al., 2008).

Therapeutic Targeting of FAP

While the function of FAP within malignancies remains poorly understood, there have been many efforts to exploit FAP biology clinically. Approaches that target FAP clinically include: inhibiting FAP's proteinase activity with small molecules or antibodies, using FAP proteinase activity to cleave oncologic drugs attached to peptides targeted to FAP, vaccination against FAP, and most recently, FAP CAR T cells.

Inhibition of Enzymatic Activity

Talabostat (Val-Boro-Pro, PT-100, BXCL-701) is one of the first small molecules designed to inhibit the dipeptidyl peptidase activity shared by DPPIV and FAP. Original pre-clinical work with the molecule was promising. Oral administration of talabostat slowed growth of syngeneic tumors derived from fibrosarcoma, lymphoma, melanoma, mastocytoma, rhabdomyosarcoma and bladder cancer cell lines in mice, in some instances causing complete regression and rejection of tumors (Adams et al., 2004; Walsh et al., 2013). Talabostat also enhanced the efficacy of oxaliplatin in murine models of colon carcinoma (M. Li et al., 2016). Talabostat's effects seemed immunologic in nature, as the anti-tumor effects were attenuated in immunodeficient mice. Talabostat enhanced cytotoxic lymphocyte anti-tumor effects, as CD8+ T cells from talabostat-treated mice had greater cytotoxic capabilities compared to untreated controls. This was further supported by data showing that talabostat enhanced the efficacy of tumor specific antibodies (Adams et al., 2004). Further studies suggested that talabostat enhanced dendritic cell trafficking, resulting in acceleration of T-cell priming. Interestingly, this study demonstrated that inhibition of extracellular FAP alone is insufficient to reduce tumor volume, thus suggesting that inhibition of intracellular dipeptidyl peptidases may be responsible (Walsh et al., 2013). To this point, one study suggested talabostat's mechanism of action was independent of its effects on FAP but rather depended on inhibition of DPP8/9, which induced pyroptosis in monocytes and macrophages that in turn activated the immune system (Okondo et al., 2017).

Despite the lack of consensus on talabostat's mechanism of action, it was further investigated in clinical trials. A phase I clinical trial of talabostat in relapsed or refractory pediatric solid tumors used maximal target inhibition to identify the appropriate dose of talabostat. At a dose of 600 ug/m2, there was serum DPPIV inhibition of 85% at 24 hours. No dose-limiting toxicities were observed, however the impact of talabostat on patient tumor growth could not be determined, since clinical development of talabostat was discontinued during the trial (Meany et al., 2010). A phase II clinical trial investigated talabostat as a single agent for advanced metastatic colorectal cancer. While the study identified no complete or partial responses, there were cases of prolonged stable disease in previously progressing tumors, suggesting possible anti-cancer activity. The patients enrolled in the study were heavily pre-treated and thus the lack of clinical response could have been attributed to the refractory patient population. An idea that is supported by the finding that FAP exerts greater biological effects at earlier stages in colorectal cancers (Henry et al., 2007). Other phase II trials investigated talabostat in combination with standard of care chemotherapeutics. A phase II trial assessing talabostat with cisplatin as second-line therapy in stage IV melanoma identified 8.1% of patients with partial response and 62.5% with stable disease. Of the patients who responded, the duration of response ranged from 62 to 287 days (Robert M Eager et al., 2009). A phase II trial of talabostat and docetaxel for advanced non-small cell lung cancer yielded two durable complete responses and three partial responses, for an overall response rate of 9.1% and a stable disease rate of 54% (R. M. Eager et al., 2009).

Talabostat has also been noted to have several side effects, most of which are related to cytokine release. The most common adverse events that could definitely be attributed to talabostat was edema. In the single agent trial there was one Grade 5 adverse event, a patient who died seven days after treatment due to acute renal failure due to cytokine storm. In the melanoma trial 56% of patients experienced grade 3 or 4 adverse events with 18% discontinuing talabostat due to the side effects. In the non-small cell lung cancer trial eight patients experienced adverse events resulting in death. However, none of these events were considered definitely or probably related to talabostat. The cytokine stimulation effects of talabostat may be clinically beneficial in cases of blood cell deficiencies. One study demonstrated that talabostat promoted growth of primitive hematopoietic progenitor cells by increasing G-CSF, IL-6, and IL-11 production from bone marrow stromal cells. Therefore, talabostat may be utilized to treat neutropenia or anemia (Jones et al., 2003).

Talabostat's nonspecific targeting of FAP complicates the ability to assess the effects of FAP inhibition on tumor growth. There has been an ongoing effort to develop an FAP-specific inhibitor to allow for better understanding of FAP biology as well as potentially improve FAP targeting clinically. Of note, Pieter Van der Veken's group has developed a compound, termed “compound 60” that selectively and completely inhibits FAP in murine models (Jansen et al., 2014). It should also be mentioned that DPPIV inhibitors are already an FDA approved class of drugs commonly utilized to treat Type II diabetes, because of their ability to enhance concentrations of incretins such as GLP-1.

Inhibition of FAP activity has also been attempted using antibodies. Early work on FAP-targeting monoclonal antibodies focused on clinical utility of the antibody originally used to identify FAP, F19. These studies did not investigate or expect improved clinical outcomes. Instead, they hoped that the elevated expression of FAP in both primary tumors and metastasis would mean that radioactively labeled F19 could improve imaging modalities in patients with hepatic metastases from colorectal carcinoma. In fact it did, with 131Iodine labeled F19 showing specific enrichment of the antibody in tumor areas and detection of metastasis. (Tanswell et al., 2001; Welt et al., 1994). These studies indicated potential diagnostic and therapeutic applications of FAP targeting antibodies. The first evidence that an anti-FAP antibody could suppress tumor growth came in 2002 from Louis Weiner's group. In this study, rabbits were immunized with recombinant murine FAP to obtain anti-FAP antisera. The anti-FAP antisera significantly attenuated tumor growth in colorectal carcinoma cell lines xenografted into nude mice (Cheng et al., 2002). Since then, specific anti-FAP antibodies and single-chain variable fragments (scFv) targeting FAP have been developed (A. Schmidt et al., 2001; J. Zhang et al., 2013).

ScFv are fusion proteins consisting of the variable regions of heavy and light chains of an immunoglobulin. These constructs have been further modulated to form bispecific antibodies capable of targeting both FAP and CD3 to target effector T cells to FAP expressing tumor tissue. In vitro studies demonstrated this FAP-CD3 bispecific antibody had enhanced cytotoxic activity against FAP expressing tumor cells (Hornig et al., 2012; Wüest et al., 2001). Then, sibrotuzumab, a humanized monoclonal anti-FAP antibody was produced. In a phase I dose escalation study in patients with advanced or metastatic FAP+ cancer, sibrotuzumab was proven safe as there was only one dose limiting toxicity during this trial. Unfortunately, there were no clinical responses and only 2/26 patients had stable disease (Scott et al., 2003). A phase II clinical trial of sibrotuzumab in metastatic colorectal cancer was suspended because of lack of clinical activity, although sibrotuzumab was well tolerated. (Hofheinz et al., 2003). Despite the disappointing results, the study of more efficient FAP antibodies continues. Radiolabeled human-mouse cross-reactive anti-FAP antibodies selectively accumulated in FAP expressing melanoma cell lines in vitro and in vivo. The uptake of radiolabeled antibody led to decreased tumor growth and improved survival murine models of melanoma (Fischer et al., 2012). While these studies show promise, more preclinical and clinical experiments are needed to explore the diagnostic and therapeutic effects FAP targeting molecules.

Prodrugs Utilizing FAP Proteinase Activity

Since FAP is overexpressed in the tumor microenvironment and is generally absent from other tissues in a healthy adult, some groups have focused efforts on utilizing FAP protease activity to selectively activate prodrugs at tumor sites to enhance drug efficacy and reduce toxicity. So far, these prodrugs have yet to make it to clinical trials but pre-clinical trials show promise. In a murine model of breast carcinoma, FAP overexpressing cancers showed equal sensitivity to epirubicin compared to compound that was an FAP substrate conjugated to epirubicin. Mice receiving the conjugated compound experienced less weight loss and less cardiotoxicity (J. Wang et al., 2017). A study of another anthracycline, doxorubicin, showed similar results with FAP substrate conjugated doxorubicin eliciting reduced toxicity to the heart, liver, kidney, spleen and peripheral white blood cells in both murine and canine models. The improved safety profile of this compound allowed for a two-fold increase in the dose of doxycycline administered in vivo (S. Huang et al., 2018). This technique was also applied to vascular disrupting agents. Administration of a vinblastine pro-drug conjugated to an FAP substrate markedly reduced tumor growth in tumors derived from HepG2, A549, HeLa, CNE-2 xenografts as well as ductal carcinoma and hepatocellular carcinoma patient-derived xenografts (Minfeng Chen et al., 2017).

FAP Vaccination

Vaccines targeting FAP provide another therapeutic strategy that takes advantage of the restricted distribution of FAP in tumor sites. Prophylactic vaccination with a DNA vaccine directed against FAP in mice inoculated with colon or breast carcinoma cells resulted in decreased tumor growth, suppressed pulmonary metastasis, increased chemotherapy uptake and increased survival in a CD8+ T cell dependent manner (Loeffler et al., 2006; Y. Wen et al., 2010). Another group engineered tumor cells to express murine FAP and then used the resulting whole cell vaccine with success. This FAP-expressing whole cell vaccine reduced tumor growth and improved survival in a CD8+ T cell dependent manner in both the prophylactic and post tumor inoculation settings (Meihua Chen et al., 2015). FAP vaccination has also been attempted with dendritic cell vaccines. A dendritic cell vaccine was developed to co-express FAP and tumor antigen tyrosine-related protein 2 had potent antitumor activity in murine models of melanoma (Gottschalk et al., 2013).

FAP CAR T Cells

Chimeric antigen receptor (CAR) T cells represent an exciting new class of immunotherapy strategies where cytotoxic T cells are engineered to recognize specific cancer antigens resulting in cancer cell elimination. CAR T cell therapy has already been approved by the FDA for some forms of leukemia and lymphoma (Ghobadi, 2018). The potential to use FAP CAR T cells to clear FAP expressing tumor cells was first demonstrated by Schuberth et al. In this study they demonstrated FAP CAR T cells successfully killer FAP expressing malignant pleural mesothelioma (MPM) lines and improved overall survival in murine models of MPM (Schuberth et al., 2013). However, expression of FAP by malignant cells is restricted to a few cancer types. Targeting FAP+ stromal cells with CAR Ts could greatly broaden FAP CAR T cell use. Further, given the pro-tumorigenic roles of FAP expressing CAFs, it is reasonable to hypothesize that using CAR T cells to selectively ablate FAP expressing cells could improve patient outcomes. Kakarla et al where the first to test if FAP CAR T cells could improve outcomes when used to deplete stomal cells. They showed that FAP CAR T cells effectively lyse FAP expressing target cell in vitro and improve mouse overall survival in murine models of lung adenocarcinoma (Kakarla et al., 2013).

Subsequent studies demonstrated FAP CAR T cells reduced tumor growth in murine models of lymphoma, mesothelioma and breast, colon and lung adenocarcinoma (L.-C. S. Wang et al., 2014). In this study they demonstrated FAP CAR T cells were ineffective in immunodeficient mice and showed FAP CAR T treatment enhanced endogenous tumoral T cell activity and infiltration. However, the clinical use of FAP CAR T cells should proceed with caution. One study showed that FAP CAR T cells failed to regulate tumor growth, and induced lethal bone toxicity and cachexia, potentially through the lysis of multipotent bone marrow stromal cells (Tran et al., 2013). The reason for the discrepancy in outcomes remains unclear, however it could be related to differences in FAP construct design and specificity, warranting further investigation into FAP CAR T cell optimization. Along these lines, one study demonstrated that the costimulatory domains expressed by FAP CAR T cells impacted their efficacy. In this study, the Δ-CD28 (which lacks the lck binding moiety) costimulatory domain resulted in superior tumor clearance when combined with anti-PD-1 than CD28 or 4-1BB costimulatory domains (Gulati et al., 2018). They also performed the first-in-human trial of FAP CAR T cells and demonstrated that a FAP CAR T cells therapy induced stable disease for one year in a patient with malignant pleural mesothelioma. Of note, this patient did not experience any treatment terminating toxicities. Lastly, FAP CAR T cells are might be efficacious in other diseases as well. Aghajanian et al demonstrated that FAP CAR T cells reduce cardiac fibrosis in murine models of cardiac fibrosis (Aghajanian et al., 2019).

Since the discovery of FAP, great strides have been made to better understand FAP biology. We now appreciate that its expression is not limited to activated fibroblasts, but includes endothelial, malignant epithelial, embryologic and immunologic tissues. Our understanding of its physiological role has expanded from simple collagen degradation to functions including activation of tumorigenic signaling cascades, angiogenesis, EMT and even immunosuppression. We also have learned that its physiologic functions may be independent of its peptidase activity and is instead dependent upon association with other molecules such as integrins and uPAR. Despite the apparent lack of FAP-targeting therapeutics clinical success, the striking occurrence of FAP in many pathologies continues to suggest it can provide some clinically targetable value.

Identification and Characterization of FAP in Immune Cells

Fibroblast activation protein-a (FAP), is predominantly expressed on cancer associated fibroblasts (CAFs) and minimally expressed on normal fibroblasts, normal or malignant epithelial cells or the stroma of benign epithelial tumors. From this original identification, FAP expression was believed to be exclusive to activated fibroblasts and has become one of the primary markers for CAF identification. As such, many laboratory techniques and FAP targeting drugs have been designed around this original set of observations.

Subsequent studies have challenged the concept that FAP expression is specific to fibroblasts. FAP expression was observed in some human malignant epithelial cell lines (Goodman et al., 2003; Iwasa et al., 2003; Mori et al., 2004, Okada et al., 2003; M. Shi et al., 2012), normal melanocytes (Huber et al., 2003) and human tumor associated macrophages (Tchou et al., 2013). While investigating the dynamics between immune cells and pancreatic cancer CAFs, we found that FAP expression is broadly expressed in human and murine leukocytes cell lines and further identify FAP expression in healthy donor derived NK cells but not human T cells, B cells or monocytes.

Materials and Methods

Cell Pellets, Lines and Culture

Primary human PSCs (ScienCell, cat #3830) were maintained on plastic and passaged every 1-3 days in stellate cell medium (ScienCell, cat #5301). For all experiments, PSC passage 7-11 was used. All human NK cell lines (NK92, NK92-GFP, NKL, YT, KHYG-1 NK92-CD16V) and murine NK cell lines (LNK) were kindly provided by Dr. Kerry S. Campbell (Fox Chase Cancer Center, Philadelphia, PA). The NK92-GFP expressed GFP due to transduction with pBMN-IRES-EGFP. All NK cell lines were cultured as previously described (Aldeghaither et al., 2019). Cell pellets were tested for FAP expression by western blot from the Jurkat, HuT 78, CCRF-CEM, Ramos, Namwala, IM-9, mono-mac 6, THP-1, U-937, Swiss3T3, RAW264.7, JAWSII, P815, BW5147.3, EL4 and A-20 cell lines obtained from the Georgetown Lombardi Comprehensive Cancer Center Tissue Culture Shared Resource.

Healthy Donor Derived Cells

Fresh healthy donor NK cells were purchased from AllCells with either CD56 positive selection or CD56 negative selection (Allcells, cat #PB012-P or PB012-N). T cells, B cells and monocytes were isolated from PBMCs (Allcells) using Mojosort magnetic cell separation system from Biolegend via CD3 positivity (Biolegend, cat #480133), CD19 positivity (Biolegend, cat #480105), CD14 positivity (Biolegend, cat #480093). PBMC purity was assessed using flow cytometry: CD3-APC (Biolegend, cat #300411), CD14-BV421 (Biolegend, cat #325627), CD45− FITC (BD Bioscience, cat #347463), CD56-PE (BD Bioscience, cat #555516), CD20-PE (BD Bioscience, cat #555623).

PSC-NK92 Coculture Assay

PSCs were plated one day prior to assay at 100,000 cells/well in a 6 well collagen coated plate. NK92 cells were added at 1:1 or 4:1 effector to target (E:T) ratios and cocultured for 3-4 hours. Each well contained 50% v/v NK and PSC media and 1% v/v IL-2. Following incubation, nonadherent cells were aspirated and collected. Adherent cells were washed 2× with PBS and then trypsinized with 0.05% trypsin. After detachment trypsin was quenched with equal volume PSC media and cells were collected, pelleted and washed 2× with PBS then resuspended in 600 uL of 1% BSA. Cells were immediately sent for nonsterile flow sorting of GFP+ from GFP− using the BD FACS Aria Ilu cell sorter in the Georgetown Lombardi Comprehensive Cancer Center Flow Cytometry and Cell Sorting Shared Resource (FCSR).

Microscopy

Cells were washed 2× with IX PBS, then fixed with 4% buffered formalin with 1% glutaraldehyde for 10 minutes at room temperature. Cells were washed 2× with dH2O. Hematoxylin stain diluted 50% with dH2O was added for 1 minute. Cells were washed 2-5× with dH2O. Plates were images using an Olympus 1X-71 brightfield/phase contrast microscope. Images were collected using an Olympus DP-70 camera and DP Manager v3, 1, 1, 208 acquisition software.

Annexin V Assay

One day prior to assay, PSCs were stained with Dil. PSCs were suspended at a density of 1×106 cells/mL in 1 mL of serum-free DMEM media (Thermofisher). 2 uL of DiI (Thermofisher) was added per every 1 mL of media. Cells were incubated with dye for 20 minutes at 37° C. and vortexed every 5 minutes. After incubation, cells were centrifuged for 5 minutes at 1000 rpm and then washed 2-3× with regular PSC media. Cells were then plated as described for the coculture assay. Following incubation period of 4 hours, all cells from a single well were collected and washed 2× with PBS. Samples were then processed by the FCSR using the Alexa Fluor 647 Annexin V and Sytox Blue staining (Biolegend). Flow data were analyzed using FloJo (v10.4.1), and GraphPad Prism 7 was used to conduct one-way ANOVA and subsequently Tukey's multiple comparison test.

FAP Activity Assay

One day prior to assay, 5,000 PSCs/well were added to 96 well flat clear bottom white polystyrene TC-treated microplates (Corning). The following day, PSC media was aspirated off and 50 uL of NK92 cells (lacking GFP) were added to each well containing PSCs at a 4:1 E:T ratio and incubated overnight at 37° C. 100 mM stock of dipeptidyl peptidase substrate (Acetyl-Aka-Gly-Pro-AFC) (Anaspec, CatAS-24126) was made by resuspending lyophilized substrate in DMSO. On the day of the assay, DMSO stock was then diluted 1:1000 in FAP activity assay buffer (50 mM Tris-BCl, 1 M NaCl, 1 mg/mL BSA, pH 7.5). A standard curve was generated using rFAP (R&D systems, 3715-SE-010). 50 uL of rFAP standard was added to wells in triplicate. 50 uL of substrate was added to each well and the plate was incubated for 5 minutes at 37° C. The plate was read on a PerkinElmer EnVision Multimode Plate Reader with 390400 nm excitation and 580-510 nm emission wavelengths. The final concentration of FAP per well was calculated using the standard curve. Data were compiled and assessed for significance using GraphPad Prism 7 for an unpaired, two-tailed t-test.

RNA Isolation and rt-qPCR

RNA was isolated using the PureLink RNA Mini Kit (Ambion). The RNA concentration was measured using NanoDrop 8000 (Thermo Fisher Scientific). cDNA was generated from 15-50 ng of RNA using the GoTaq 2-step RT-qPCR System (Promega). qPCR was performed with SYBR Green on a StepOnePlus real-time PCR system (Applied Biosystems). Gene expression was normalized to HPRT and analyzed using 1/DCt method with triplicates. Primers used were:

FAP
(F: ATGAGCTTCCTCGTCCAATTCA;
R: AGACCACCAGAGAGCATATTTTG)
HPRT:
(F: GATTAGCGATGATGAACCAGGTT;
R: CCTCCCATCTCCTTCATGACA)

Western Blot

Western blots were performed as previously described (Aldeghaither et al., 2019). Western blots were conducted using anti-FAP (ab207178, abcam) at concentrations of 1:1000 diluted in 5% milk in PBST. Secondary antibody was anti-rabbit IgG, HRP linked (Cell Signaling) used at 1:1000. Antibody was validated with additional anti-FAP antibodies (MBS303414, MyBiosource, and ab53066, abcam). GAPDH antibody (GAPDH (D16H11) XP Rabbit mAb, 5174S, Cell Signaling) was used at 1:10,000. The secondary antibody was anti-rabbit IgG, HRP linked (Cell Signaling) used at 1:5000. Chemiluminescent substrate (Pierce) was used for visualization. Densitometry was measured using ImageJ (v1.48).

Immunohistochemistry

PSCs were scraped and pelleted at 1000 rpm for 5 minutes. Media was aspirated off and pellets were fixed using 20 mL of 10% neutral buffered formalin. Pellets were sent to VitroVivo Biotech, LLC for histogel embedding, sectioning and staining with anti-FAP antibody ab207178 (abcam) at a dilution of 1:200. The slides were imaged using the Olympus BX61 DSU Fluorescent scope and images were acquired using CellSens Software.

mRNA Expression Using (CLE

Salmon version 0.4.2 transcript quantified RNA-sequencing data (Patro et al., 2017) from CCLE with reference genome GRCh37.74 were obtained from the Translational Genomics Research Institute (TGen): Quantified Cancer Cell Line Encyclopedia (CCLE) RNA-seq Data. Gene level counts were subset to cell lines of interest and variance stabilized with the rlog function from the R/Bioconductor package DESeq2 version 1.20.0. ENSEMBL ids were mapped to gene symbols with the org.Hs.eg.db package version 3.8.0. FAP expression was obtained from variance stabilized expression and exported to GraphPad Prism 5 was used for data presentation.

Single-Cell RNA-Seq

Processed gene expression data and corresponding cell type estimation from head and neck squamous cell carcinoma patients was obtained from GEO (GSE103322) (Puram et al., 2017). Imputation was performed using MAGIC version 0.1.0 (Python) prior to analysis (van Dijk et al., 2018). Batch effect correction was performed using the function ComBat from R/Bioconductor package sva version 3.26.0 (Leek et al., 2012) considering each patient as a batch to isolate differences between cells from distinct HNSCC cell types in each tissue.

Cell Surface Biotinylation

Cell surface biotinylation in NK92-CD16v, NKL, YT and KHYG-1 cells were performed with the Pierce Cell Surface Protein Isolation kit (Thermo Scientific) according to the manufacturer's protocol. In brief, 4×108 cells were pelleted and washed with cold PBS then incubated with EZ-LINK Sulfo-NHS-SS-biotin for 30 min at 4° C. followed by the addition of a quenching solution. Another 1×106 cells were collected and saved for total cell western blotting. Cells were lysed with lysis buffer (500 μL) containing the cOmplete protease inhibitor cocktail (Roche, 11697498001). The biotinylated surface proteins were isolated with NeutrAvidin agarose gel, eluted in 250 uL of Pierce Lane Marking non-reducing sample buffer (Pierce, 39001) diluted 1:5 in ultrapure water supplemented with DTT to a final concentration of 50 mM. Lysates were subjected to Western blotting with the anti-CTLA-4 antibody described above.

Flow Cytometry

Cell pellets were collected via centrifugation at 1000 rpm for 5 mins Cells were washed 2× with 1×PBS and resuspending in 100 uL staining buffer (1% BSA in PBS). 1 uL of human Fc block was added (BD Pharmingen, cat #564219) and incubated at 4° C. for 45 minutes. 4 uL of 0.25 mg/mL sheep anti-human FAP antibody (R&D systems, cat #AF3715) or 0.5 uL of 2 mg/mL sheep IgG control (R&D systems, cat #5-001-A) was added and cells incubated at 4° C. for 30 minutes, vortexing half way through. Cells were washed 2× with staining buffer then resuspended in 100 uL staining buffer. 2 uL of PE-conjugated donkey anti-sheep secondary was added (R&D systems, cat #F0126) and incubated at 4° C. for 30 minutes in the dark, vortexing half way through. Cells were washed 2× with staining buffer, resuspended in staining buffer. Samples were run in the Georgetown Lombardi Comprehensive Cancer Center Flow Cytometry Cell Sorting Shared Resource using BD LSRFortessa. Analyses were performed using FlowJo (v10.4.1).

Results

In Vitro Coculturing Model for Investigating Relationship Between Human Natural Killer Cells and Human Pancreatic Stellate Cells

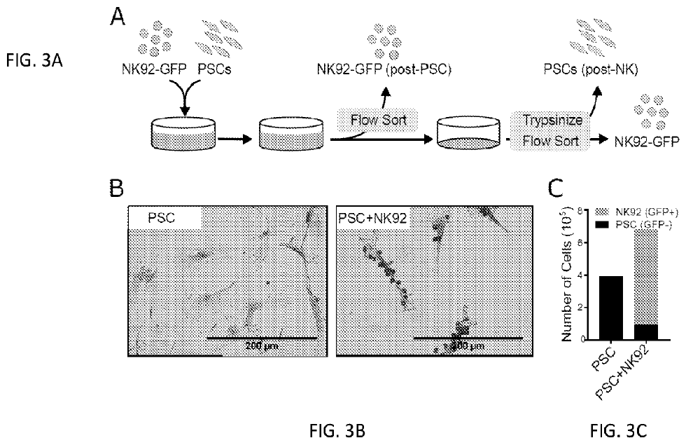

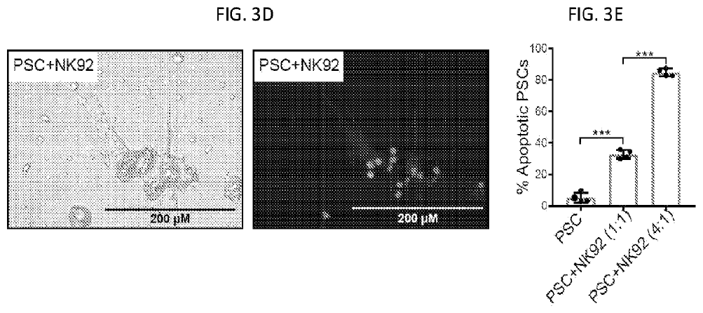

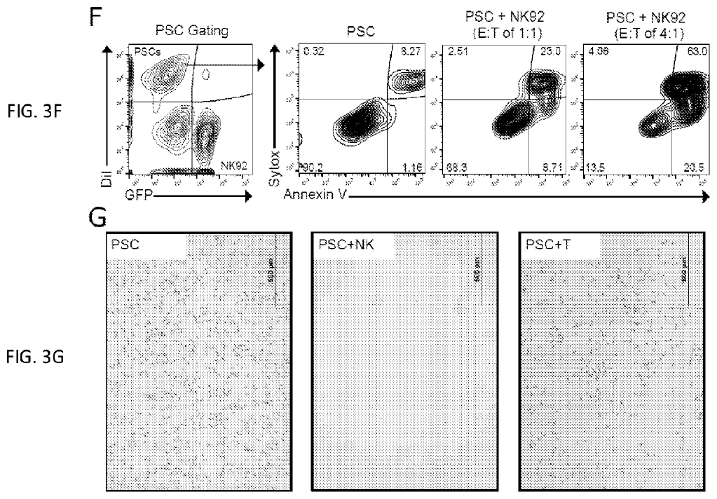

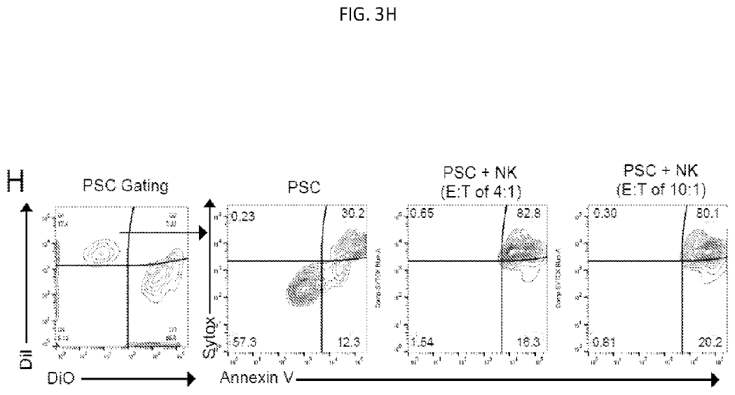

To investigate the relationship between NK cells and stromal components of pancreatic cancer we established a novel in vitro coculture system that utilizes adherent primary human PSCs and a GFP expressing human NK cell line (NK92-CD16v-GFP), which grows in suspension. Following coculture, media containing NK cells is collected. After the plate is washed with PBS, the remaining cells, which include PSCs adherent to the plate and NK cells bound to the PSCs, are trypsinized to detach them from the plate and each other. These cells are then FACS sorted into GFP+ and GFP− populations to separate the GFP+ NK92 cells from the GFP− PSCs (FIG. 3A). This system allows for the separate interrogation of PSCs and NK cells following coculture.

NK92 Cells Bind to and Kill Human PSCs

We began investigating the relationship between NK cells and PSCs using in vitro coculture. First, we assessed the physical appearance of NK-PSC coculture. After three days of coculture, NK92 cells adhered to PSCs (FIG. 3B). Adherence was quantified by flow cytometry to determine the number of PSCs and NK92 cells present following coculture. PSCs cultured alone contained a negligible number of GFP positive cells, as expected. Following coculture, NK92 cells accounted for approximately 86% of all adherent cells (FIG. 3C). To confirm the dense spheres observed in FIG. 3B were NK cells and not morphological alterations of PSCs we imaged the cocultured using GFP fluorescent microscopy and confirmed that coculture resulted in adherent, GFP+ cells (FIG. 3D). To determine if the NK92 cells were killing the PSCs we performed an Annexin V flow cytometry assay to detect live, necrotic, early apoptotic, and late apoptotic cells. PSCs were pre-stained with DiI. The DiI+/GFP− gate was used to specifically assess apoptosis in the PSC population. In the PSC population pre-NK exposure, approximately 8% of cells were apoptotic. The percentage of apoptotic PSCs increased significantly after a 4-hour coculture with NK92 cells. When equal numbers of NK92 and PSCs were co-cultured, e.g., an effector-to-target ratio (E:T) of 1:1, approximately 35% of PSCs were apoptotic. When the E:T ratio was increased to 4:1, approximately 90% of PSCs were apoptotic (FIGS. 3E and 3F). We confirmed that primary healthy donor NK cells, but not T cells, lyse PSCs (FIGS. 3G and 3H). These data demonstrate confirm previous reports that NK cells can lyse PSCs (Van Audenaerde et al., 2017).

NK Cell Lysis of PSCs is Dependent, in Part, on NKG2D

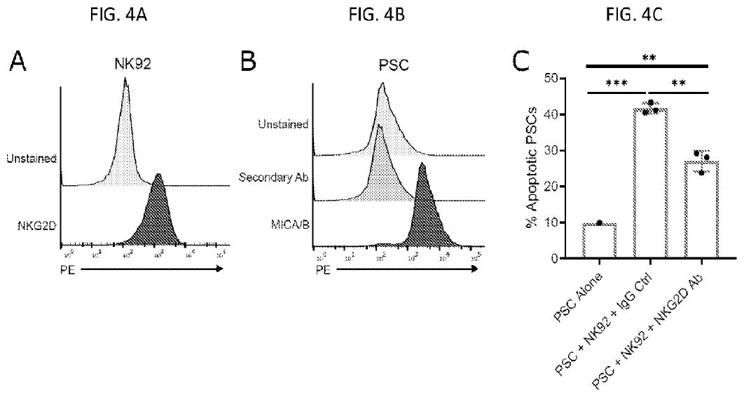

Van Audenaerde et al. were the first to demonstrate human NK cells could lyse PSCs in vitro. However, an earlier study investigating the relationship between murine NK cells and hepatic stellate cells in liver fibrosis reported that murine NK cells lysed hepatic stellate cells via NK cell activating receptors TRAIL and NKG2D (Radaeva et al., 2006). We confirmed that NK92 cells express NKG2D (FIG. 4A) and the primary PSCs in our system express NKG2D ligands MICA/B (FIG. 4B). The addition of an NKG2D blocking antibody reduced NK92 lysis of PSCs by approximately 25%, however the NKG2D blocking antibody did not completely ablate NK92 lysis of PSCs (FIG. 4C). These findings suggest that NKG2D is involved in NK lysis of PSCs, but not the only receptor mediating target cell lysis.

PSCs Reduce FAP Expression Following Co-Culture with NK92 Cells

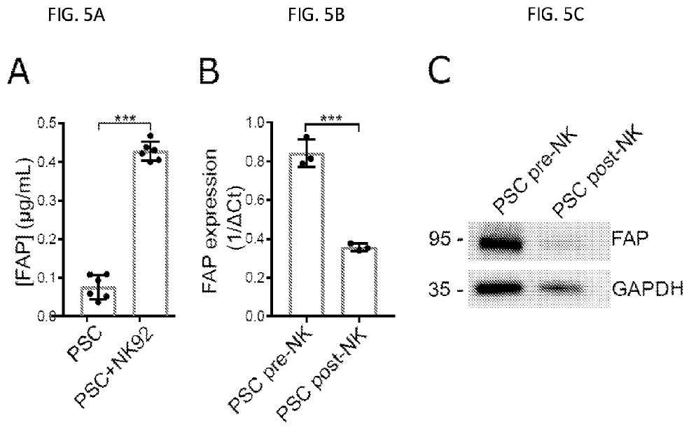

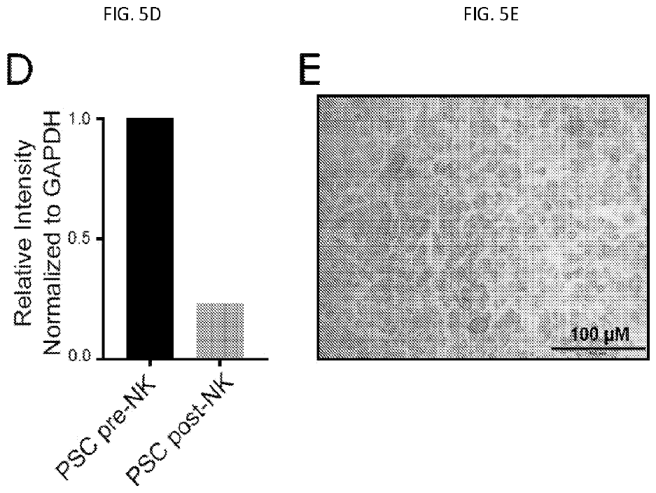

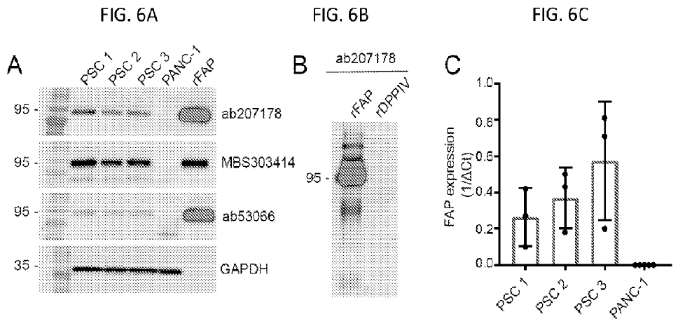

While PSCs are known to overexpress FAP in PDAC lesions (Ohlund et al., 2017), the exact mechanism by which FAP is induced in PSCs is unknown. Since FAP is overexpressed in many diseases characterized by inflammation, such as arthritis (Scott et al., 2003), Crohn's disease (Rovedatti et al., 2011) and atherosclerosis (Brokopp et al., 2011), we hypothesized that FAP could be induced in PSCs in response to immune attack. To test this hypothesis, we cocultured PSCs with NK92 cells and assessed FAP enzymatic activity using a fluorescently labelled peptide substrate. Upon coculture, there was a more than 4-fold increase in FAP activity when compared to the PSCs alone (FIG. 5A). However, this assay did not discriminate which cell type in the coculture was responsible for increased FAP activity. Accordingly, we employed the FACS sorting technique to separately examine the cell populations. Rt-qPCR analysis surprisingly demonstrated that FAP mRNA levels were decreased in the PSCs post-NK92 cell exposure compared to PSCs pre-NK92 exposure (FIG. 5B). These findings were confirmed by western blot (FIGS. 5C and 5D). Western blot antibody specificity was confirmed using a positive control cell line (PSC) and recombinant protein (rFAP), as well as a negative control cell line (PANC-1, previously demonstrated to be FAP negative (Tyulkina et al., 2016)) and recombinant protein (rDPPIV) with multiple anti-FAP antibodies (FIGS. 6A and 6B). The control cell lines' FAP expression was confirmed using rt-qPCR (FIG. 6C). We considered two potential causes for decreased FAP expression in PSCs following NK cell exposure. The first is that in response to immune attack, PSCs downregulate FAP. The second is that PSCs with high FAP expression level are preferentially killed by NK cells. Immunohistochemistry analysis of cultured PSCs demonstrates heterogeneous FAP expression (FIG. 5E). If PSCs with elevated FAP expression are preferentially targeted by NK cells the remaining PSC population following NK cell exposure would consist of PSCs with low levels of FAP expression and reflect less FAP by bulk rt-qPCR and western blot analysis. Future studies will determine how FAP expression is reduced in PSCs following immune attack.

NK92 Cells Express FAP

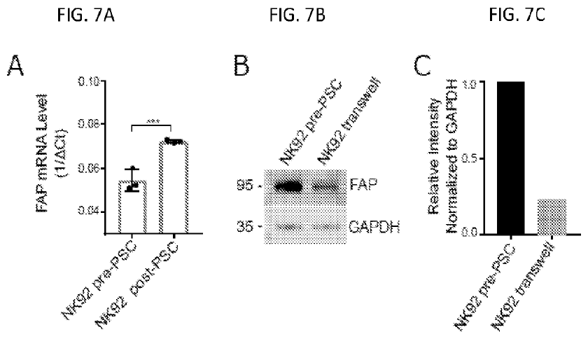

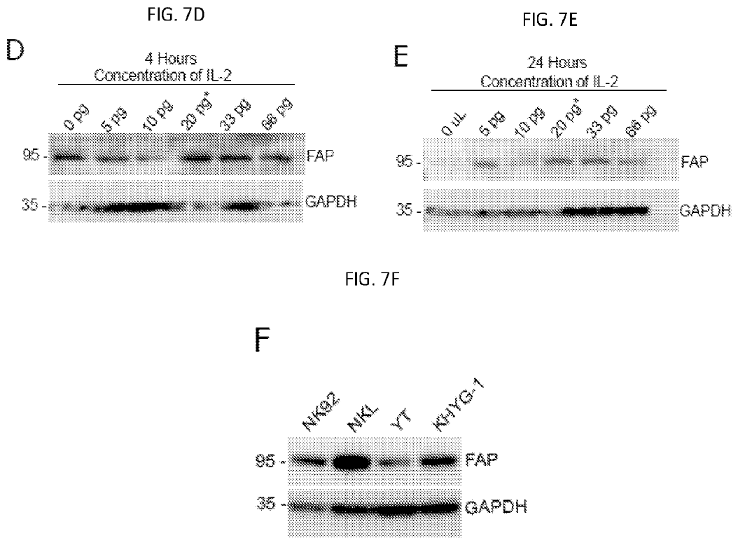

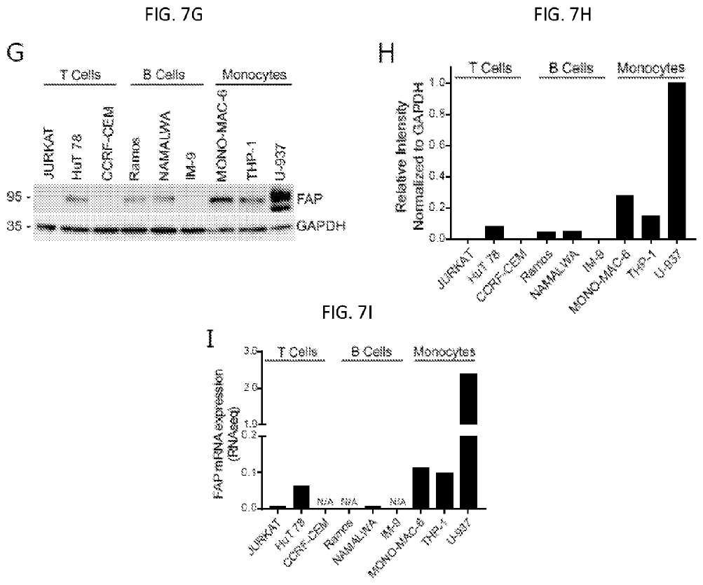

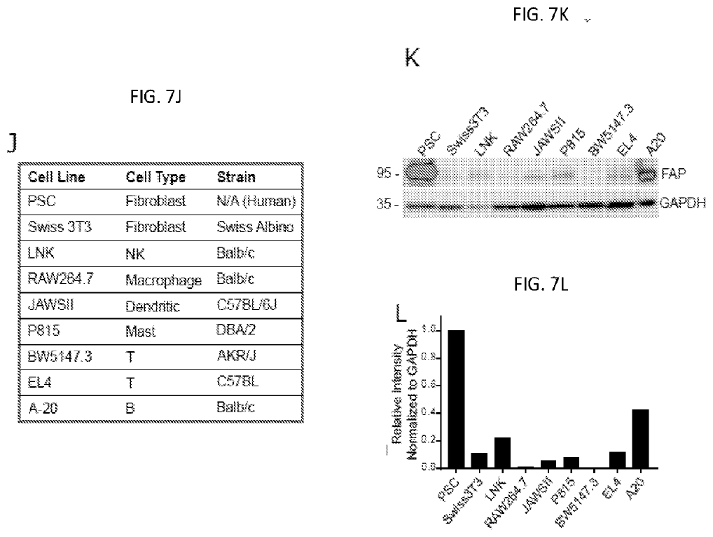

Since RT-qPCR and western blot showed that the increased FAP activity seen in FIG. 2.3A could not be attributed to PSCs, we investigated FAP expression levels in NK92 cells. Rt-qPCR analysis of NK92 cells pre- and post-PSCs exposure showed not only FAP expression in NK92 cells prior to PSC exposure, but also increased FAP expression following exposure (FIG. 7A). Interestingly, this increase in FAP expression was only seen following direct contact with PSCs. Coculture of NK92 cells with PSCs in a transwell system that allowed for communication but not contact did not result in increased FAP expression (FIGS. 7B and 7C). These results suggest that FAP expression in NK cells is induced upon direct contact or by a factor released after direct contact with a target cell. IL-2 was investigated as a potential regulator of FAP expression due to its upregulation and release following NK cell activation. The NK cell line, NKL, was exposed to increasing concentrations of IL-2 and FAP protein levels were assessed at 4 and 24 hours after IL-2 exposure (FIGS. 7D and 7E). IL-2 exposure did not induce FAP expression. Future studies are required to identify factors that modulate FAP expression during or after contact with PSCs.

Additional Human NK Cell Lines and Primary NK Cells Express FAP

To determine if FAP expression was an NK92 cell line-specific phenomenon we assessed FAP expression in three additional human NK cell lines (NKL, YT and KHYG-1). All three additional NK cell lines expressed FAP as determined by western blot (FIG. 7F). This is the first report of FAP expression by human natural killer cells.

FAP is Heterogeneously Expressed in Other Human and Murine Immune Cell Lines

To determine if FAP is expressed by other human immune cell populations, western blot was performed on three T-cell (Jurkat, HuT 78, CCRF-CEM), B-cell (Ramos, Namwala, IM-9) and monocyte (mono-mac-6, THP-1, U-937) cell lines (FIGS. 7G and 7H). FAP was heterogeneously expressed in both the T-cell and B-cell populations. One of the three T-cell lines tested expressed FAP. Two of the three B-cell lines tested expressed FAP. All three of the monocyte lines tested expressed FAP and expression levels were higher than those seen in T and B cells. Protein expression was validated with FAP mRNA expression levels using the Broad Cell Line Encyclopedia (CCLE) (Barretina et al., 2012) (FIG. 7I). The RNA-seq analysis showed that FAP mRNA levels correlate with FAP protein expression in the cell lines tested. To determine if FAP expression by leukocytes was exclusive to human leukocytes we assessed various murine immune cell lines that reflect multiple immune cell types derived from multiple strains (listed in FIG. 7J) for FAP expression by western blot (FIGS. 7K and 7L). Just as with human leukocytes, FAP expression is heterogeneous in murine leukocyte cell lines.

FAP is Expressed by Healthy Human Donor NK Cells

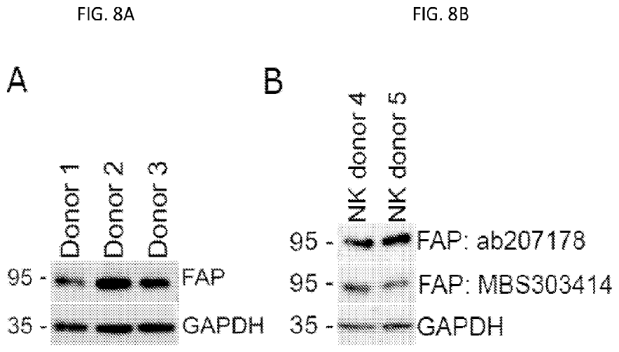

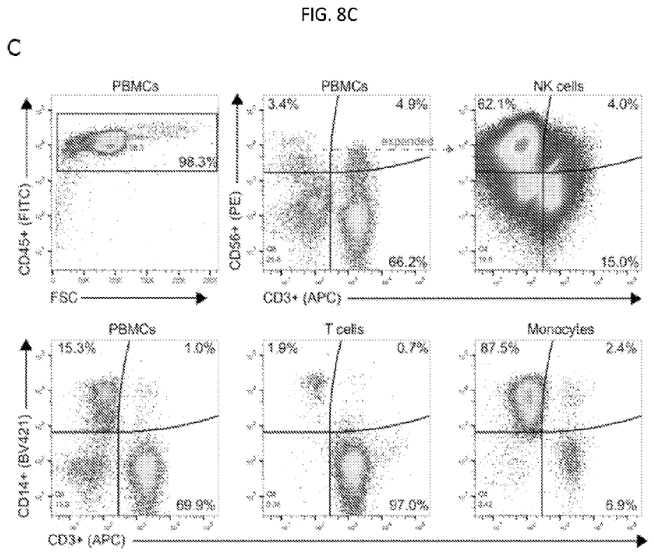

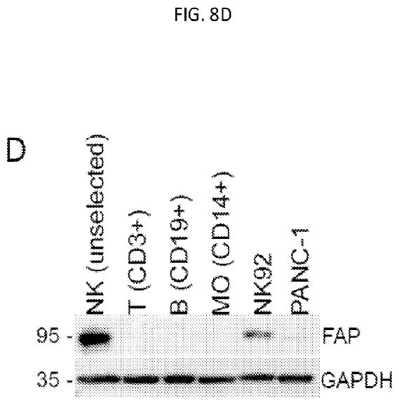

To determine if the expression of FAP could be attributed to malignant transformation, we investigated FAP expression healthy human donor immune cells by western blot. Circulating NK cells from three separate healthy human donors selected by CD56 positivity were purchased. All three donors had detectable FAP protein levels as determine by western blot (FIG. 8A). We further confirmed two addition human donor-derived NK cells expressed FAP using two anti-FAP antibodies (FIG. 8B). Given that immune cell lines from immune cell lineages other than NK cells had detectable FAP expression by western blot (FIG. 7G) we hypothesized that like NK cells, other immune cell types isolated from the blood of healthy human donors would also have detectable levels of FAP. PBMCs were purchased and CD3+ T cells, CD14+ monocytes, and CD19+ B cells were positively selected using magnetic bead purification. Following isolation, the immune cell populations were assessed for purity using flow cytometry. CD3+ T cells were 97% pure, the CD14+ monocytes were 89% pure and the remaining, unpurified population was 33.9% CD56+/CD3− NK cells (FIG. 8C). Surprisingly, only NK cells had detectable levels of FAP protein expression by western blot (FIG. 8D).

Leukocytes Express Less FAP than Cancer Associated Fibroblasts

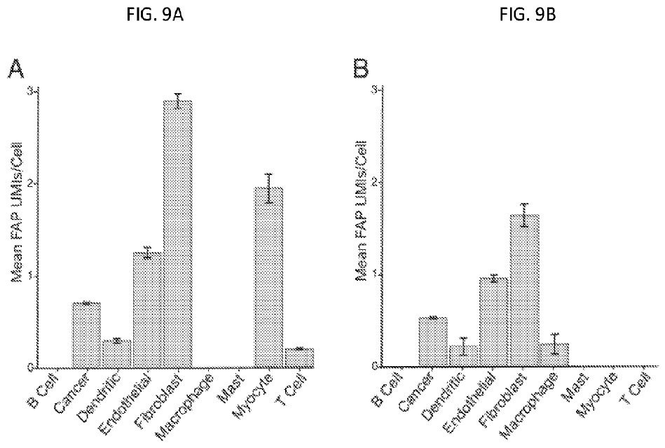

We considered several potential explanations for why FAP expression by immune cells has not been reported previously. The first hypothesis is that the relative overexpression of FAP in cancer associated fibroblasts (CAFs) resulted in a diminished ability to detect FAP expression in the immune cell populations. To determine the relative expression of FAP in different tumoral cell populations we assessed single-cell RNAseq data obtained from 16 primary tumors and 5 lymph node metastases of head and neck squamous cell carcinoma patients (FIG. 9A) (Puram et al., 2017). As expected, FAP expression in CAFs was nearly 10-fold greater than that of dendritic cells, which is the immune cell population with the greatest amount of FAP expression in this dataset. Interestingly, FAP expression in all cell types, excluding macrophages, appeared lower in the lymph node metastasis in comparison to the primary tumor (FIG. 9B). This would not have been predicted given that FAP has been hypothesized to enhance tumor migration and invasion (H. Chen et al., 2009b; Ghersi et al., 2006; Yuan Zhang et al., 2011).

FAP is Expressed on NK Cell Surface Yet Undetected by Flow Cytometry

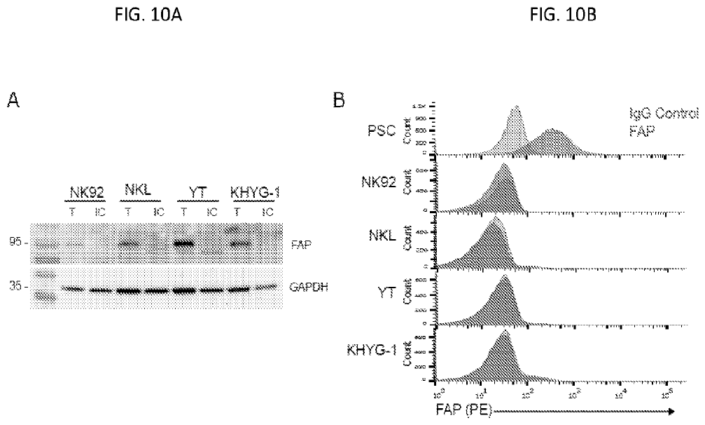

We also speculated that FAP has gone undetected in leukocytes because the epitope identified by most IHC or flow antibodies is hidden or altered when FAP is expressed by leukocytes as compared with fibroblasts. It has been well documented that FAP can bind to various cell surface molecules such as uPAR and integrins (Chung et al., 2014; H.-O. Lee et al., 2011; Mueller et al., 1999; W. Yang et al., 2013). Accordingly, it is possible that when FAP is expressed by leukocytes, it is bound to cell surface molecules that mask the epitope. We confirmed FAP was expressed on the surface of NK cell lines using biotinylation isolation of surface expressed proteins (FIG. 10A). However, FAP was undetected on the surface of NK cells by flow cytometry (FIG. 10B). Subsequently, KHYG-1 cells were investigated for intracellular expression of FAP by flow cytometry and was not detected (data not shown). This could explain why FAP has gone largely undetected in IHC or flow cytometry analysis of leukocytes to date but can be readily identified by gene expression and under the reducing conditions of western blot shown.

Discussion

Previous studies have identified FAP+CD45+ cells in the murine tumor environment. Kraman et al. identified FAP+CD45+ cells in LL2 Lewis lung carcinoma murine models and suggested these cells resembled CD11b+/class 11+/Col 1+/a-SMA+ fibrocytes (Kraman et al., 2010). Using the same murine model this group also identified a population of FAP+F4/80hi/CCR2+/CD206+M2 macrophages that contributed to tumoral immunosuppression via release of heme oxygenase-1 (Arnold et al., 2014). Additionally, Tchou et al. identified FAP+CD45+ cells in human breast cancer specimens using IHC (Tchou et al., 2013). Subsequent flow cytometry for macrophage markers CD14, CD11b, HLA-DR, and CD114 demonstrated these cells were tumor-associated macrophages. However, this study did not examine other leukocyte markers and as such the possibility that additional CD45+ cell types express FAP could not be excluded. This is the first report of FAP expression by human NK cells. FAP is currently considered a primary marker, along with aSMA, of CAFs because of its presumably restricted expression pattern. In studies that assess CAFs, they are often first identified by expression of CAF markers including FAP, aSMA or PDGFa and then subsequently phenotyped (Omland et al., 2017). Alternatively, bulk FAP expression analysis has been used as a surrogate for fibroblast activation and CAF presence (Allaoui et al., 2016). Based on our findings, results from experiments that utilized such methods may benefit from further interrogation to determine if the samples contain FAP+ leukocytes.

FAP expression by human natural killer cells implies that FAP may have additional and as yet uncharacterized biological functions. Traditionally, FAP has been believed to promote tumor growth by enhancing tumor cell invasion and migration through its extracellular matrix remodeling protease activity and/or intracellular effects that promote cell growth and migration. Recent studies have described a role for FAP in anti-cancer immunity by demonstrating that FAP expressing cells release immunosuppressive cytokines (Arnold et al., 2014: Feig et al., 2013; Kraman et al., 2010). Our findings implicate FAP more directly in immunity and suggest that FAP may play a more central role in immune cell function and regulation. Lastly, leukocyte expression of FAP has potential clinical implications. For example, an anti-FAP antibody fused to IL-2 has been developed (Klein et al., 2013). The proposed mechanism of action of this compound is that the anti-FAP antibody targets the IL-2 to the tumor and as such activates only tumoral T and NK cells, thereby enhancing IL-2 efficacy and reducing cytotoxicity. However, our data suggest that this compound could have an alternative mechanism of action by targeting the IL-2 to natural killer cells directly. In summary, we demonstrate that FAP is robustly and constitutively expressed by healthy donor NK cells and thus should be considered in future studies that investigate FAP biology, FAP-targeting therapeutics, and FAP based laboratory methods.

Murine Models to Elucidate FAP Function in NK Cells

To determine if and how FAP expression by NK cells alters NK cell anti-tumor activity, we tested two different FAP inhibitors (BXCL701 and Cpd60) in syngeneic murine models of pancreatic ductal adenocarcinoma (PDAC). We selected PDAC as our model because PDAC is an exceptionally deadly cancer with an overall 5-year survival rate of less than 8% (Siegel et al., 2018). Clinically, PDAC is characterized by poor response to chemotherapy, radiotherapy and immunotherapy (Orth et al., 2019). Pathologically, PDAC is characterized by extensive desmoplastic stroma, with up to 90% of the tumor volume being stroma (Neesse et al., 2011). The majority of PDAC specimens overexpress FAP but it is absent in non-PDAC controls (M. Shi et al., 2012). PDAC patients with high tumor FAP expression have a shorter overall survival (Kawase et al., 2015). Because of the elevated levels of FAP expression, correlations between high FAP and worse clinical outcomes, and dire need for therapeutic advancements, we chose PDAC to study the effects of an FAP inhibitor. BXCL701 (i.e. Talabostat, PT-100, Val-boro-Pro) is a non-specific FAP inhibitor that also inhibits DDP4, DPP8 and DPP9 (Adams et al., 2004). BXCL701 is currently being tested in pre-clinical and clinical trials to treat a variety of malignancies, either alone or in combination with chemotherapeutics or immunotherapies. Cpd60 is a specific FAP inhibitor (Jansen et al., 2014) that is less well studied than BXCL701. We performed initial exploratory studies using BXCL701, then assessed if the changes induced by BXCL701 were due to FAP inhibition or not by repeating these experiments with an FAP specific inhibitor (i.e. Cpd60).

Materials and Methods

TCGA Analysis Cell Lines

mT3-2D murine pancreatic cancer cell lines were gifts from David Tuveson, Cold Spring Harbor Laboratory, Laurel Hollow, NY (Boj et al., 2015). The mT3-2D-GFP/luc cell line was a gift from Chunling Yi, Georgetown University Lombardi Comprehensive Cancer Center, Washington, DC. Briefly, pHAGE PGK-GFP-IRES-LUC-W (addgene, cat #46793) was transfected into 293T cells to generate the virus. The virus was infected into mT3-2D cells and GFP positive cells were FACS-sorted. All these cell lines are syngeneic in C57BL/6 mice. All cell lines were grown in standard conditions and maintained in Dulbecco's Modified Eagle Medium (DMEM) (Fisher Scientific, cat #SH30022LS) supplemented with heat-inactivated fetal bovine serum (HI-FBS) to a final concentration of 10% and 2 mM L-glutamine. All cell lines were tested and determined to be free of Mycoplasma and other rodent pathogens.

Drug Preparation

20-28 mg of BXCL701 was diluted in 0.1 N HCl then to obtain a final concentration of 20 mg/mL. BXC1701 was then diluted 1:100 in dH2O. Cpd60 was first dissolved in DMSO, then PEG 200 then water for final concentration of 6.6 mg/mL in 0.8% DMSO, 30% PEG 200.

Animal Studies

For subcutaneous experiments, 1×105 mT3-2D cells were injected subcutaneously into the right flank of C57BL/6J wild-type mice. Mice were given either 30 ug BXCL701 daily by oral gavage in 100 uL PBS or 200ug of anti-PD1 (clone: RMP1-14, BioXcell) twice per week by intraperitoneal (i.p.) injection or both. Treatment started when tumors reached about 50-100 mm3 and continued for 3-4 weeks as designated. All tumors were measured twice-weekly using calipers. Mice were euthanized at end of treatment or when tumors reached 1-2 cm3 or when mice showed signs of pain or distress, via C02 inhalation. Volume was calculated using (length×width 2)/2.

For the orthotopic experiment, 5×104 mT3-2D-GFP/Luc were orthotopically implanted into the pancreas using survival surgery. After one week, treatments were initiated with either 30 ug Cpd60 daily by oral gavage in 100 uL PBS or 200ug of anti-PD1 (clone: RMP1-14, BioXcell) twice per week by intraperitoneal (i.p.) injection or both. The tumor growth was monitored weekly using the IVIS imaging system (Xenogen Corp, Alameda, CA). 30 mg/kg luciferin (Nanolight Technology) was administered to mice i.p. to image tumors. Mice were euthanized using C02 inhalation when orthotopic tumors reached after three weeks of treatment, when tumors reached 1×1010 radiance, or when mice showed signs of pain or distress, whichever came first. After euthanizing the mice, tumors were excised, and tissue samples were collected for downstream analysis. All mice used in the study were 6-8 weeks of age and purchased from The Jackson Laboratory (Bar Harbor, ME). All studies involving animals were reviewed and approved by the Georgetown University Institutional Animal Care and Use Committee (GU IACUC).

For immune cell depletion studies, depletion started 1 day prior to treatment initiation before tumor cell inoculation. CD8+ T cells, NK1.1+NK cells or both were depleted using 200 μg of 200 μg anti-CD8 antibody (BioXCell, cat #BE0061) or 200 μg anti-NK1.1 antibody (BioXCell, cat #BE0036) twice weekly for the first two weeks then once weekly until the end of the experiment. After euthanizing, murine splenocytes were collected to evaluate efficacy of depletion using PE anti-NK1.1 (Biolegend, cat #108707) and PE/Cy7 anti-CD8 (eBioscience, cat #25-0083).

Dipeptidyl Peptidase Activity Assay

100 mM stock of dipeptidyl peptidase substrate (Acetyl-Aka-Gly-Pro-AFC) (Anaspec, cat #AS-24126) was made by resuspending lyophilized substrate in DMSO. On the day of the assay, substrate stock was then diluted 1:1000 in activity assay buffer (50 mM Tris, 1 M NaCl, 1 mg/mL BSA, pH 7.5). A standard curve was generated using rFAP (R&D systems, cat #3715-SE-010) or rDPP4 (R&D systems, cat #9168-SE) and 50 uL of recombinant protein plus 50 uL of substrate was added to each well of a 96 Well Flat Clear Bottom White Polystyrene TC-Treated Microplates (Corning, cat #3903). The plate was incubated for 30 minutes at 37° C. then read on a PerkinElmer EnVision Multimode Plate Reader with 390-400 nm excitation and 580-510 nm emission wavelengths. To test tumor lysates, 50-100 mg of snap frozen tumors were homogenized in 100-300 uL activity assay buffer using a Kimble Biomasher II closed system tissue grinder (DWK Life Sciences, cat #749625-0020). Protein concentration was determined using Bio-rad protein concentration DC Protein Assay Kit II (Bio-rad, cat #5000112). All samples were diluted to equal protein concentrations using activity assay buffer then added at 50 uL plus 50 uL substrate per well. Final concentration of FAP per well was calculated using the standard curve. Data were compiled and assessed for significance using GraphPad Prism 9 for an unpaired, two-tailed t-test.

Endopeptidae Activity Assay

FAP specific activity assay was based off of work done by Brainbridge et al. (Bainbridge et al., 2017). A fluorescent peptide substrate was synthesized by Anaspec (HiLyteFluor488-Val-D-Ala-Ser-Gln-Gly-Lys-QXL520). A 65.66 mM stock was made by adding 100 uL of DMSO. The day of the assay the substrate was diluted to 13 uM in assay buffer (50 mM HEPES, 150 mM NaCl, 1 mM EDTA, 0.1 mg/mL BSA, pH 7.5). 50/50 v/v of substrate/sample was added to each well. For serum samples, 25 uL of serum was combined with 25 uL of activity assay buffer.

Immunohistochemistry (IHC)

For solid tumors, tissues were fixed in 10% formalin overnight at room temperature, and then stored in 70% ethanol until paraffin embedding. Samples were sent to the Georgetown University Histopathology and Tissue Shared Resource for embedding, sectioning and staining. ImageJ (v1.48) and FIJI (v2.0.0-rc-69/1.52n) were used for the analysis. Antibodies used were: anti-mouse CD8 (Cell Signaling, cat #98941), anti-mouse CD4 (Cell Signaling, cat #25229), anti-mouse CD335/NKp46 (R&D Systems, cat #AF2225), anti-mouse CXCR3 (Bioss, cat #BS2209R).

Cytokine Panel

Tumors were harvested and homogenized in phosphate-buffered saline (PBS)+0.5% Tween-20 with protease inhibitors (Roche; Penzberg, Bavaria, Germany). Homogenates were centrifuged and the supernatant was immediately stored at −80° C. Samples were shipped to Eve Technologies for processing.

Flow Cytometry

Spleen tissue was gently ground between frosted glass microscope slides. Tissue was passed 10× through a 1000 μL pipette tip. Red cells were removed by incubating the splenocytes for 3 minutes with 3 ml eBioscience 1×RBC Lysis Buffer (Invitrogen, ThermoFisher, #00-4333-57). Cells were pelleted by centrifugation, and then recovered in 10 ml RPMI media with 10% HI-FBS, and filtered using a 70 μm cell strainer. Tumors were chopped into small pieces that were then transferred into gentleMACS tubes (MACS Miltenyi Biotec), containing 10 ml of DMEM media and 1 mg/ml collagenase D (Sigma-Aldrich, COLLD-RO Roche, #11088866001). The tubes were placed on a gentleMACS Dissociator (MACS Miltenyi Biotec, #130-095-937) using the program 37_m_TDK2. After incubation, cells were filtered using 70 μm cell strainer and recovered by centrifugation. Cells were stained for live/dead with either LIVE/DEAD Fixable Violet Dead Cell Stain Kit, for 405 nm excitation (ThermoFisher, cat #L34963) or Zombie NIR (Biolegend, cat #423105) then stained with a cocktail of surface mAbs: BV711 anti-CD45 (Biolegend, cat #103147), PE anti-NK1.1 (Biolegend, cat #108707) and PE/Cy7 anti-CD8 (eBioscience, cat #25-0083), APC anti-CD4 (eBioscience, cat #14-0042-81). After 30 minutes of staining, cells were washed and samples were run on FACS Symphony cytometer (BD Biosciences). Flowjo v10 was used for the analysis, cells were manually gated on size and granularity. Dead cells and doublets were excluded, and CD45+ cells were selected.

Results

DPPs are Overexpressed in Pancreatic Cancer Compared to Normal Pancreas

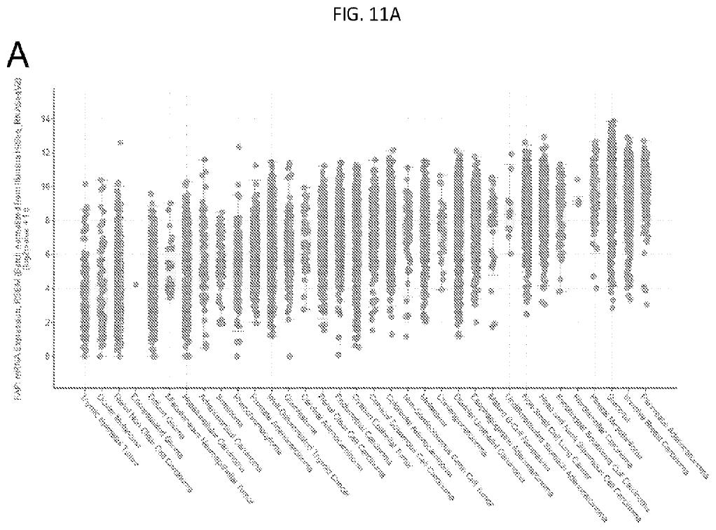

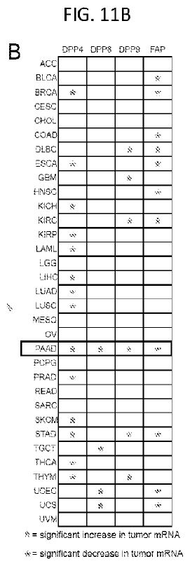

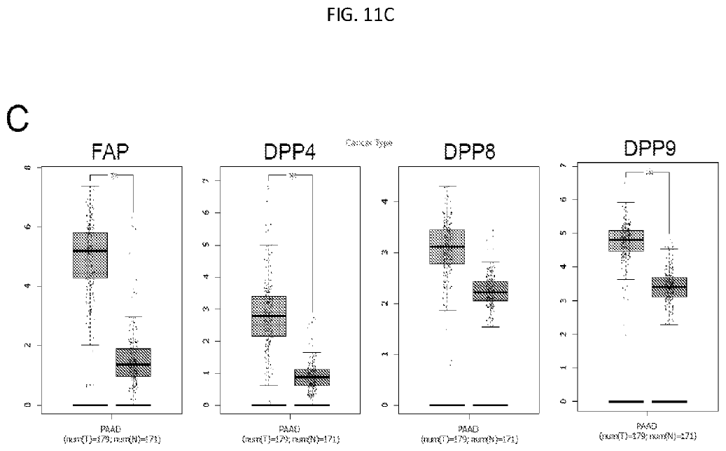

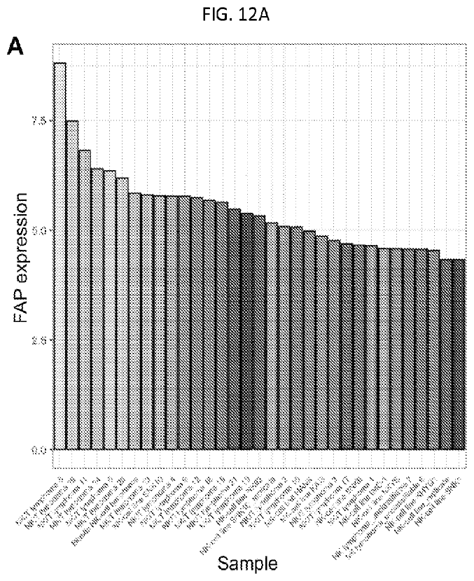

To investigate the potential importance of dipeptidyl peptidases (DPPs) in pancreatic cancer biology we used transcriptional data from TCGA. We first assessed FAP expression across different solid malignancies and found that pancreatic cancer tumors had the highest levels of FAP expression (log RSEM units) (FIG. 11A, gray box). We next investigated the relative expression of DPP4, FAP, DPP8 and DPP9 in malignant tumors versus health tissue by comparing tumor expression in TCGA data with matched normal tissue from healthy donors in the GTex dataset. No tumors had significant overexpression of DPP8. There were only two tumor types that had significantly increased expression of three DPPs (DPP4, FAP and DPP9): pancreatic adenocarcinoma (PAAD) and stomach adenocarcinoma (STAD) (FIG. 11B). The difference in FAP, DPP4, DPP8 and DPP9 expression in PAAD tumors (n=179) versus normal pancreas (n=171) is shown in FIG. 11C.

Zebrafish and 3D Models to Elucidate FAP Function in NK Cells

Natural killer (NK) cells are innate lymphoid cells that influence many physiologic and pathologic conditions-especially viral infections and cancers-through their effector and regulatory cell functions (Vivier et al., 2008). NK cells are canonically known to recognize and kill aberrant cells, such as virus infected or malignant cells, using a complex detection system comprised of multiple inhibitory and activating receptors. Beyond their roles as effector cells, NK cells also regulate the functions of other cells types, such as dendritic cells, T cells, B cells and endothelial cells, through the release of immunomodulating cytokines (Belyakova et al., 2019; Deniz et al., 2008; F. D. Shi et al., 2000; Shimoda et al., 2015; Walzer et al., 2005).

Because of their central role in the immune system and disease etiologies, efforts to manipulate NK cell activity have long been sought and developed to improve patient outcomes across many medical fields. In cancer, patients with high tumoral NK cell content and activation have improved survival (Cursons et al., 2019; B. Li et al., 2020) and response to immunotherapy (Barry et al., 2018; Davis-Marcisak et al., 2020; H. Lee et al., 2019). Because of this, NK cells are emerging as major targets to promote cancer immunotherapy (Souza-Fonseca-Guimaraes et al., 2019). Current NK-focused immunotherapy approaches include autologous or allogenic NK cell transfer (Sakamoto et al., 2015), CAR NK cells (E. Liu et al., 2020), NK immune checkpoint inhibitors (Fayette et al., 2018), bi- or tri-specific killer engagers (BiKEs and TriKES) (Sarhan et al., 2018), and cytokine super-agonists (Felices et al., 2017). An impediment to all these therapies is inadequate NK cell honing to and/or infiltration into solid tumors.

Strategies that increase NK cell infiltration into tumors represent plausible ways to enhance NK cell-related antitumor immunotherapies. Such work has focused almost entirely on modulating NK chemokine receptors and chemoattractants (Kremer et al., 2017; Wennerberg et al., 2014). However, lymphocyte migration depends on more than just chemotaxis. For NK cells to successfully infiltrate any tissue, including solid tumors, they must traverse diverse, complex microenvironments including extravasation from blood vessels and navigation through dense extracellular matrices (Kameritsch & Renkawitz, 2020). Beyond the chemokine/chemoattractant system, little is known about the mechanisms NK cells employ to physically migrate through these tissues.

Here it is described for the first time that human NK cells express fibroblast activation protein (FAP). FAP is a transmembrane serine protease primarily expressed on activated fibroblasts during wound healing or pathological conditions such as fibrosis, arthritis, and cancer (Fitzgerald & Weiner, 2020). Since FAP is overexpressed in diseased tissue yet mostly absent from healthy tissue (Fitzgerald & Weiner, 2020), it is a promising therapeutic target in conditions like cardiac fibrosis (Aghajanian et al., 2019) and cancer (Busek et al., 2018). FAP is primarily known for its extracellular matrix remodeling capabilities due to its collagenase activity. After identifying FAP expression by human NK cells, we used computational approaches to elucidate FAP's function in NK cells. We validated these computational findings using 2D assays. We then explored the impact of FAP inhibition on NK cell migratory properties such as extravasation using zebrafish models and tumor infiltration and lysis using 3D coculture systems. We found that FAP regulates human NK cell migration, extravasation, and infiltration into matrix-containing tumors which ultimately affects tumor cell lysis. These findings demonstrate the necessity of proteolytic migration in NK cell function, suggest novel mechanisms of action of FAP targeting drugs, and provide an entirely new way to regulate NK cell activity.

Materials and Methods

Donor NK Cell Expansion

For donor NK cell lysis of PANC-1 clusters, primary donor NK cells were purchased from Allcells then expanded using irradiated K562-4-1BBL-mbIL-21 (names “CSTX002”) cells kindly provided by Dr. Dean Lee according to his protocol (Somanchi et al., 2010).

Computational Analyses

NK lymphoma and cell line gene expression was downloaded from GEO (GEO accession GSE19067) (27) using R version 3.8.2 and read using affy in Bioconductor (57). Non-NK cell samples were excluded from analysis. Heatmap was created using ComplexHeatMap version 2.1.1 (58). Correlation analysis was performed using limma in Bioconductor (59). Gene set enrichment analysis was performed using GO enrichment (60).

2D Migration Studies

2D migration studies were done as previously reported. In brief, EL08.1D2 stromal cells were grown to a confluent monolayer on Lab-Tek chamber slides pre-coated with 0.1% gelatin (Stemcell Technologies). 10 uM of Cpd60 in RPMI media was added to the chamber 15 min before imaging. Cells were imaged in 96-well ImageLock plates (Essen Bioscience) on the IncuCyte ZOOM Live-Cell Analysis System (Essen Bioscience) at 37° C. every 2 min in the phase-contrast mode (10× objective). Tracking of live cells was done using the manual tracking feature in Volocity or FIJI. Tracks were plotted using the Chemotaxis plugin of FUI. Cells that were in the field of imaging for fewer than two frames were discarded, as were cells which were non-adherent or floating. EL08.1D2 cells were used as de facto fiducial markers to ensure that neither they or the microscope stage was drifting and causing apparent NK cell movement.

Length and displacement measurements were derived directly from tracked cells and graphed using GraphPad software. Velocity data was obtained by dividing the total track length by the time of imaging.

Zebrafish Studies

The zebrafish embryos were anesthetized with 0.0003016% tricaine (Pentair Aquatic Eco-Systems, Sigma-Aldrich, St. Louis, MO, USA) in the Georgetown-Lombardi Animal Shared Resource and positioned within our zebrafish stereotax on a proprietary microinjection plate. NK92-GFP cells were injected into the pericardium using an air driven Picospritzer Ha microinjector (General Valve/Parker Hannifin) under a stereoscope. After transplantation, embryos were allowed to recover for 1 hour at 33° C. Confocal imaging was performed on an Olympus IX-71 inverted microscope with a color CCD camera in the Georgetown-Lombardi Microscopy Shared Resource. Fluorescent imaging was performed on Keyence BZ-X. Images were taken at 10× across multiple z-stacks. Z-stack images were compressed using full focus and haze reduction in Keyence BZ-X software. NK extravasation quantification was performed by counting the number of GFP cells outside red vasculature. NK extravasation quantification was performed blinded to the treatment conditions. Graphs of resulting data and statistical analysis was generated using Graphpad Prism 9.

Spheroid Studies

PSC or PANC-1 spheroids were generated by plating 10,000 cells in a 0.1% agarose coated U-bottomed 96-well plate. PSC+PANC-1 spheroids were generated by plating 5,000 cells of each cell type. Aggregation was promoted by centrifuging the cells at 1000 rpm for 5 minutes. Cells incubated overnight at 37° C. The next day, 1,000 NK92-GFP cells were added per well and incubated for 4 hours at 37° C. Spheroids were imaged using the Olympus IX-71 Inverted Epifluorescent Microscope at 5×. Images were analyzed in FIJI. All images underwent identical contrast enhancement and background reduction. Then a line was drawn from spheroid edge to spheroid center and GFP intensity along that line was measured. Graphs of resulting data and statistical analysis were generated in Graphpad Prism 9.

3D Cluster Studies

3D clusters were generated, embedded and stained as previously described. In brief, clusters were generated by plating 1,000 cells per well into 96-well Nunclon Sphera low adhesion plates (Thermo Scientific, cat #174925) and incubated overnight at 37° C. The following day, 6 clusters were embedded into an ECM containing 2,000 NK cells and plated into one well of a Nunc Lab-Tek II 8-well chamber slide (ThermoScientific, cat #154534PK). The ECM mixture consisted of 20% growth factor reduced Matrigel (Corning, 10-12 mg/ml stock concentration, #354230) and 80% rat tail collagen type I at 3 mg/mL (gibco, A1048301). Cells were either imaged for the following 24 hours every 30 minutes using the Zeiss LSM800 scanning confocal microscope or allowed to incubate overnight at 37° C. After 24 hours, cells in matrix were fixed with 5.4% formalin for 1 hour, permeabilized with 0.5% Triton-X and blocked using goat serum. For invasion assays, NK-92-GFP cells were stained with anti-GFP (ThermoFisher, cat #A-11122). For the cell lysis assays clusters were stained using anti-cleaved caspase 3 (Cell Signaling, cat #9661). Hoechst 33342, phalloidin, and secondary antibodies labeled with Alexa Fluor 488 nm, 546 nm, 647 nm, or 680 nm (Invitrogen) were used.

Results

In Human NK Cells, FAP Gene Expression Correlates with Extracellular Matrix and Migration Regulating Genes

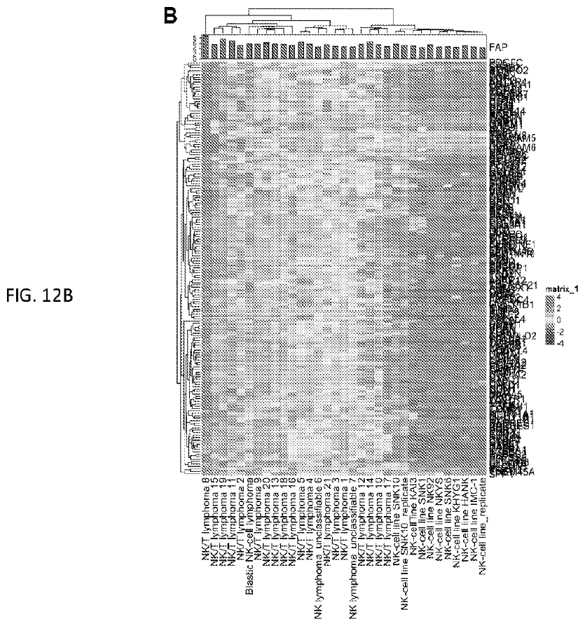

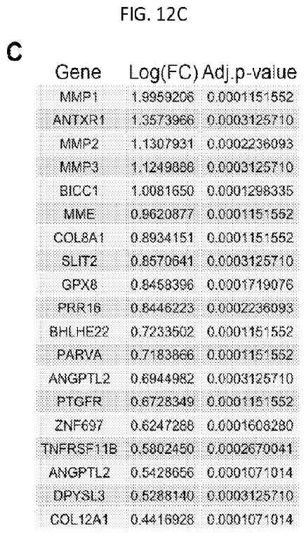

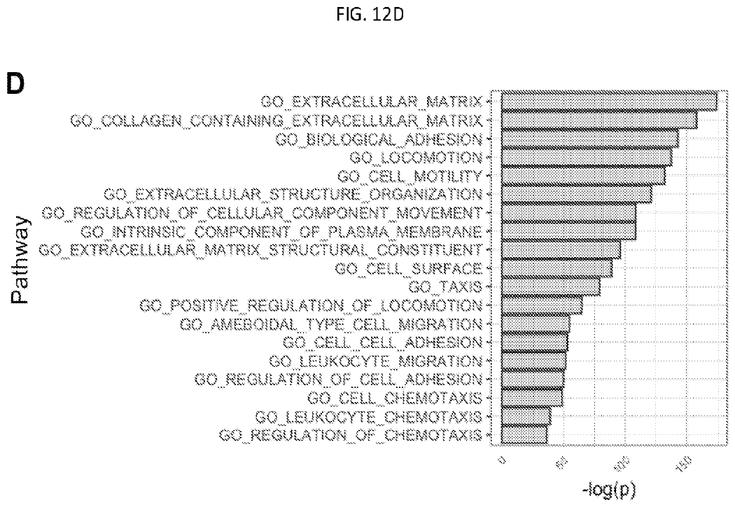

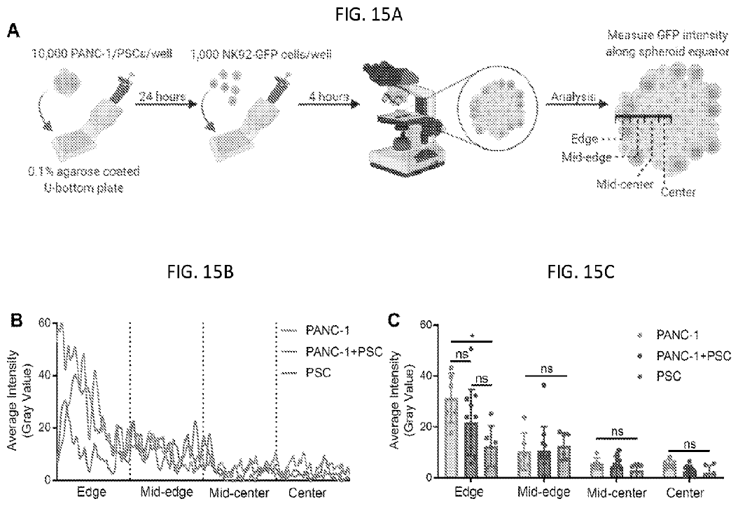

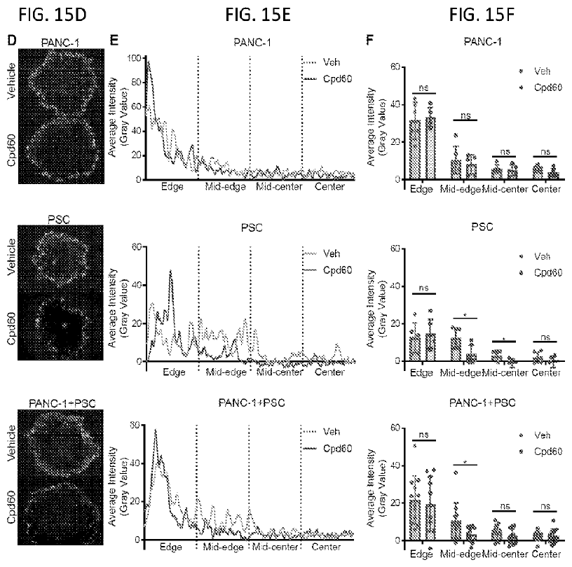

To determine FAP's function in human natural killer cells we employed computational approaches. In 2011, Iqbal et al. performed a gene expression array on multiple NK cell lymphoma samples and NK cell lines (Iqbal et al., 2011). Using these data, we assessed FAP expression in 22 NK cell lymphomas and 11 NK cell lines (FIG. 12A) and performed a correlation analysis to assess the genes that were most positively and negatively correlated with FAP expression (FIG. 12B). The top 19 genes that were most positively correlated with FAP expression are shown in FIG. 15C. We then performed GO enrichment analysis of these genes and determined that the pathways most positively correlated with FAP expression were related to extracellular matrix remodeling and cellular migration (FIG. 12D). This is consistent with the current understanding of FAP function, which is to cleave extracellular matrix components such as collagen and enhance cellular migration/invasion (Fitzgerald & Weiner, 2020). It is also interesting that matrix metalloproteases (MMPs) were among the top 19 genes positively correlated with FAP expression. MMPs regulate rat, mouse and human NK cell migration into collagen or Matrigel in vitro (Goda et al., 2006; Goldfarb Nannmark et al., 2000; Kitson et al., 1998). These data suggest that FAP may also regulate NK cell migration.

FAP Inhibition Reduces Primary NK Cell Migration

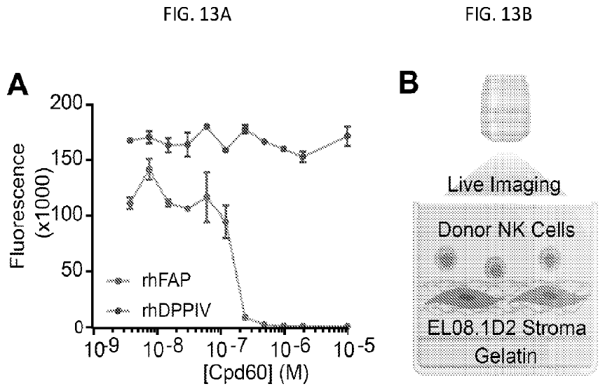

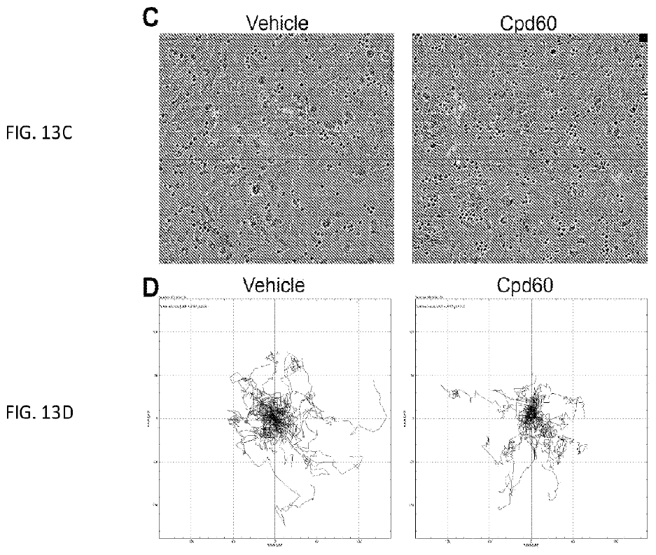

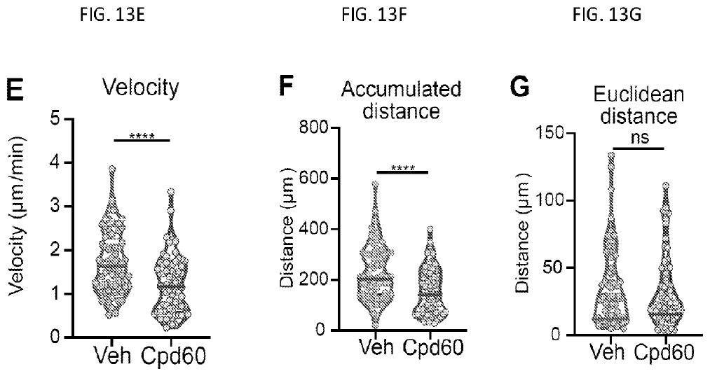

Based on the computational analysis, we hypothesized that FAP was expressed by human NK cells to enhance their migration. To test this hypothesis, we compared primary NK cell migration ex vivo in the presence and absence of an FAP-specific inhibitor (Cpd60) (Jansen et al., 2014) that inhibited FAP but not FAP's most closely related protein, DPPIV or other members of the prolyl oligopeptidase family S9 (FIG. 13A). Cpd60 had no effect on NK cell viability. We then cocultured primary NK cells with EL08.1D2 cells, which have previously been shown to support spontaneous NK cell migration and produce extracellular matrix, and live imaged them for 24 h capturing photos every 2 minutes (FIG. 13B). From these time-lapse videos we were able to track NK cell migratory paths (FIGS. 13C and 13D). These experiments were repeated with NK cells from three different donors, with similar results. We found that FAP inhibition with Cpd60 significantly reduced NK cell velocity (FIG. 13E) and the accumulated distance traveled by NK cells (FIG. 13F) but had no significant impact on the Euclidian distance—the straight-line distance between the starting point and end point—traveled by NK cells (FIG. 13G).

FAP Inhibition Reduces NK Cell Extravasation In Vivo

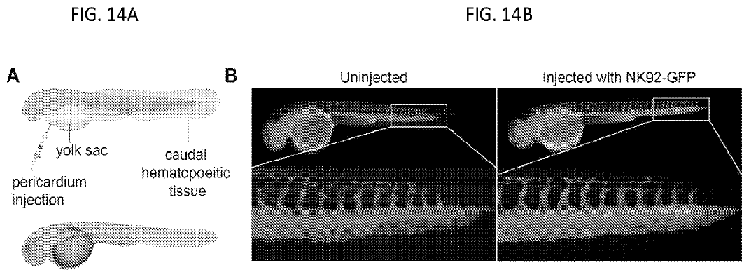

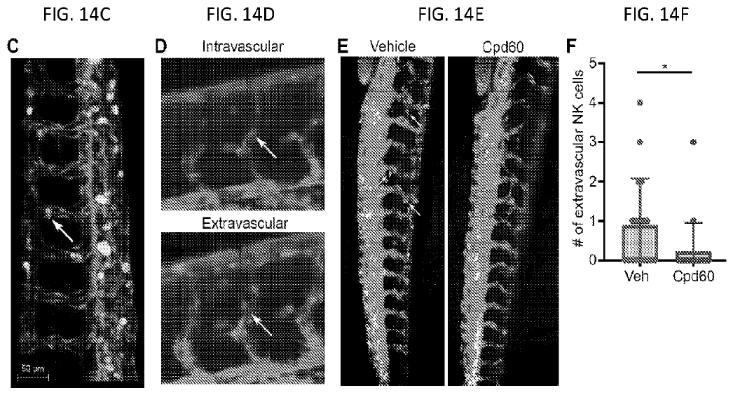

We next set out to determine if FAP altered NK cell migratory behaviors in vivo. Since we could not detect FAP expression in murine NK cells, we opted to use zebrafish—a novel in vivo model that allows us to monitor human NK cell migratory behaviors in real-time. We injected NK92-GFP cells into the pericardium of Tg(kdrl:mCherry-CAAX)y171 zebrafish embryos that express endothelial membrane-targeted mCherry (FIG. 14A). Immediately after pericardial injection, NK cells rapidly migrated to the caudal hematopoietic tissue (FIG. 14B) before disseminating throughout the rest of the zebrafish vasculature. Using confocal live-imaging, which captured images approximately every 3 minutes, we captured an NK cell crawling along the inside of the blood vessel, searching for an appropriately sized pore just prior to extravasation (FIG. 14C). After confirming that human NK cells could migrate throughout and extravasate from zebrafish vasculature, we tested the effects of FAP inhibition on NK cell extravasation. Since fluorescent microscopy is more amenable to imaging multiple fish simultaneously, we used fluorescent microscopy to quantify the effects of the FAP inhibitor Cpd60 on NK cell extravasation. We confirmed that the fluorescent microscope was capable of detecting NK cell extravasation (FIG. 14D), and then imaged 20 fish injected with NK92-GFP cells, 10 of which were bathed in 10 uM of Cpd60, and 10 fish that were bathed in vehicle. We found that FAP inhibition significantly reduced NK cell extravasation from the blood vessels (FIG. 14E and FIG. 14F).

FAP Inhibition Reduces NK Cell Infiltration into Matrix Containing PDAC Tumor Spheroids

NK cells regulate tumor growth and viability, yet the mechanisms NK cells employ to migrate through dense tumor-related extracellular matrix is unknown. To determine if FAP activity affects NK cell infiltration into tumors we used tumor spheroid models of PDAC generated from the PDAC cell line PANC-1 and primary pancreatic stellate cells (PSCs, PSCs comprise the majority of cancer-associated-fibroblasts in PDAC tumors). Homogeneous PANC-1 tumor spheroids have minimal extracellular matrix but PSC and PSC+PANC-1 heterogeneous tumor spheroids contain rich stroma that contains extracellular components such as collagen and fibronectin (H. J. Hwang et al., 2019; Ware et al., 2016). We generated homogenous PANC-1 or PSC spheroid and heterogeneous PANC-1+PSC spheroids by plating 10,000 cells in a 0.1% agarose coated U-bottom plate and allowed the spheroids to form over 24 hours. After 24 hours, 1,000 NK92-GFP cells were added to the spheroids and 4 hours later the spheroid-NK cell cocultures were imaged using fluorescent microscopy. To assess the extent of NK cell infiltration into tumor spheroids, we measured GFP intensity along an equatorial line drawn in FUI. We then divided this line into quarters, which we termed “edge”, “mid-edge”, “mid-center” and “center”, then averaged the intensity along those quarters (FIG. 15A).

As expected, NK cells infiltrated into PANC-1 spheroids more readily than they infiltrated PSC spheroids (FIG. 15B and FIG. 15C). This is likely because PANC-1 spheroids do not contain extracellular matrix like PSC spheroids; PANC-1 spheroids thus lack the physical matrix barrier that impedes NK cell infiltration. We next assessed the impact of FAP inhibition on NK cell infiltration into homogenous PANC-1 spheroids, homogenous PSC spheroids, and heterogeneous PANC-1+PSC spheroids (FIG. 15D, FIG. 15E and FIG. 15F). We found that 10 uM Cpd60 significantly reduced NK cell content in the mid-edge and mid-center regions of the PSC spheroids, and significantly reduced NK cell content in the mid-edge region of the PSC+PANC-1 spheroids, yet had no effect on NK cell content in any region of the PANC-1 spheroids. These results suggest that FAP regulates NK cell migration through tumors, but only in the presence of extracellular matrix.

FAP Inhibition Reduces NK Cell Infiltration into and Lysis of PANC-1 Cell Clusters Embedded in Matrix

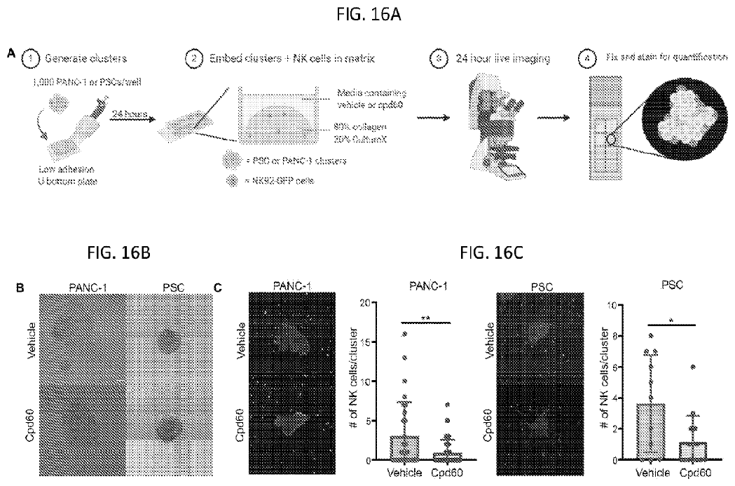

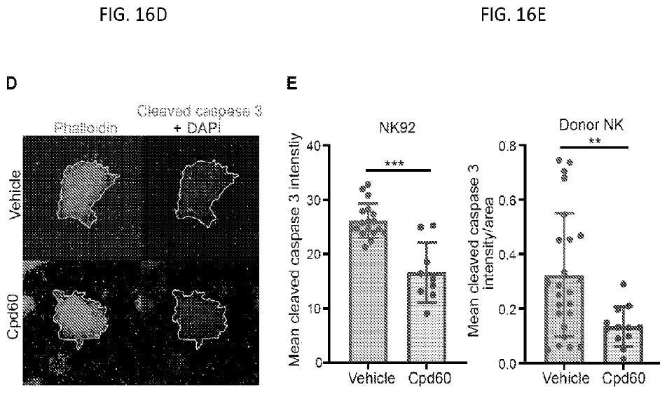

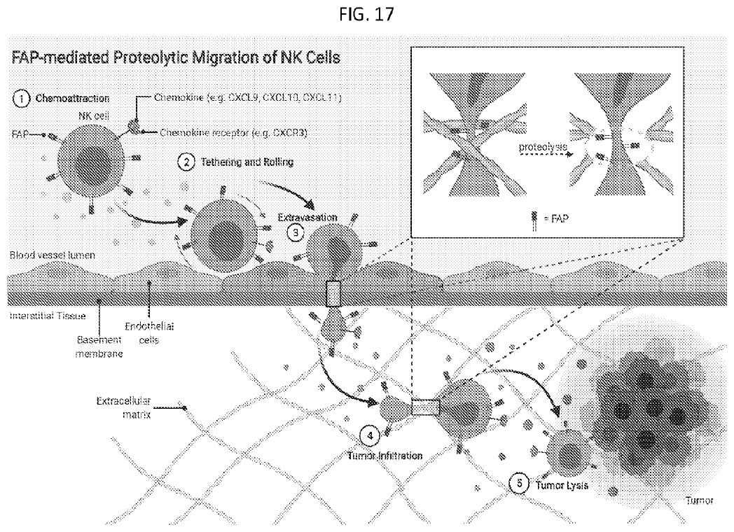

While the spheroid experiments suggest that FAP inhibition reduces NK cell migration through a tumor-associated extracellular matrix, interpretation of these results was constrained because the pancreatic stellate cells (PSCs) incorporated into the tumor spheroids to produce matrix also express FAP. Therefore, we could not exclude the possibility that FAP inhibition reduced NK cell infiltration into tumor spheroids by inhibiting the FAP activity of PSCs. To address this issue, we assessed the effect of FAP inhibition on NK cell infiltration into PANC-1 clusters embedded in matrix. These culture systems did not contain PSCs; the only FAP expressing cells present were the NK cells. We also investigated the effect of FAP inhibition on NK cell infiltration into PSC clusters embedded in matrix. To accomplish this, we plated 1,000 PANC-1 or PSCs in low-adhesion U-bottom plates and allowed them to form clusters for 24 hours. We then embedded the clusters in matrix that consisted of 80% collagen/20% Matrigel and NK92-GFP cells, and added either 10 uM Cpd60 or vehicle to the media. We live imaged the cocultures for 24 hours, capturing images every 30 minutes. Then we fixed the slides and stained for GFP by immunofluorescence to quantity the amount of NK cell infiltration into the clusters (FIG. 16A). FAP inhibition had no effect on cluster size. FAP inhibition significantly reduced NK92-GFP cell infiltration into PANC-1 and PSC clusters embedded in matrix (FIG. 16B and FIG. 16C). To determine if this reduced NK cell infiltration was accompanied by reduced tumor cell lysis we repeated the PANC-1 and NK92 coculture experiment and stained the cells for phalloidin and cleaved caspase 3 to identify apoptotic cells. Using the phalloidin stain we outlined the PANC-1 cell cluster, and then transposed the outline onto the cleaved caspase 3 images and quantified the intensity of cleaved caspase 3 within PANC-1 cell clusters (FIG. 16D). We found that FAP inhibition significantly reduced the amount PANC-1 cell apoptosis (FIG. 16E) in 3D cultures, despite having no effect on PANC-1 cell apoptosis in 2D cell cocultures. To determine if FAP inhibition also reduced donor NK cell migration and tumor lysis, we repeated these experiments with NK cells from two donors. Since the range of PANC-1 cluster areas in the donor NK cell experiment was much wider than the range in the NK92 experiment (10-208 versus 12-70) we normalized the intensities in the donor NK cell experiment to the area of the cluster. In agreement with the NK92 cell experiments, FAP inhibition reduced donor NK cell lysis of PANC-1 cells in 3D (FIG. 16E) but not 2D. This demonstrates that FAP inhibition does not alter target cell lysis through direct impacts on NK cell cytotoxicity but rather via modulation of NK cell migration through matrix. Therefore, quantitatively increasing FAP expression by NK cells may increase their capacity to invade through tumor matrix and promote the anti-tumor properties of human CAR-NK cells that target the well-characterized PDAC tumor-associated antigen, mesothelin.

Discussion

Here we show that FAP is expressed by human natural killer (NK) cells, and regulates NK cell migration, extravasation and tumor infiltration (FIG. 17). This observation adds to current understanding of NK cell migration and tissue infiltration, and describes a mechanism for NK cell extravasation from blood vessels. We additionally show that reduced tumor infiltration reduces tumor cell lysis, confirming the importance of FAP-based migratory mechanisms in the anti-cancer activity of NK cells. This work therefore reveals novel insights into FAP biology and NK cell biology and has important implications for emerging NK cell-focused therapeutic strategies.

For extravasation or tissue invasion, cells must penetrate the basement membrane and interstitial tissue, where they are confronted by 3D extracellular matrix (ECM) that provides a substrate for adhesion and traction, as well as biomechanical resistance. For cells to navigate through the ECM, which can offer narrow or non-existent pores for passage, leukocytes must adopt contracted shapes to traffic effectively. Excessive cellular deformation can result in nuclear rupture that causes genomic damage, long-term genomic alterations and limited cellular survival. To circumvent nuclear damage, some cells employ proteolytic digestion to widen pores in the ECM (Kameritsch & Renkawitz, 2020). Although proteolytic migration is considered less common in leukocytes versus other cell types, it has been documented. Zebrafish neutrophils and macrophages use proteolytic digestion for basement membrane transmigration (van den Berg et al., 2019). Human neutrophils secrete elastase, a serine protease, to facilitate their endothelial transmigration (Kurz et al., 2016).

In comparison to other immune cell types, there are few studies investigating the physical mechanisms driving NK cell migration. Decades-old research demonstrated that mouse and rat NK cell migration through Matrigel was dependent on matrix metalloproteinases (MMPs) (M. H. Kim et al., 2000; Kitson et al., 1998; Zeng et al., 1996). More recent studies have used more physiologic models. Putz et al. showed that heparinase regulated mouse NK cell infiltration into murine tumors (Putz et al., 2017). Prakash et al. showed that granzyme B released from murine cytotoxic lymphocytes, including NK cells, enhanced lymphocyte extravasation via ECM remodeling, although it did not affect interstitial migration. They confirmed that a granzyme B inhibitor reduced human donor T cell transmigration through a Matrigel coated semi-permeable membrane (i.e. Boyden chamber assay) (Prakash et al., 2014). Although these authors did not assess changes in human donor NK cell migration in response to a granzyme B inhibitor, it is reasonable to assume it would be similar to that of T cell migration since both cell types express and release granzyme B. However, our finding that FAP is expressed exclusively in human NK cells, and not in murine NK cells or other human immune cell types, suggests that some migratory mechanisms can be cell-type and species-specific. Unlike these previous studies that investigated either extravasation or tumor infiltration, we investigated both and found that NK cells use the same proteolytic migration strategy for basement membrane degradation/extravasation as well as tumor tissue infiltration. We further prove that defects in proteolytic migration directly impair the ability of NK cells to lysis malignant cells. FAP is a well-studied protein. Although once thought to be restricted to activated fibroblasts, FAP expression has been found in additional cell types such as epithelial tumors (Iwasa et al., 2003; Kelly et al., 1998; Mori et al., 2004), melanocytes (Monsky et al., 1994) and macrophages (Arnold et al., 2014; Tchou et al., 2013). In non-immune cells, FAP enhances cellular invasion (Ghersi et al., 2006; Kennedy et al., 2009; Monsky et al., 1994; Ruan et al., 2018; Wäster et al., 2011). The role of FAP in macrophages is less clear. Arnold et al. showed that in murine tumors there is a an FAP+ minor sub-population of immunosuppressive F4/80hi/CCR2+/CD206+M2 macrophages. While this study highlighted how FAP+ macrophages affect tumor growth, FAP's function in these macrophages was not described (Arnold et al., 2014). Tchou et al. identified FAP+CD45+ cells in human breast tumors by immunofluorescence. They then used flow cytometry to demonstrate that some of these FAP+CD45+ cells were CD11b+CD14+MHC-II+ tumor associated macrophages. Since the flow cytometry panel used to categorize these FAP+CD45+ cells consisted of only macrophage markers, those data do not exclude the possibility that some of the FAP+CD45+ tumor cells were NK cells. In contrast to that study, we did not identify FAP expression in human macrophages (CD14+ cells) (FIG. 2.6D). However, we examined circulating cells, as opposed to cells in the tumor microenvironment. Future studies are needed to further categorize FAP expression in tumor immune cell populations, presumably using multicolor immunofluorescent staining, since we were unable to detect FAP expression by NK cells using cell surface-based flow cytometry approaches. Additionally, more studies are needed to determine the function of FAP in these FAP+ tumor macrophages to determine if it enhances proteolytic migration similar to FAP's function in NK cells which we described here and as shown exemplarily in FIG. 17.

The findings that human NK cells express FAP has several clinical implications for FAP-targeted therapies. For example, an anti-FAP/IL-2 fusion protein is currently in clinical trials (NCT02627274). The proposed mechanism of action of this drug is that it targets IL-2 to FAP expressing tumor stroma, thereby limiting on-target, off-site toxicities associated with IL-2 cytokine therapy. Our findings that FAP is expressed on the NK cell surface suggests that and anti-FAP/IL-2 fusion protein may also target IL-2 directly to NK cells, enhancing NK cell activation and potentially tumor clearance. Since we were unable to detect FAP on the NK cell surface by flow cytometry, it is plausible that the anti-FAP construct in the anti-FAP-IL-2 fusion protein targets a similar epitope as the anti-FAP antibody we used, and therefore would not target IL-2 to NK cells. The inability to detect FAP on NK cells by flow may be a byproduct of masked epitope or altered FAP structure, which would render nearly all anti-FAP antibodies unusable, or due to an antibody-specific problem meaning other anti-FAP antibodies would bind FAP on NK cells. Future studies are needed to determine if the anti-FAP/IL-2 fusion protein currently in clinical trials can bind to FAP on the NK cell surface.

Anti-FAP CAR therapies are also in development to treat conditions such as cardiac fibrosis (Aghajanian et al., 2019), malignant pleural mesothelioma (Schuberth et al., 2013), lung adenocarcinoma (Kakarla et al., 2013) and other cancers (Santos et al., 2009). Our data suggest that anti-FAP CAR cells may also be useful in NK cell malignancies such as aggressive NK-cell leukemia if the anti-FAP portion was able to bind FAP on NK cells. There are potential caveats to the clinical use of anti-FAP CAR T cells. In one study, anti-FAP CAR T cells failed to regulate murine tumor growth and induced lethal bone toxicity and cachexia, potentially through the lysis of multipotent bone marrow stromal cells (Tran et al., 2013). It is plausible that an anti-FAP CAR T cell could induce NK cell lysis, resulting in NK cell leukopenia in humans only, therefore this toxicity would be missed in preclinical murine models. For cancer immunotherapy, an ideal anti-FAP CAR would be engineered to target FAP expression by fibroblasts and spare NK cells. Our findings that the anti-FAP antibody we used had variable binding to fibroblasts (i.e. PSCs) versus NK cells suggest this type of anti-FAP CAR engineering is feasible. It should be noted that Gulati et al. performed the first-in-human trial of an anti-FAP CAR T cell therapy, and demonstrated that a FAP CAR T cell therapy induced stable disease for 1 year in a patient with malignant pleural mesothelioma without any treatment-terminating toxicities (Schuberth et al., 2013). Our findings that FAP regulates NK cell tissue infiltration has clinical implications. FAP inhibitors, such as Cpd60, could reduce NK cell infiltration into tissues in diseases characterized by excessive NK cell content. These results also imply the potential value of engineering NK cells that overexpress FAP, enhancing NK cell tumor infiltration, which in turn would enhance tumor cell lysis.

Other strategies aimed at enhancing NK cell infiltration into tumors rely on manipulating chemokine/receptor pathways. One approach is to process NK cells in such a way that they have enhanced migratory phenotypes. For example, Wennerberg et al demonstrated that ex vivo expanded NK cells express higher levels of chemokine receptor CXCR3 than unexpanded NK cells. The expanded NK cells in turn had increased migration towards CXCL10 expressing melanomas (Wennerberg et al., 2014). They suggested that autologous NK cell therapy could be improved by expanding the NK cells prior to reinjection to enhance tumor homing. Another approach is to engineer NK cells to enhance their migration. Kremer et al engineered NK cells to overexpress CXCR2, a chemokine receptor. They showed that CXCR2 overexpressing NK cells had enhanced trafficking towards and lysis of renal cell carcinoma cells in vitro (Kremer et al., 2017). These findings suggest that strategies to enhance NK cell migration are both feasible and may have clinical applicability. However, chemokine pathway-altering strategies have built-in limitations. They require not only elevated expression of the chemokine receptor on NK cells, but also secretion and maintenance of chemoattractants by the tumor. Additionally, many chemoattractants recruit multiple immune cell types, including immunosuppressive cells. For example, CXCL10 is a chemoattractant for cytotoxic T lymphocytes and NK cells, but also for regulatory T cells (Lunardi et al., 2015). We postulate that the ideal migration-altering therapeutic approach would increase cytotoxic immune cell infiltration in tumor masses, without influencing or even reducing immunosuppressive immune cell content in the TME. Since inhibiting FAP reduces NK cell tumor infiltration and lysis, we therefore speculate that the inverse is true and that engineering NK cells to overexpress FAP, either in autologous NK cell or NK CAR-NK therapies, could increase NK cell tumor infiltration and lysis. This approach is independent of tumor-associated factors, such as chemoattractant secretion, and would not be expected to induce the infiltration or expansion of immunosuppressive cell populations into the tumor microenvironment. Since proteolytic migration is required for NK cell killing of malignant cells, the ability to alter protease expression or activity to enhance NK cell tumor infiltration represents a potentially promising approach to altering NK cell anti-tumor activity. Future studies are needed to explore the benefit of FAP-overexpressing NK cells in preclinical models and in clinical studies, and to determine what, if any, toxicities they induce.

This work also demonstrates the feasibility of studying human NK cell migration using physiologically relevant approaches in model systems. Human immune cell intravasation/extravasation studies often rely on artificial endothelial cell membranes. By using a zebrafish model, we were able to visualize and quantify human NK cell migration and extravasation in vivo. Van den Berg et al. demonstrated zebrafish models could be combined with advanced microscopy techniques, such as correlative light and electron microscopy, to visualize zebrafish immune cell extravasation (van den Berg et al., 2019). We have built upon this work and demonstrated that human NK cells can be inoculated into zebrafish embryos without apparent toxicities to the cells or the fish. We believe this model fills a current gap in available methodologies to investigate human immune cell migratory phenotypes in vivo. We also demonstrated that heterotypic spheroids, comprised of stromal producing cells and cancer cell lines, can be used to assess the impact of tumor matrix on immune cell migration complementing the less physiologic yet more controllable approach of embedding cells in 3D matrices.

The FAP functional studies described here would be improved with the application of FAP knock out NK cells. Because the generation of knock out NK cells is technically challenging, we believe the specific small molecule FAP inhibitor, Cpd60, represents a reasonable alternative to genetic knockout and also demonstrates that small molecules targeting FAP can alter NK cell function. In this study we have demonstrated that human NK cells express FAP and that human NK cells use FAP for migration, extravasation and tumor infiltration. These findings further the understanding of FAP biology and NK cell migration strategies. These results have meaningful implications for FAP-targeting therapies currently in development and represent novel mechanisms that can be exploited to alter NK cell biology for clinical benefit.

FAP Expression in NK Cells

Materials and Methods

Cell Pellets, Lines, and Cultures

Primary human PSCs (ScienCell, cat #3830) were maintained on plastic and passaged every 1-3 days in stellate cell medium (ScienCell, cat #5301). For all experiments, PSC passage 5-9 was used. All human NK cell lines (NK92, NKL, YT and KHYG-1) and murine NK cell lines (LNK) were kindly provided by Dr. Kerry S. Campbell (Fox Chase Cancer Center, Philadelphia, PA). The NK92-GFP expressed GFP due to nucleofection with pmaxGFP according to manufacturer's protocol (Lonza, cat #VVCA-1001). All NK cell lines were cultured as previously described (24), tested for mycoplasma every 3-6 months and fingerprinted annually. (NKL could not be fingerprinted because it has no published profile). PANC-1 cells were cultured in 10% FBS in DMEM. The cell pellets of cell lines tested for FAP expression by western blot (Jurkat, HuT 78, CCRF-CEM, Ramos, Namwala, IM-9, mono-mac 6, THP-1, U-937, Swiss3T3, RAW264.7, JAWSII, P815, BW5147.3, EL4 and A-20) were obtained from the Georgetown Lombardi Comprehensive Cancer Center Tissue Culture Shared Resource.

Healthy Donor Derived Cells

Fresh healthy donor NK cells were purchased from AllCells with either CD56 positive selection or CD56 negative selection (Allcells, cat #PB012-P or PB012-N). For 2D migration experiments, NK cells were enriched from peripheral blood using RosetteSep (StemCell Technologies) from healthy adult donors. T cells, B cells and monocytes were isolated from PBMCs (Allcells) using Mojosort magnetic cell separation system from Biolegend via CD3 positivity (Biolegend, cat #480133), CD19 positivity (Biolegend, cat #480105), CD14 positivity (Biolegend, cat #480093). PBMC purity was assessed using flow cytometry: CD3-APC (Biolegend, cat #300411), CD14-BV421 (Biolegend, cat #325627), CD45-FITC (BD Bioscience cat #347463), CD56-PE (BD Bioscience, cat #555516), CD20-PE (BD Bioscience, cat #555623). For donor NK cell lysis of PANC-1 clusters, primary donor NK cells were purchased from Allcells then expanded using irradiated K562-4-1BBL-mbIL-21 (names “CSTX002”) cells kindly provided by Dr. Dean Lee according to his protocol (25).

FAP Activity Assay

One day prior to assay, 5,000 PSCs/well were added to 96 well flat clear bottom white polystyrene TC-treated microplates (Corning, cat #3610). The following day, PSC media was aspirated off and 50 uL of NK92 cells (lacking GFP) were added to each well containing PSCs at a 4:1 E:T ratio and incubated overnight at 37° C. 100 mM stock of dipeptidylpeptidase substrate (Acetyl-Aka-Gly-Pro-AFC) (Anaspec, CatAS-24126) was made by resuspending lyophilized substrate in DMSO. On the day of the assay, DMSO stock was then diluted 1:1000 in FAP activity assay buffer (50 mM Tris-BCl, 1 M NaCl, 1 mg/mL BSA, pH 7.5). A standard curve was generated using rFAP (R&D systems, 3715-SE-010). 50 uL of rFAP standard was added to wells in triplicate. 50 uL of substrate was added to each well and the plate was incubated for 5 minutes at 37° C. The plate was read on a PerkinElmer EnVision Multimode Plate Reader with 390-400 nm excitation and 580-510 nm emission wavelengths. The final concentration of FAP per well was calculated using the standard curve. Data were compiled and assessed for statistical significance using GraphPad Prism 9.

PSC-NK92 Coculture Assay

PSCs were plated one day prior to assay at 100,000 cells/well in a 6 well collagen coated plate. NK92 cells were added at 1:1 or 4:1 effector to target (E:T) ratios and cocultured for 3-4 hours. Each well contained 50% v/v NK and PSC media and 1% v/v IL-2. Following incubation, nonadherent cells were collected. Adherent cells were washed 2× with PBS and then trypsinized with 0.05% trypsin. After detachment trypsin was quenched with equal volume PSC media and cells were collected, pelleted and washed 2× with PBS then resuspended in 600 uL of 1% BSA. Cells were immediately sent for nonsterile flow sorting of GFP+ from GFP− using the BDFACS Aria Ilu cell sorter in the Georgetown Lombardi Comprehensive Cancer Center Flow Cytometry and Cell Sorting Shared Resource (FCSR).

RNA Isolation and rt-qPCR

RNA was isolated using the PureLink RNA Mini Kit (Ambion, cat #12183020). The RNA concentration was measured using NanoDrop 8000 (Thermo Fisher Scientific). cDNA was generated from 20-100 ng of RNA using the GoTaq 2-step RT-qPCR System (Promega, cat #A6110). qPCR was performed with SYBR Green on a StepOnePlus real-time PCR system (Applied Biosystems). Gene expression was normalized to HPRT and analyzed using 1/ΔCt method.

### Primers Sequences:

FAP
(F: ATGAGCTTCCTCGTCCAATTCA;
R: AGACCACCAGAGAGCATATTTTG)
HPRT:
(F: GATTAGCGATGATGAACCAGGTT;
R: CCTCCCATCTCCTTCATGACA)

Western Blot

Western blots were performed as previously described (24). Western blots were conducted using anti-FAP (ab207178, abcam) at concentrations of 1:1000 diluted in 5% milk in PBST. Secondary antibody was anti-rabbit IgG, HRP linked (Cell Signaling, cat #7074S) at 1:1000. Antibody was validated with additional anti-FAP antibodies (MyBiosource, cat #MBS303414 and abcam, car #ab53066). GAPDH antibody (Cell Signaling, cat #5174S) was used at 1:10,000. The secondary antibody was anti-rabbit IgG, HRP linked (Cell Signaling) used at 1:5000. Chemiluminescent substrate (Pierce, cat #32109 or cat #34094) was used for visualization.

FAP Flow Cytometry

Cell pellets were collected via centrifugation at 1000 rpm for 5 mins. Cells were washed 2× with 1×PBS and resuspended in 100 uL staining buffer (1% BSA in PBS). To test western blot anti-FAP antibodies, first 5 uL of anti-FAP antibody was used (either ab207178 or ab53066) incubated for 30 min at 4 degrees in the dark then washed twice with staining buffer. Then 2 uL of secondary antibody (Goat F(ab′)2 Anti-Rabbit IgG(H+L) Alexa Fluor 647 (southern biotech, cat #4052-31)) was added, incubated for 30 min at 4 degrees in the dark then washed twice with staining buffer. After this failed to work we removed the need for secondary antibody by conjugating ab207178 to APC using a lightning link conjugation kit (abcam, cat #ab2018071). Various concentrations of ab207178-APC antibody were tested. For successful flow, 1 uL of human Fc block (BD Pharmingen, cat #564219) was added and incubated at 4° C. for 45 minutes. 4 uL of 0.25 mg/mL sheep anti-human FAP antibody (R&D systems, cat #AF3715) or 0.5 uL of 2 mg/mL sheep IgG control (R&D systems, cat #5-001-A) was added and cells incubated at 4° C. for 30 minutes, vortexing half way through. Cells were washed 2× with staining buffer then resuspended in 100 uL staining buffer. 2 uL of PE-conjugated donkey anti-sheep secondary was added (R&D systems, cat #F0126) and incubated at 4° C. for 30 minutes in the dark, vortexing half way through. Cells were washed 2× with staining buffer then resuspended in 600 uL staining buffer. Samples were run in the Georgetown Lombardi Comprehensive Cancer Center Flow Cytometry Cell Sorting Shared Resource using BD LSRFortessa. Analyses were performed using FlowJo (v10.4.1).

Cell Surface Biotinylation

Cell surface biotinylation of NK92, NKL, YT and KHYG-1 cells was performed with the Pierce Cell Surface Protein Isolation kit (Thermo Scientific, cat #89881) according to the manufacturer's protocol. In brief, 4×10S cells were pelleted and washed with cold PBS then incubated with EZ-LINK Sulfo-NHS-SS-biotin for 30 min at 4° C. followed by the addition of a quenching solution. Another 1×10⁶ cells were collected and saved for total cell western blotting. Cells were lysed with lysis buffer (500 μL) containing the cOmplete protease inhibitor cocktail (Roche, cat #11697498001). The biotinylated surface proteins were excluded with NeutrAvidin agarose gel (Pierce, 39001). Samples were diluted 50 ug in ultrapure water supplemented with 50 mM DTT. Lysates were subjected to Western blotting with the anti-FAP antibody described above.

Computational Analysis

NK lymphoma and cell line gene expression was downloaded from GEO (GEO accession GSE19067) (26) using R version 3.6.2 and read using affy in Bioconductor (27). Non-NK cell samples were excluded from analysis. Heatmap was created using ComplexHeatMap version 2.1.1 (28). Correlation analysis was performed using limma in Bioconductor (29). Gene set enrichment analysis was performed using GO enrichment (30).

2D NK Migration Studies

2D migration studies were done as previously reported (31, 32). In brief, EL08.1D2 stromal cells were grown to a confluent monolayer on flat-bottomed 96 well ImageLock plates (Essen Bioscience) pre-coated with 0.1% gelatin (Stemcell Technologies). 10 uM of Cpd60 in RPMI media was added to the chamber 15 min before imaging. Freshly isolated human NK cells were imaged in 96-well on the IncuCyte ZOOM Live-Cell Analysis System (Essen Bioscience) at 37° C. every 2 min in the phase-contrast mode (10× objective). Tracking of live cells was done using the manual tracking feature in Fiji (33). Tracks were plotted using the Chemotaxis plugin of FIJI. Cells that were in the field of imaging for fewer than two frames were discarded, as were cells which were non-adherent or floating. EL08.1D2 cells were used as de facto fiducial markers to ensure that neither they or the microscope stage was drifting and causing apparent NK cell movement. Length and displacement measurements were derived directly from tracked cells and graphed using GraphPad software. Velocity data was obtained by dividing the total track length by the time of imaging.

Zebrafish Studies

Zebrafish studies were conducted in accordance with NIH guidelines for the care and use of laboratory animals and were approved by the Georgetown University Institutional Animal Care and Use Committee. Zebrafish husbandry, injections, and mounting was performed by the Georgetown-Lombardi Animal Shared Resource. Two day post fertilization stage Tg(kdrl:mCherry-CAAX) embryos were anesthetized with 0.016% tricaine (Sigma-Aldrich, St. Louis, MO, USA) in fish water (0.3 g/L Sea Salt, Instant Ocean, Blacksburg, VA) and were injected with 100-200 NK92-GFP cells into the precardiac sinus using an air driven Picospritzer II microinjector (General Valve/Parker Hannifin) under a stereoscope. Following injection, embryos with cells in the caudal hematopoietic tissue were selected for analysis and mounted in 1.5% agarose plus 0.011% tricaine in fish water. Fish were maintained at 33° C. until imaging. Confocal imaging was performed on a Leica SP8 AOBS microscope in the Georgetown-Lombardi Microscopy Shared Resource. Widefield fluorescent imaging was performed on a Keyence BZ-X inverted microscope. Images were taken at 10× across multiple z-stacks. Z-stack images were compressed using full focus and haze reduction in Keyence BZ-X software. NK extravasation quantification was performed by counting the number of GFP cells outside red vasculature. NK extravasation quantification was performed blinded to the treatment conditions. Graphs of resulting data and statistical analysis was generated using Graphpad Prism 9.

Spheroid Studies

PSC or PANC-1 spheroids were generated by plating 10,000 cells in a 0.1% agarose coated U-bottomed 96-well plate. PSC+PANC-1 spheroids were generated by plating 5,000 cells of each cell type. Aggregation was promoted by centrifuging the cells at 1000 rpm for 5 minutes. Cells incubated overnight at 37° C. The next day, 1,000 NK92-GFP cells were added per well and incubated for 4 hours at 37° C. Spheroids were imaged using the Olympus IX-71 Inverted Epifluorescent Microscope at 5×. Images were analyzed in FIJI. All images underwent identical contrast enhancement and background reduction. Then a line was drawn from spheroid edge to spheroid center and GFP intensity along that line was measured. Graphs of resulting data and statistical analysis were generated in Graphpad Prism 9.

3D Cluster Studies

3D clusters were generated, embedded and stained as previously described (34, 35). In brief, clusters were generated by plating 1,000 cells per well into 96-well Nunclon Sphera low adhesion plates (Thermo Scientific, cat #174925) and incubated overnight at 37° C. The following day, 6 clusters were embedded into an ECM containing 2,000 NK cells were plated into one well of a Nunc Lab-Tek II 8-well chamber slide (ThermoScientific, cat #154534PK). To ensure equal distribution of NK cells in Matrigel, the NK cells were first suspended in the Matrigel stock, which was then aliquoted for individual cluster embedding. The ECM mixture consisted of 20% growth factor reduced Matrigel (Corning, 10-12 mg/ml stock concentration, #354230) and 80% rat tail collagen type I at 3 mg/mL (Gibco, A1048301). Cells were either imaged for the following 24 hours every 30 minutes using a Zeiss LSM800 scanning confocal microscope enclosed in a heated chamber supplemented with CO₂ or allowed to incubate overnight at 37° C. After 24 hours, cells in matrix were fixed with 5.4% formalin for 1 hour, permeabilized with 0.5% Triton-X and blocked using goat serum. For invasion assays, NK-92-GFP cells were stained with anti-GFP (ThermoFisher, cat #A-11122). For the cell lysis assays, clusters were stained using anti-cleaved caspase 3 (Cell Signaling, cat #9661). Hoechst 33342, phalloidin, and secondary antibodies labeled with Alexa Fluor 488 nm, 546 nm, 647 nm, or 680 nm (Invitrogen) were used.

Annexin V NK Cell Lysis Study

One day prior to assay, PSCs were stained with DiI. If donor NK cells were used, they were stained with DiO prior to the experiment. Cells were then plated as described for the PSC-NK92 coculture assay. Following incubation period of 4 hours, all cells from a single well were collected and washed 2× with PBS. Samples were then processed by the FCSR using the Alexa Fluor 647 Annexin V and Sytox Blue staining (Biolegend). Flow data were analyzed using FloJo (v10.4.1) and statistics was performed using GraphPad Prism 9.

Results

Human Natural Killer Cells Express Catalytically Active Fibroblast Activation Protein (FAP)

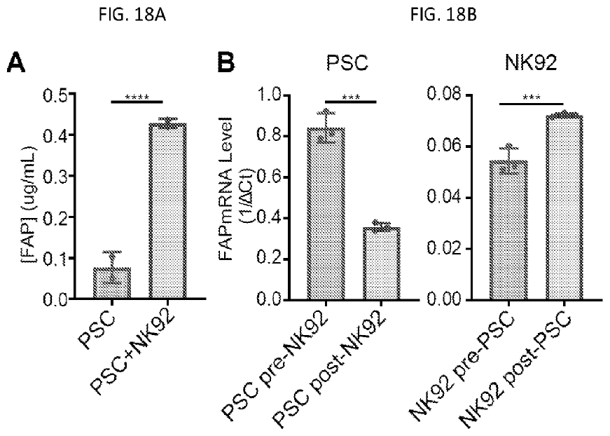

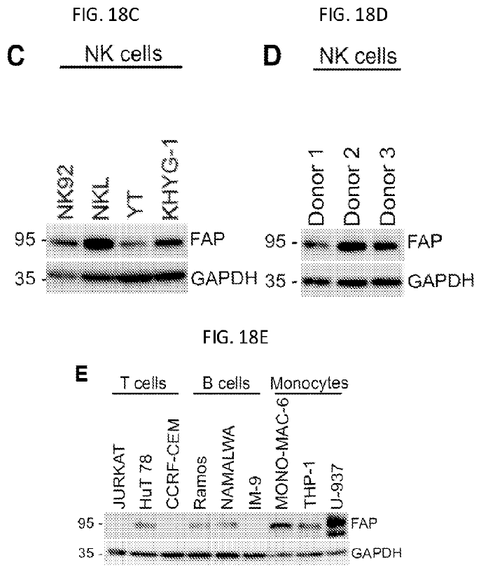

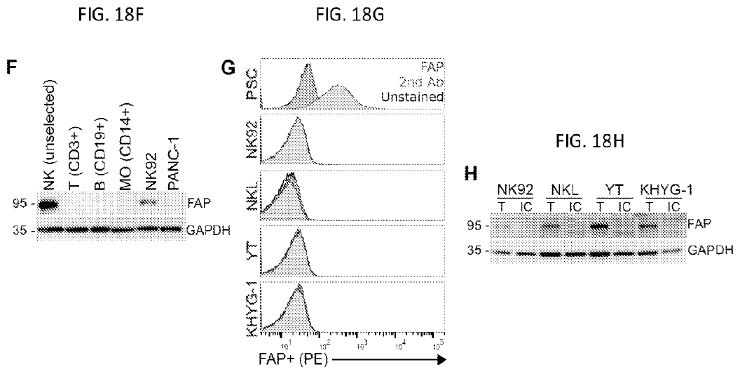

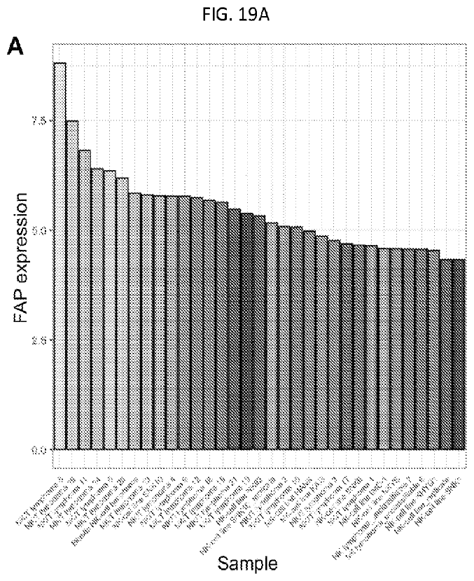

We used pancreatic ductal adenocarcinoma (PDAC) as a model since it is characterized by extensive stroma that physically excludes immune cells (36). In PDAC, activated pancreatic stellate cells (PSCs) produce fibroblast activation protein (FAP) (37). We began by exploring the impact of NK cells on FAP expression by PSCs and cocultured primary PSCs with the human NK cell line NK92. Coculture of PSC with NK92 cells led to a four-fold increase in FAP activity compared to PSCs cultured alone as determined by a fluorescent peptide substrate FAP activity assay (FIG. 18A). However, this coculture experiment did not distinguish which cells produced the FAP (i.e. NK cells or PSCs). To address this, we cocultured PSCs with GFP expressing NK92 cells, FACS separated the two cell types and performed rt-qPCR for FAP expression in each cell population. Surprisingly, after 4 hours of coculture the PSCs possessed significantly reduced FAP expression, while the NK92 cells not only expressed FAP, but showed significantly increased FAP expression after coculture with PSCs (FIG. 18B).

Since NK cells are not known to produce FAP, we confirmed FAP expression at the protein level in NK92 cells and three additional human NK cell lines: NKL, YT and KHYG-1 (FIG. 18C). To exclude the possibility that FAP expression was specific to NK cell malignancies, we assessed FAP expression in NK cells isolated from PBMCs of five different healthy human donors and found robust FAP expression in all donor NK cells (FIG. 18D). To determine if additional human immune cell types express FAP, we assessed multiple different human B, T and monocyte cell lines for FAP expression by western blot and found heterogeneous protein expression (FIG. 18E). This cell-line specific FAP protein expression was consistent with FAP mRNA expression as determined by analysis of RNAseq data derived from the cancer cell line encyclopedia (38). While we saw heterogeneous expression of FAP in B, T and monocyte cell lines, we did not detect FAP expression in healthy donor PBMC-derived B cells (CD19+), T cells (CD3+), and macrophages (CD14+) (FIG. 18F). Thus, FAP expression in non-NK cell lines is likely driven by their malignant biology, since FAP can be upregulated during the process of malignant transformation (21).

Canonically, FAP is surface-expressed, so we attempted to detect FAP by flow cytometry. Anti-FAP antibodies used for western blot failed to detect FAP on the positive control cell line (PSCs). A polyclonal sheep anti-FAP antibody detected FAP on PSCs, but was unable to detect FAP on NK cells (FIG. 18G). This does not preclude the possibility that FAP is surface expressed on NK cells, but shows currently available anti-FAP antibodies are unable to detect FAP on the NK cell surface. To circumvent this, we turned to an antibody-independent means of detecting surface expression-surface protein biotinylation. We biotinylated cell surface proteins, and then excluded them from the cell lysate via magnetic separation. We then determined that FAP is present in total cell lysate but absent from the intracellular protein lysate (FIG. 18H), demonstrating that FAP is expressed on the NK cell surface. Due to the volume required to unbind biotinylated surface proteins from the magnetic beads, the surface protein lysate was too dilute to perform adequate western blot analysis. Faint bands of the appropriate size were observed when using the maximum volume (40 uL) the gel allowed. On NK cells, FAP may be structurally different or exist within a protein complex, rendering flow-based antibodies ineffective.

In NK Cells, FAP Gene Expression Correlates with Extracellular Matrix and Migration Regulating Genes

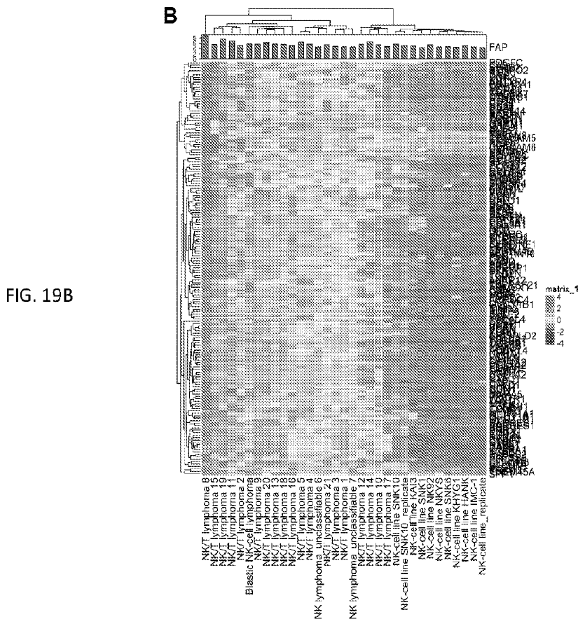

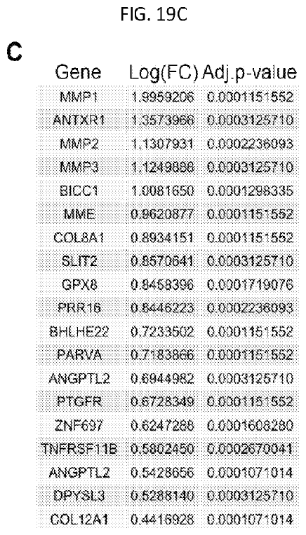

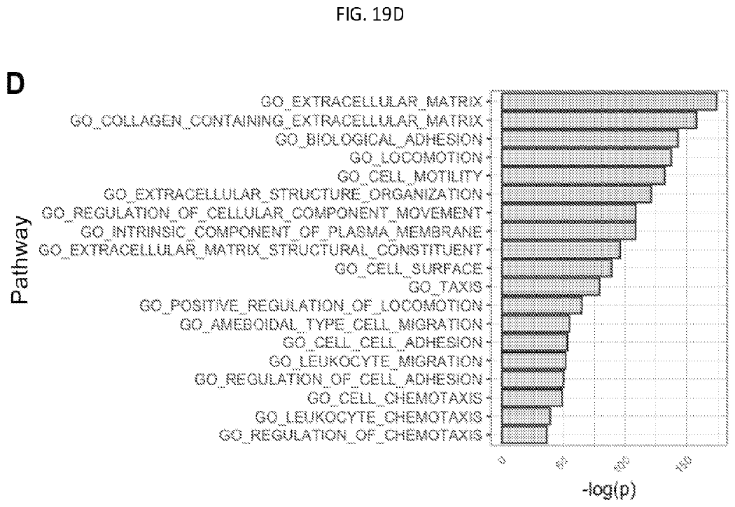

To determine FAP's function in human natural killer cells we employed computational approaches. In 2011, Iqbal et al. performed a gene expression array on multiple NK cell lymphoma samples and NK cell lines (26). Using these data, we assessed FAP expression in 22 NK cell lymphomas and 11 NK cell lines (FIG. 19A) and performed a correlation analysis to assess the genes that were most positively and negatively correlated with FAP expression (FIG. 19B). The top 19 genes that were most positively correlated with FAP expression are shown in FIG. 19C. We then performed GO enrichment analysis of these genes and determined that the pathways most positively correlated with FAP expression were related to extracellular matrix remodeling and cellular migration (FIG. 19D). This is consistent with the current understanding of FAP function, which is to cleave extracellular matrix components such as collagen and enhance cellular migration/invasion (21). It is also interesting that matrix metalloproteases (MMPs) were among the top 19 genes positively correlated with FAP expression. MMPs regulate rat, mouse and human NK cell migration into collagen or Matrigel in vitro (39-41). These data suggest that FAP may also regulate NK cell migration.

FAP Inhibition Reduces Primary Human NK Cell Migration

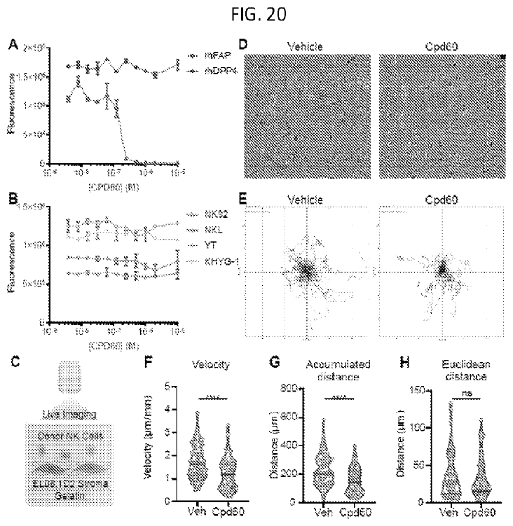

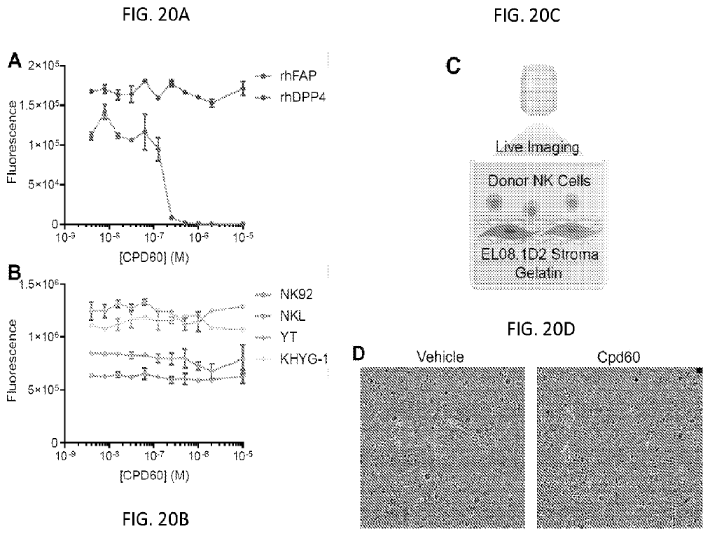

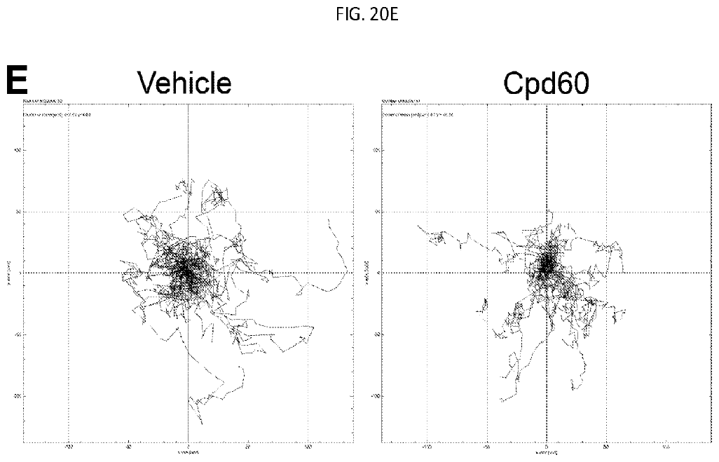

Based on the computational analysis, we hypothesized that FAP was expressed by human NK cells to enhance their migration. To test this hypothesis, we compared primary NK cell migration ex vivo in the presence and absence of a highly selective FAP-specific inhibitor-Cpd60. Cpd60 was designed to selectively inhibit FAP over other members of the prolyl oligopeptidase family S9. Cpd60's IC₅₀ for FAP is 0.0032 uM versus >100 uM for DPP4, >12.5 uM for DPP9, >100 uM for DPP2 and >1.8 for PREP (prolyl oligopeptidase) (42). We confirmed Cpd60 inhibited FAP but not FAP's most closely related protein, DPPIV (FIG. 20A). Cpd60 had no effect on NK cell viability (FIG. 20B). To monitor NK cell migration we cocultured primary NK cells with EL08.1D2 cells, which have previously been shown to support spontaneous NK cell migration (31, 43) and produce extracellular matrix (32), and live imaged them for 24 h capturing photos every 2 minutes (FIG. 20C). From these time-lapse videos we manually tracked NK cell migratory paths (FIGS. 20D and 20E). These experiments were repeated with NK cells from three different donors, with similar results. We found that FAP inhibition with Cpd60 significantly reduced NK cell velocity (FIG. 20F) and the accumulated distance traveled by NK cells (FIG. 20G) but had no significant impact on the Euclidian distance—the straight-line distance between the starting point and end point—traveled by NK cells (FIG. 20H).

FAP Inhibition Reduces NK Cell Extravasation In Vivo

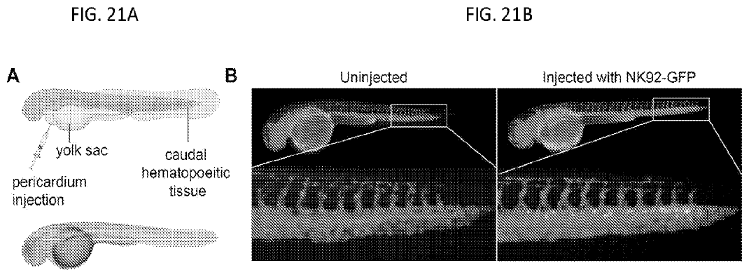

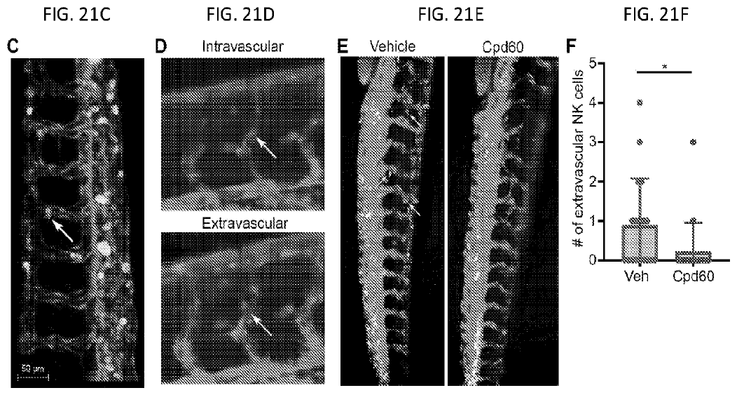

We next set out to determine if FAP altered NK cell migratory behaviors in vivo. Since we could not detect FAP expression in murine NK cells we opted to use zebrafish—a novel in vivo model that allows us to monitor human NK cell migratory behaviors in real-time. We injected NK92-GFP cells into the pericardium of Tg(kdrl:mCherry-CAAX)y171 zebrafish embryos that express endothelial membrane targeted mCherry (FIG. 21A). Immediately after injection, NK cells migrated via the circulation to the caudal hematopoietic tissue (FIG. 21B) hen gradually disseminating throughout the rest of the zebrafish vasculature. Using confocal live-imaging, which captured images approximately every 3 minutes, we captured an NK cell crawling along the inside of the blood vessel, searching for an appropriately sized pore just prior to extravasation (FIG. 21C). After confirming that human NK cells could migrate throughout and extravasate from zebrafish vasculature, we tested the effects of FAP inhibition on NK cell extravasation. Since fluorescent microscopy is amenable to imaging multiple fish simultaneously, we used fluorescent microscopy to quantify the effects of the FAP inhibitor Cpd60 on NK cell extravasation. We confirmed that the fluorescent microscope was capable of detecting NK cell extravasation (FIG. 21D), and then imaged the fish injected with NK92-GFP cells, half of the fish were bathed in 10 uM of Cpd60, and half of the fish were bathed in vehicle. Fish were imaged at 10× to visualize the entire fish. We found that FAP inhibition significantly reduced NK cell extravasation from the blood vessels (FIGS. 21E and 21F).

FAP Inhibition Reduces NK Cell Infiltration into Matrix Containing PDAC Tumor Spheroids

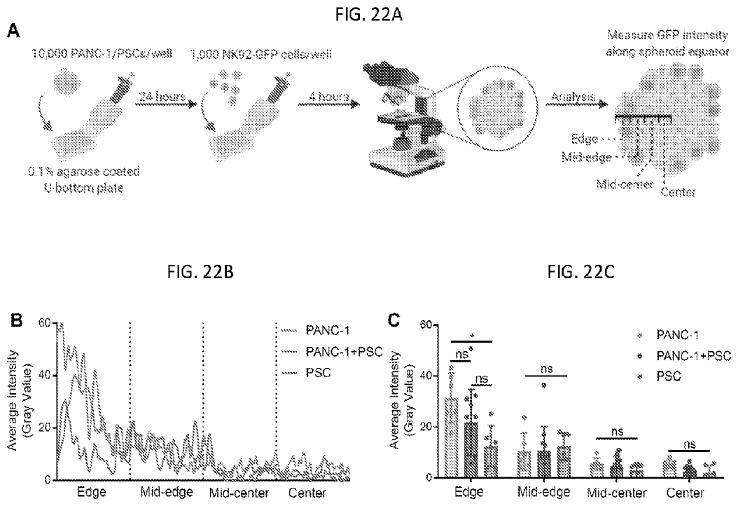

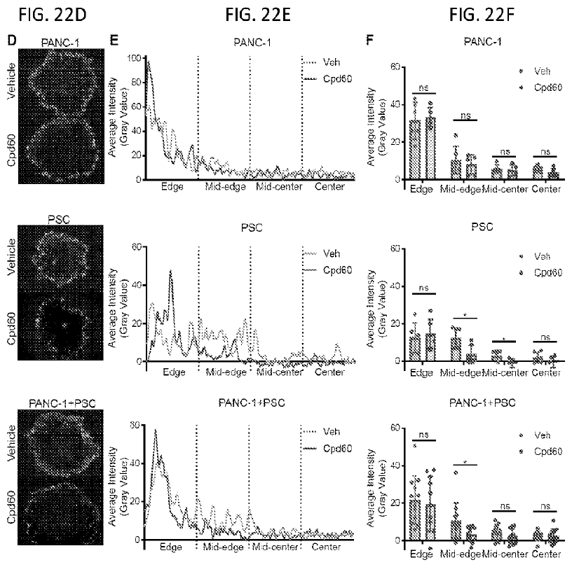

NK cells regulate tumor growth and viability, yet the mechanisms NK cells employ to migrate through dense tumor-related extracellular matrix is unknown. To determine if FAP activity affects NK cell infiltration into tumors we used tumor spheroid models of PDAC generated from the PDAC cell line PANC-1 and primary pancreatic stellate cells (PSCs, PSCs comprise the majority of cancer-associated-fibroblasts in PDAC tumors). Homogeneous PANC-1 tumor spheroids have minimal extracellular matrix but PSC and PSC+PANC-1 heterogeneous tumor spheroids contain rich stroma that contains extracellular components such as collagen and fibronectin (44, 45). We generated homogenous PANC-1 or PSC spheroid and heterogeneous PANC-1+PSC spheroids by plating 10,000 cells in a 0.1% agarose coated U-bottom plate and allowed the spheroids to form over 24 hours. After 24 hours, 1,000 NK92-GFP cells were added to the spheroids and 4 hours later the spheroid-NK cell cocultures were imaged using fluorescent microscopy. To assess the extent of NK cell infiltration into tumor spheroids, we measured GFP intensity along an equatorial line drawn from the surface of the spheroid in FIJI. The line drawn for each spheroid was the same length. We then divided this line into quarters, which we termed “edge”, “mid-edge”, “mid-center” and “center”, then averaged the intensity along those quarters (FIG. 22A).

As expected, NK cells infiltrated into PANC-1 spheroids more readily than they infiltrated PSC spheroids (FIGS. 22B and 22C). This is likely because PANC-1 spheroids do not contain extracellular matrix like PSC spheroids; PANC-1 spheroids thus lack the physical matrix barrier that impedes NK cell infiltration. We next assessed the impact of FAP inhibition on NK cell infiltration into homogenous PANC-1 spheroids, homogenous PSC spheroids, and heterogeneous PANC-1+PSC spheroids (FIGS. 22D, 22E and 22F). We found that 10 uM Cpd60 significantly reduced NK cell content in the mid-edge and mid-center regions of the PSC spheroids, and significantly reduced NK cell content in the mid-edge region of the PSC+PANC-1 spheroids, yet had no effect on NK cell content in any region of the PANC-1 spheroids. These results suggest that FAP regulates NK cell migration through tumors, but only in the presence of extracellular matrix.

FAP Inhibition Reduces NK Cell Infiltration into and Lysis of PANC-1 Cell Clusters Embedded in Matrix

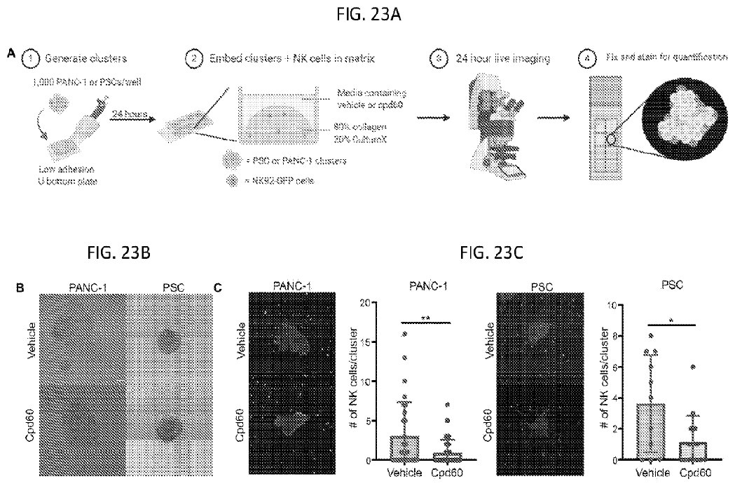

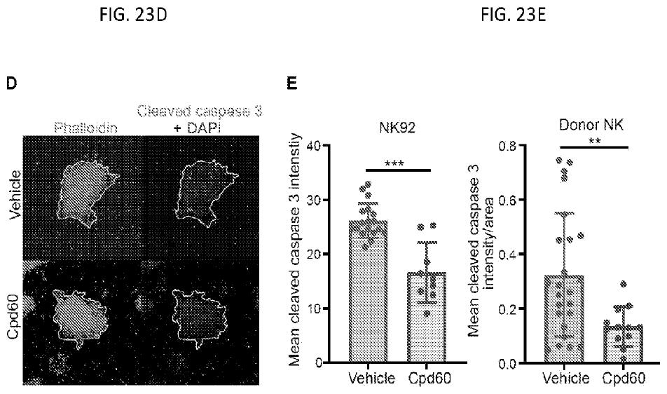

While the spheroid experiments shown in FIG. 5 suggest that FAP inhibition reduces NK cell migration through a tumor-associated extracellular matrix, interpretation of these results was constrained because the pancreatic stellate cells (PSCs) incorporated into the tumor spheroids to produce matrix also express FAP. Therefore, we could not exclude the possibility that FAP inhibition reduced NK cell infiltration into tumor spheroids by inhibiting the FAP activity of PSCs. To address this issue, we assessed the effect of FAP inhibition on NK cell infiltration into PANC-1 clusters embedded in matrix. These culture systems did not contain PSCs; the only FAP expressing cells present were the NK cells. We also investigated the effect of FAP inhibition on NK cell infiltration into PSC clusters embedded in matrix. To accomplish this, we plated 1,000 PANC-1 or PSCs in low-adhesion U-bottom plates and allowed them to form clusters for 24 hours. We then embedded the clusters in matrix that consisted of 80% collagen/20% Matrigel and NK92-GFP cells, and added either 10 uM Cpd60 or vehicle to the media. We live imaged the cocultures for 24 hours, capturing images every 30 minutes. Then we fixed the slides and stained for GFP by immunofluorescence to quantity the amount of NK cell infiltration into the clusters (FIG. 23A). FAP inhibition had no effect on cluster size. FAP inhibition significantly reduced NK92-GFP cell infiltration into PANC-1 and PSC clusters embedded in matrix (FIGS. 23B and 23C). To determine if this reduced NK cell infiltration was accompanied by reduced tumor cell lysis we repeated the PANC-1 and NK92 coculture experiment and stained the cells for actin using phalloidin and cleaved caspase 3 to identify apoptotic cells. Using the phalloidin stain we outlined the PANC-1 cell cluster, and then transposed the outline onto the cleaved caspase 3 images and quantified the intensity of cleaved caspase 3 within PANC-1 cell clusters (FIG. 23D). We found that FAP inhibition significantly reduced the amount PANC-1 cell apoptosis (FIG. 23E) in 3D cultures, despite having no effect on PANC-1 cell apoptosis in 2D cell cocultures. To determine if FAP inhibition also reduced donor NK cell migration and tumor lysis, we repeated these experiments with NK cells from two donors. Since the range of PANC-1 cluster areas in the donor NK cell experiment was much wider than the range in the NK92 experiment (10-208 versus 12-70) we normalized the intensities in the donor NK cell experiment to the area of the cluster. In agreement with the NK92 cell experiments, FAP inhibition reduced donor NK cell lysis of PANC-1 cells in 3D (FIG. 23E) but not 2D. This demonstrates that FAP inhibition does not alter target cell lysis through direct impacts on NK cell cytotoxicity but rather via modulation of NK cell migration through matrix. Therefore, quantitatively increasing FAP expression by NK cells may increase their capacity to invade through tumor matrix and promote the anti-tumor properties of human CAR-NK cells that target the well-characterized PDAC tumor-associated antigen, mesothelin.

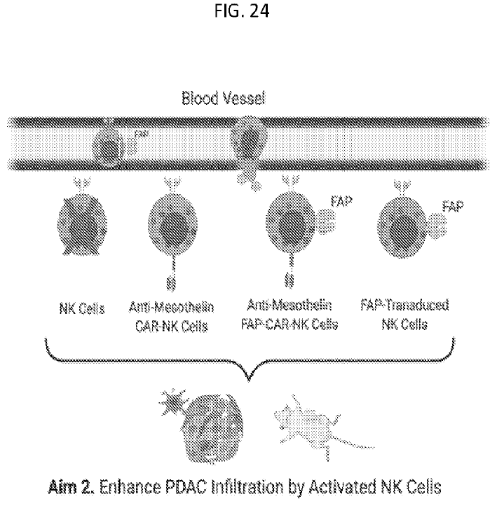

FIG. 24 shows a diagram of various NK cell types where increasing FAP expression can be used to enhance pancreatic ductal adenocarcinomas (PDAC) infiltration by activated NK cells.

Discussion

Here we show human natural killer (NK) cells express FAP, which regulates NK cell migration, extravasation and tumor infiltration. This observation adds to current understanding of NK cell migration and tissue infiltration, and describes a mechanism for NK cell extravasation from blood vessels. We additionally show that reduced tumor infiltration reduces tumor cell lysis, confirming the importance of FAP-based migratory mechanisms for the anti-cancer activity of NK cells. Therefore, this work reveals novel insights into FAP biology and NK cell biology and has important implications for emerging NK cell-focused therapeutic strategies.

For extravasation or tissue invasion, cells must penetrate the basement membrane and interstitial tissue. During this process they are confronted by 3D extracellular matrix (ECM) that provides a substrate for adhesion and traction, as well as biomechanical resistance. In order for cells to traffic effectively through the ECM, which can offer narrow or non-existent pores for passage, leukocytes must adopt contracted shapes. Excessive cellular deformation can result in nuclear rupture that causes genomic damage, long-term genomic alterations and limited cellular survival. To circumvent nuclear damage, some cells employ proteolytic digestion to widen pores in the ECM (20). Although proteolytic migration is considered less common in leukocytes versus other cell types, it has been documented. Zebrafish neutrophils and macrophages use proteolytic digestion for basement membrane transmigration (46). Human neutrophils secrete elastase, a serine protease, to facilitate their endothelial transmigration (47).

In comparison to other immune cell types, there are few studies investigating the physical mechanisms driving NK cell migration. Decades-old research demonstrated that mouse and rat NK cell migration through Matrigel was dependent on matrix metalloproteinases (MMPs)(40, 48, 49). More recent studies have used more physiologic models. Putz et al. showed that heparinase regulated mouse NK cell infiltration into murine tumors (50). Prakash et al. showed that granzyme B released from murine cytotoxic lymphocytes, including NK cells, enhanced lymphocyte extravasation via ECM remodeling, although it did not affect interstitial migration. They confirmed that a granzyme B inhibitor reduced human donor T cell transmigration through a Matrigel coated semi-permeable membrane (i.e. Boyden chamber assay) (51). Although these authors did not assess changes in human donor NK cell migration in response to a granzyme B inhibitor, it is reasonable to assume it would be similar to that of T cell migration since both cell types express and release granzyme B. However, our finding that FAP is expressed exclusively in human NK cells, and not in murine NK cells or other human immune cell types (FIG. 1), suggests that some migratory mechanisms can be cell-type and species-specific. Unlike these previous studies that investigated either extravasation or tumor infiltration, we investigated both and found that NK cells use the same proteolytic migration strategy for basement membrane degradation/extravasation as well as tumor tissue infiltration. We further prove that defects in proteolytic migration directly impair the ability of NK cells to lyse malignant cells.

FAP is a well-studied protein. Although once thought to be restricted to activated fibroblasts, FAP expression has been found in additional cell types such as epithelial tumors (52-54), melanocytes (55) and macrophages (56, 57). In non-immune cells, FAP enhances cellular invasion (55, 58-61). The role of FAP in macrophages is less clear. Arnold et al. showed that in murine tumors there is a FAP+ minor sub-population of immunosuppressive F4/80^{hi}/CCR2⁺/CD206⁺M2 macrophages. While this study highlighted how FAP+ macrophages affect tumor growth, FAP's function in these macrophages was not described (56). Tchou et al. identified FAP+CD45+ cells in human breast tumors by immunofluorescence. They then used flow cytometry to demonstrate that some of these FAP+CD45+ cells were CD11b+CD14+MHC-II+ tumor associated macrophages. Since the flow cytometry panel used to categorize these FAP+CD45+ cells consisted of only macrophage markers, those data do not exclude the possibility that some of the FAP+CD45+ tumor cells were NK cells. In contrast to that study, we did not identify FAP expression in human macrophages (CD14+ cells) (FIG. 1F). However, we examined circulating cells, as opposed to cells in the tumor microenvironment. Future studies are needed to further categorize FAP expression in tumor immune cell populations, presumably using multicolor immunofluorescent staining, since we were unable to detect FAP expression by NK cells using cell surface-based flow cytometry approaches. Additionally, more studies are needed to determine the function of FAP in these FAP+ tumor macrophages to determine if it enhances proteolytic migration similar to FAP's function in NK cells which we described here.

The findings that human NK cells express FAP (FIG. 1D) has several clinical implications for FAP-targeted therapies. For example, an anti-FAP/IL-2 fusion protein is currently in clinical trials (NCT02627274). The proposed mechanism of action of this drug is that it targets IL-2 to FAP expressing tumor stroma, thereby limiting on-target, off-site toxicities associated with IL-2 cytokine therapy. Our findings that FAP is expressed on the NK cell surface suggests that and anti-FAP/IL-2 fusion protein may also target IL-2 directly to NK cells, enhancing NK cell activation and potentially tumor clearance. Since we were unable to detect FAP on the NK cell surface by flow cytometry, it is plausible that the anti-FAP construct in the anti-FAP-IL-2 fusion protein targets a similar epitope as the anti-FAP antibody we used, and therefore would not target IL-2 to NK cells. Our inability to detect FAP on NK cells by flow may be a byproduct of masked epitope or altered FAP structure, which would render nearly all anti-FAP antibodies unusable. Alternatively, this may be an antibody-specific problem and other anti-FAP antibodies would bind FAP on NK cells. Future studies are needed to determine if the anti-FAP/IL-2 fusion protein currently in clinical trials can bind to FAP on the NK cell surface.

Anti-FAP CAR therapies are also in development to treat conditions such as cardiac fibrosis (22), malignant pleural mesothelioma (62), lung adenocarcinoma (63) and other cancers (64). Our data suggest that anti-FAP CAR cells may also be useful in NK cell malignancies such as aggressive NK-cell leukemia if the anti-FAP portion was able to bind FAP on NK cells. There are potential caveats to the clinical use of anti-FAP CAR T cells. In one study, anti-FAP CAR T cells failed to regulate murine tumor growth and induced lethal bone toxicity and cachexia, potentially through the lysis of multipotent bone marrow stromal cells (65). It is plausible that an anti-FAP CAR T cell could induce NK cell lysis, resulting in NK cell leukopenia in humans only, therefore this toxicity would be missed in preclinical murine models. For cancer immunotherapy, an ideal anti-FAP CAR would be engineered to target FAP expression by fibroblasts and spare NK cells. Our findings that the anti-FAP antibody we used had variable binding to fibroblasts (i.e. PSCs) versus NK cells suggest this type of anti-FAP CAR engineering is feasible. It should be noted that Gulati et al. performed the first-in-human trial of an anti-FAP CAR T cell therapy, and demonstrated that a FAP CAR T cell therapy induced stable disease for 1 year in a patient with malignant pleural mesothelioma without any treatment-terminating toxicities (62).

Our findings that FAP regulates NK cell tissue infiltration (FIGS. 5 and 6) has clinical implications. FAP inhibitors, such as Cpd60, could reduce NK cell infiltration into tissues in diseases characterized by excessive NK cell content. These results also imply the potential value of engineering NK cells that overexpress FAP, enhancing NK cell tumor infiltration, which in turn would enhance tumor cell lysis.

Other strategies aimed at enhancing NK cell infiltration into tumors rely on manipulating chemokine/receptor pathways. One approach is to process NK cells in such a way that they have enhanced migratory phenotypes. For example, Wennerberg et al demonstrated that ex vivo expanded NK cells express higher levels of chemokine receptor CXCR3 than unexpanded NK cells. The expanded NK cells in turn had increased migration towards CXCL10 expressing melanomas (18). They suggested that autologous NK cell therapy could be improved by expanding the NK cells prior to reinjection to enhance tumor homing. Another approach is to engineer NK cells to enhance their migration. Kremer et al engineered NK cells to overexpress CXCR2, a chemokine receptor. They showed that CXCR2 overexpressing NK cells had enhanced trafficking towards and lysis of renal cell carcinoma cells in vitro (19). These findings suggest that strategies to enhance NK cell migration are both feasible and may have clinical applicability. However, chemokine pathway-altering strategies have built-in limitations. They require not only elevated expression of the chemokine receptor on NK cells, but also secretion and maintenance of chemoattractants by the tumor. Additionally, many chemoattractants recruit multiple immune cell types, including immunosuppressive cells. For example, CXCL10 is a chemoattractant for cytotoxic T lymphocytes and NK cells, but also for regulatory T cells (66). We postulate that the ideal migration-altering therapeutic approach would increase cytotoxic immune cell infiltration in tumor masses, without influencing or even reducing immunosuppressive immune cell content in the TME. Since inhibiting FAP reduces NK cell tumor infiltration and lysis (FIG. 6), we therefore speculate that the inverse is true and that engineering NK cells to overexpress FAP, either in autologous NK cell or CAR-NK therapies, could increase NK cell tumor infiltration and lysis. This approach is independent of tumor-associated factors, such as chemoattractant secretion, and would not be expected to induce the infiltration or expansion of immunosuppressive cell populations into the tumor microenvironment. Since proteolytic migration is required for NK cell killing of malignant cells (FIG. 6), the ability to alter protease expression or activity to enhance NK cell tumor infiltration represents a potentially promising approach to altering NK cell anti-tumor activity. Future studies are needed to explore the benefit of FAP-overexpressing NK cells in preclinical models and in clinical studies, and to determine what, if any, toxicities they induce.

This work also demonstrates the feasibility of studying human NK cell migration using physiologically relevant approaches in model systems. Human immune cell intravasation/extravasation studies often rely on artificial endothelial cell membranes. By using a zebrafish model, we were able to visualize and quantify human NK cell extravasation in vivo (FIG. 4). Van den Berg et al. demonstrated zebrafish models could be combined with advanced microscopy techniques, such as correlative light and electron microscopy, to visualize zebrafish immune cell extravasation (46). We have built upon this work and demonstrated that human NK cells can be inoculated into zebrafish embryos without apparent toxicities to the cells or the fish. We believe this model fills a current gap in available methodologies to investigate human immune cell migratory phenotypes in vivo. We also demonstrated that heterotypic spheroids, comprised of stromal producing cells and cancer cell lines, can be used to assess the impact of tumor matrix on immune cell migration (FIG. 5) complementing the less physiologic yet more controllable approach of embedding cells in 3D matrices.

There are numerous methods by which the immune cells of the present invention may be genetically modified to overexpress fibroblast activation (FAP) protein. One exemplary method is genetic transformation, a process by which the genetic material carried by an individual cell is altered by the incorporation of foreign (exogenous) DNA into its genome. Standard techniques may be used for recombinant DNA, oligonucleotide synthesis, and tissue culture and transformation (e.g., electroporation and lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual, 2d ed. (Cold Spring Harbor Laboratory Press, Cold Spring Harbor, N.Y. 1989).

The disclosed composition(s) may be incorporated into a pharmaceutical composition suitable for administration to a subject (such as a patient, which may be a human or non-human). The pharmaceutical compositions may comprise a carrier (e.g., a pharmaceutically acceptable carrier). Any suitable carrier can be used within the context of the disclosure, and such carriers are well known in the art. The choice of carrier will be determined, in part, by the particular use of the composition (e.g., administration to an animal) and the particular method used to administer the composition. In some embodiments, the administering is performed by adoptive cell transfer. In other embodiments, the genetically modified immune cells are administered by direct delivery to a tumor bed by injection. Accordingly, there is a wide variety of suitable formulations of the composition of the present invention.

In some embodiments, the invention provides a pharmaceutical composition comprising a genetically modified immune cell of the invention, or a population of genetically modified cells of the invention, and a pharmaceutical carrier. Such pharmaceutical compositions can be prepared in accordance with known techniques. See, e.g., Remington, The Science And Practice of Pharmacy (21^{st} ed. 2005). In the manufacture of a pharmaceutical formulation according to the invention, cells are typically mixed with a pharmaceutically acceptable carrier and the resulting composition is administered to a subject. The carrier must be acceptable in the sense of being compatible with any other ingredients in the formulation and must not be deleterious to the subject. In some embodiments, pharmaceutical compositions of the invention can further comprise one or more additional therapeutic agents useful in the treatment of a disease in the subject. In other embodiments, pharmaceutical compositions of the invention can further include biological molecules, such as cytokines or chemokines that promote anti-tumor activity, for example, through mediation of T-cell or NK-cell activity. Pharmaceutical compositions comprising genetically modified cells of the invention can be administered in the same composition as an additional agent or biological molecule or, alternatively, can be co-administered in separate compositions.

Additional therapeutic agent(s) may be administered simultaneously or sequentially with the disclosed genetically modified immune cells, inhibitors, and compositions. Sequential administration includes administration before or after the disclosed genetically modified immune cells and inhibitors. In some embodiments, the additional therapeutic agent or agents may be administered in the same composition as the disclosed genetically modified immune cells or inhibitors. In other embodiments, there may be an interval of time between administration of the additional therapeutic agent and the disclosed genetically modified immune cells or inhibitors. In some embodiments, administration of an additional therapeutic agent with a disclosed genetically modified immune cells or inhibitors may allow lower doses of the other therapeutic agents and/or administration at less frequent intervals. When used in combination with one or more other active ingredients, the genetically modified immune cells or inhibitors of the disclosure and the other active ingredients may be used in lower doses than when each is used singly. Accordingly, the pharmaceutical compositions of the disclosure include those that contain one or more other active ingredients, in addition to genetically modified immune cells or inhibitors of the disclosure. The above combinations include combinations of genetically modified immune cells or inhibitors of the disclosure not only with one other active compound, but also with two or more other active compounds. For example, the compound of the disclosure may be combined with a variety of drugs to treat cancer. As used herein the specification, “a” or “an” may mean one or more. As used herein in the claim(s), when used in conjunction with the word “comprising,” the words “a” or “an” may mean one or more than one. The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. Throughout this application, the term “about” and “approximately” are used to indicate that a value includes the inherent variation of error for the device, the method being employed to determine the value, or the variation that exists among the study subjects.

### REFERENCES

1. Vivier, E., E. Tomasello, M. Baratin, T. Walzer, and S. Ugolini. 2008. Functions of natural killer cells. Nat. Immunol. 9: 503-510. 2. Deniz, G., G. Erten, U. C. Kücüksezer, D. Kocacik, C. Karagiannidis, E. Aktas, C. A. Akdis, and M. Akdis. 2008. Regulatory NK Cells Suppress Antigen-Specific T Cell Responses. J. Immunol. 180: 850-857. 3. Walzer, T., M. Dalod, S. H. Robbins, L. Zitvogel, and E. Vivier. 2005. Natural-killer cells and dendritic cells: “L′union fait la force.” Blood 106: 2252-2258. 4. Shi, F. D., H. B. Wang, H. Li, S. Hong, M. Taniguchi, H. Link, L. Van Kaer, and H. G. Ljunggren. 2000. Natural killer cells determine the outcome of B cell-mediated autoimmunity. Nat. Immunol. 1: 245-251. 5. Shimoda, S., S. Hisamoto, K. Harada, S. Iwasaka, Y. Chong, M. Nakamura, Y. Bekki, T. Yoshizumi, K. Shirabe, T. Ikegami, Y. Maehara, X.-S. He, M. E. Gershwin, and K. Akashi. 2015. Natural killer cells regulate T cell immune responses in primary biliary cirrhosis. 595 Hepatology 62: 1817-1827. 6. Belyakova, K. L., O. I. Stepanova, A. R. Sheveleva, V. A. Mikhailova, D. I. Sokolov, and S. A. Sel′kov. 2019. Interaction of NK Cells, Trophoblast, and Endothelial Cells during Angiogenesis. Bull. Exp. Biol. Med. 167: 169-176. 7. Cursons, J., F. Souza-Fonseca-Guimaraes, M. Foroutan, A. Anderson, F. Hollande, S. Hediyeh-Zadeh, A. Behren, N. D. Huntington, and M. J. Davis. 2019. A gene signature predicting natural killer cell infiltration and improved survival in melanoma patients. Cancer Immunol. Res. 7: 1162-1174. 8. Li, B., Y. Jiang, G. Li, G. A. Fisher, and R. Li. 2020. Natural killer cell and stroma abundance are independently prognostic and predict gastric cancer chemotherapy benefit. JCI Insight 5. 9. Lee, H., C. Quek, I. Silva, A. Tasker, M. Batten, H. Rizos, S. Y. Lim, T. Nur Gide, P. Shang, G. H. Attrill, J. Madore, J. Edwards, M. S. Carlino, A. Guminski, R. P. M. Saw, J. F. Thompson, P. M. Ferguson, U. Palendira, A. M. Menzies, G. V. Long, R. A. Scolyer, and J. S. Wilmott. 2019. Integrated molecular and immunophenotypic analysis of NK cells in anti-PD-1 treated metastatic melanoma patients. Oncoimmunology 8: e1537581. 10. Davis-Marcisak, E., A. Fitzgerald, M. Kessler, L. Danilova, E. Jaffee, N. Zaidi, L. Weiner, 611 and E. Fertig. 2020. A novel mechanism of natural killer cell response to anti-CTLA-4 therapy identified by integrative analysis of mouse and human tumors. bioRxiv 2020.05.31.125625. 11. Barry, K., J. Hsu, M. Broz, F. Cueto, M. Binnewies, and A. Combes. 2018. A natural killer-dendritic cell axis defines checkpoint therapy-responsive tumor microenvironments. Nat. Med. 24: 1178-91. 12. Souza-Fonseca-Guimaraes, F., J. Cursons, and N. D. Huntington. 2019. The Emergence of Natural Killer Cells as a Major Target in Cancer Immunotherapy. Trends Immunol. 40. 13. Sakamoto, N., T. Ishikawa, S. Kokura, T. Okayama, K. Oka, M. Ideno, F. Sakai, A. Kato, M. Tanabe, T. Enoki, J. Mineno, Y. Naito, Y. Itoh, and T. Yoshikawa. 2015. Phase I clinical trial of autologous NK cell therapy using novel expansion method in patients with advanced digestive cancer. J. Transl. Med. 13: 277. 14. Liu, E., D. Marin, P. Banerjee, H. A. Macapinlac, P. Thompson, R. Basar, L. Nassif Kerbauy, B. Overman, P. Thall, M. Kaplan, V. Nandivada, I. Kaur, A. Nunez Cortes, K. Cao, M. Daher, C. Hosing, E. N. Cohen, P. Kebriaei, R. Mehta, S. Neelapu, Y. Nieto, M. Wang, W. Wierda, M. Keating, R. Champlin, E. J. Shpall, and K. Rezvani. 2020. Use of CAR-Transduced Natural Killer Cells in CD19-Positive Lymphoid Tumors. N. Engl. J. Med. 382: 545-553. 15. Fayette, J., G. Lefebvre, M. R. Posner, J. Bauman, S. Salas, C. Even, E. Saada-Bouzid, T. Seiwert, D. Colevas, F. Calmels, R. Zerbib, A. Boyer Chammard, and R. Cohen. 2018. Results of a phase II study evaluating monalizumab in combination with cetuximab in previously treated recurrent or metastatic squamous cell carcinoma of the head and neck (R/M SCCHN). Ann. Oncol. 29: viii374. 16. Sarhan, D., L. Brandt, M. Felices, K. Guldevall, T. Lenvik, P. Hinderlie, J. Curtsinger, E. Warlick, S. R. Spellman, B. R. Blazar, D. J. Weisdorf, S. Cooley, D. A. Vallera, B. Onfelt, and J. S. Miller. 2018. 161533 TriKE stimulates NK-cell function to overcome myeloid-derived suppressor cells in MDS. Blood Adv. 2: 1459-1469. 17. Felices, M., S. Chu, B. Kodal, L. Bendzick, C. Ryan, A. J. Lenvik, K. L. M. Boylan, H. C. Wong, A. P. N. Skubitz, J. S. Miller, and M. A. Geller. 2017. IL-15 super-agonist (ALT-803) enhances natural killer (NK) cell function against ovarian cancer. Gynecol. Oncol. 145: 453-461. 18. Wennerberg, E., V. Kremer, R. Childs, and A. Lundqvist. 2014. CXCL10-induced migration of adoptively transferred human natural killer cells toward solid tumors causes regression of tumor growth in vivo. Cancer Immunol. Immunother. 64: 225-235. 19. Kremer, V., M. A. Ligtenberg, R. Zendehdel, C. Seitz, A. Duivenvoorden, E. Wennerberg, E. Colon, A.-H. Scherman-Plogell, and A. Lundqvist. 2017. Genetic engineering of human NK cells to express CXCR2 improves migration to renal cell carcinoma. J. Immunother. Cancer 5:73. 20. Kameritsch, P., and J. Renkawitz. 2020. Principles of Leukocyte Migration Strategies. Trends Cell Biol. 30: 818-832. 21. Fitzgerald, A. A., and L. M. Weiner. 2020. The role of fibroblast activation protein in health and malignancy. Cancer Metastasis Rev. 39: 783-803. 22. Aghajanian, H., T. Kimura, J. G. Rurik, A. S. Hancock, M. S. Leibowitz, L. Li, J. Scholler, J. Monslow, A. Lo, W. Han, T. Wang, K. Bedi, M. P. Morley, R. A. Linares Saldana, N. A. Bolar, K. McDaid, C. A. Assenmacher, C. L. Smith, D. Wirth, C. H. June, K. B. Margulies, R. Jain, E. Pure, S. M. Albelda, and J. A. Epstein. 2019. Targeting cardiac fibrosis with engineered T cells. Nature 573: 430-433. 23. Busek, P., R. Mateu, M. Zubal, L. Kotackova, and A. Sedo. 2018. Targeting fibroblast activation protein in cancer—Prospects and caveats. Front. Biosci. (Landmark Ed. 23: 1933-1968. 24. Aldeghaither, D. S., D. J. Zahavi, J. C. Murray, E. J. Fertig, G. T. Graham, Y.-W. Zhang, A. O'Connell, J. Ma, S. A. Jablonski, and L. M. Weiner. 2019. A mechanism of resistance to antibody-targeted immune attack. Cancer Immunol. Res. 7. 25. Somanchi, S. S., V. V. Senyukov, C. J. Denman, and D. A. Lee. 2010. Expansion, purification, and functional assessment of human peripheral blood NK cells. J. Vis. Exp. 2540. 26. Iqbal, J., D. D. Weisenburger, A. Chowdhury, M. Y. Tsai, G. Srivastava, T. C. Greiner, C. Kucuk, K. Deffenbacher, J. Vose, L. Smith, W. Y. Au, S. Nakamura, M. Seto, J. Delabie, F. Berger, F. Loong, Y. H. Ko, I. Sng, X. Liu, T. P. Loughran, J. Armitage, and W. C. Chan. 2011. Natural killer cell lymphoma shares strikingly similar molecular features with a group of non-hepatosplenic γδ T-cell lymphoma and is highly sensitive to a novel aurora kinase A inhibitor in vitro. Leukemia 25: 348-358. 27. Gautier, L., L. Cope, B. M. Bolstad, and R. A. Irizarry. 2004. affy—analysis of Affymetrix 670 GeneChip data at the probe level. Bioinformatics 20: 307-315. 28. Gu, Z., R. Eils, and M. Schlesner. 2016. Complex heatmaps reveal patterns and correlations in multidimensional genomic data. Bioinformatics 32: 2847-2849. 29. Ritchie, M. E., B. Phipson, D. Wu, Y. Hu, C. W. Law, W. Shi, and G. K. Smyth. 2015. Limma powers differential expression analyses for RNA-sequencing and microarray studies. 675
Nucleic Acids Res. 43: e47. 30. Mi, H., A. Muruganujan, J. T. Casagrande, and P. D. Thomas. 2013. Large-scale gene function analysis with the panther classification system. Nat. Protoc. 8: 1551-1566. 31. Lee, B. J., and E. M. Mace. 2017. Acquisition of cell migration defines NK cell differentiation from hematopoietic stem cell precursors. Mol. Biol. Cell 28: 3573-3581. 32. Lee, B. J., E. Hegewisch Solloa, M. J. Shannon, and E. M. Mace. 2020. Generation of cell-1 derived matrices that support human NK cell migration and differentiation. J. Leukoc. Biol. 108: 1369-1378. 33. Schindelin, J., I. Arganda-Carreras, E. Frise, V. Kaynig, M. Longair, T. Pietzsch, S. Preibisch, C. Rueden, S. Saalfeld, B. Schmid, J. Y. Tinevez, D. J. White, V. Hartenstein, K. Eliceiri, P. Tomancak, and A. Cardona. 2012. Fiji: An open-source platform for biological-image analysis. Nat. Methods 9: 676-682. 34. Lin, Y. N., A. Nasir, S. Camacho, D. L. Berry, M. O. Schmidt, G. W. Pearson, A. T. Riegel, and A. Wellstein. 2020. Monitoring cancer cell invasion and t-cell cytotoxicity in 3d culture. J. Vis. Exp. 2020: 1-19. 35. Westcott, J. M., S. Camacho, A. Nasir, M. E. Huysman, R. Rahhal, T. T. Dang, A. T. Riegel, R. A. Brekken, and G. W. Pearson. 2020. ΔNp63-Regulated Epithelial-to-Mesenchymal Transition State Heterogeneity Confers a Leader-Follower Relationship That Drives Collective Invasion. Cancer Res. 80: 3933-3944. 36. Watt, J., and H. M. Kocher. 2013. The desmoplastic stroma of pancreatic cancer is a barrier to immune cell infiltration. Oncoimmunology 2. 37. Levy, M. T., G. W. McCaughan, C. A. Abbott, J. E. Park, A. M. Cunningham, E. Müller, W. 697 J. Rettig, and M. D. Gorrell. 1999. Fibroblast activation protein: A cell surface dipeptidyl peptidase and gelatinase expressed by stellate cells at the tissue remodelling interface inhuman cirrhosis. Hepatology 29: 1768-1778. 38. Barretina, J., G. Caponigro, N. Stransky, K. Venkatesan, A. A. Margolin, S. Kim, C. J. Wilson, J. Lehir, G. V. Kryukov, D. Sonkin, A. Reddy, M. Liu, L. Murray, M. F. Berger, J. E. Monahan, P. Morais, J. Meltzer, A. Korejwa, J. Jane-Valbuena, F. A. Mapa, J. Thibault, E. Bric-Furlong, P. Raman, A. Shipway, I. H. Engels, J. Cheng, G. K. Yu, J. Yu, P. Aspesi, M. de Silva, K. Jagtap, M. D. Jones, L. Wang, C. Hatton, E. Palescandolo, S. Gupta, S. Mahan, C. Sougnez, R. C. Onofrio, T. Liefeld, L. MacConaill, W. Winckler, M. Reich, N. Li, J. P. Mesirov, S. B. Gabriel, G. Getz, K. Ardlie, V. Chan, V. E. Myer, B. L. Weber, J. Porter, M. Warmuth, P. Finan, J. L. Harris, M. Meyerson, T. R. Golub, M. P. Morrissey, W. R. Sellers, R. Schlegel, and L. A. Garraway. 2012. The Cancer Cell Line Encyclopedia enables predictive modelling of anticancer 709 drug sensitivity. Nature 483: 603-607. 39. Goda, S., H. Inoue, H. Umehara, M. Miyaji, Y. Nagano, N. Harakawa, H. Imai, P. Lee, J. B. 711
Macarthy, T. Ikeo, N. Domae, Y. Shimizu, and J. Iida. 2006. Matrix metalloproteinase-1 produced by human CXCL12-stimulated natural killer cells. Am. J. Pathol. 169: 445-458. 40. Kitson, R. P., P. M. Appasamy, U. Nannmark, P. Albertsson, M. K. Gabauer, and R. H. Goldfarb. 1998. Matrix metalloproteinases produced by rat 1L-2-activated NK cells. J. Immunol. 160: 4248-4253. 41. Goldfarb Nannmark, R. H., P. H. Basse, P. J. K. Kuppen, M. E. Myoung, H. Kim, and R. P. 717 Kitson. 2000. Cells and Their Inhibitors Metalloproteinases of IL-2-Activated NK Secreted and 718 Membrane-Associated Matrix. J Immunol Ref 164: 5883-5889.
42. Jansen, K., L. Heirbaut, R. Verkerk, J. D. Cheng, J. Joossens, P. Cos, L. Maes, A.-M. Lambeir, I. De Meester, K. Augustyns, and P. Van Der Veken. 2014. Extended Structure-Activity Relationship and Pharmacokinetic Investigation of (4-Quinolinoyl)glycyl-2-cyanopyrrolidine Inhibitors of Fibroblast Activation Protein (FAP). J. Med. Chem. 57: 3053-723 3074. 43. Mace, E. M., J. T. Gunesch, A. Dixon, and J. S. Orange. 2016. Human NK cell development requires CD56-mediated motility and formation of the developmental synapse. Nat. Commun. 7. 44. Hwang, H. J., M. S. Oh, D. W. Lee, and H. J. Kuh. 2019. Multiplex quantitative analysis of stroma-mediated cancer cell invasion, matrix remodeling, and drug response in a 3D co-culture model of pancreatic tumor spheroids and stellate cells. J. Exp. Clin. Cancer Res. 38: 1-14. 45. Ware, M. J., V. Keshishian, J. J. Law, J. C. Ho, C. A. Favela, P. Rees, B. Smith, S. Mohammad, R. F. Hwang, K. Rajapakshe, C. Coarfa, S. Huang, D. P. Edwards, S. J. Corr, B. Godin, and S. A. Curley. 2016. Generation of an in vitro 3D PDAC stroma rich spheroid model. Biomaterials 108: 129-142. 46. van den Berg, M. C. W., L. MacCarthy-Morrogh, D. Carter, J. Morris, I. Ribeiro Bravo, Y. Feng, and P. Martin. 2019. Proteolytic and Opportunistic Breaching of the Basement Membrane Zone by Immune Cells during Tumor Initiation. Cell Rep. 27: 2837-2846.e4. 47. Kurz, A. R. M., M. Pruenster, I. Rohwedder, M. Ramadass, K. Schafer, U. Harrison, G. Gouveia, C. Nussbaum, R. Immler, J. R. Wiessner, A. Margraf, D. S. Lim, B. Walzog, S. Dietzel, M. Moser, C. Klein, D. Vestweber, R. Haas, S. D. Catz, and M. Sperandio. 2016. MST1-dependent vesicle trafficking regulates neutrophil transmigration through the vascular basement membrane. J. Clin. Invest. 126: 4125-4139. 48. Kim, M. H., R. P. Kitson, P. Albertsson, U. Nannmark, P. H. Basse, P. J. K. Kuppen, M. E. Hokland, and R. H. Goldfarb. 2000. Secreted and Membrane-Associated Matrix Metalloproteinases of IL-2-Activated NK Cells and Their Inhibitors. J. Immunol. 164: 5883-5889. 49. Zeng, L., S. An, and E. J. Goetzl. 1996. Selective regulation of RNK-16 cell matrix metalloproteinases by the EP4 subtype of prostaglandin E2 receptor. Biochemistry 35: 7159-7164. 50. Putz, E. M., A. J. Mayfosh, K. Kos, D. S. Barkauskas, K. Nakamura, L. Town, K. J. Goodall, D. Y. Yee, I. K. H. Poon, N. Baschuk, F. Souza-Fonseca-Guimaraes, M. D. Hulett, and M. J. Smyth. 2017. NK cell heparanase controls tumor invasion and immune surveillance. J. Clin. Invest. 127: 2777-2788. 51. Prakash, M. D., M. A. Munoz, R. Jain, P. L. Tong, A. Koskinen, M. Regner, O. Kleifeld, B. Ho, M. Olson, S. J. Turner, P. Mrass, W. Weninger, and P. I. Bird. 2014. Granzyme B promotes cytotoxic lymphocyte transmigration via basement membrane remodeling. Immunity 41: 960-972. 52. Iwasa, S., X. Jin, K. Okada, M. Mitsumata, and A. Ooi. 2003. Increased expression of seprase, a membrane-type serine protease, is associated with lymph node metastasis in human colorectal cancer. Cancer Lett. 199: 91-8. 53. Kelly, T., S. Kechelava, T. L. Rozypal, K. W. West, and S. Korourian. 1998. Seprase, a membrane-bound protease, is overexpressed by invasive ductal carcinoma cells of human breast cancers. Mod. Pathol. 11: 855-63. 54. Mori, Y., K. Kono, Y. Matsumoto, H. Fujii, T. Yamane, M. Mitsumata, and W.-T. Chen. 2004. The expression of a type II transmembrane serine protease (Seprase) in human gastric carcinoma. Oncology 67: 411-9. 55. Monsky, W. L., C. Y. Lin, A. Aoyama, T. Kelly, S. K. Akiyama, S. C. Mueller, and W. T. Chen. 1994. A potential marker protease of invasiveness, seprase, is localized on invadopodia of human malignant melanoma cells. Cancer Res. 54: 5702-10. 56. Arnold, J. N., L. Magiera, M. Kraman, and D. T. Fearon. 2014. Tumoral immune suppression by macrophages expressing fibroblast activation protein-α and heme oxygenase-1. Cancer Immunol. Res. 2: 121-6. 57. Tchou, J., P. J. Zhang, Y. Bi, C. Satija, R. Marjumdar, T. L. Stephen, A. Lo, H. Chen, C. 772
Mies, C. H. June, J. Conejo-Garcia, and E. Pure. 2013. Fibroblast activation protein expression by stromal cells and tumor-associated macrophages in human breast cancer. Hum. Pathol. 44: 2549-2557. 58. Ghersi, G., Q. Zhao, M. Salamone, Y. Yeh, S. Zucker, and W.-T. Chen. 2006. The protease complex consisting of dipeptidyl peptidase IV and seprase plays a role in the migration and invasion of human endothelial cells in collagenous matrices. Cancer Res. 66: 4652-61. 59. Wäster, P., I. Rosdahl, B. F. Gilmore, and O. Seifert. 2011. Ultraviolet exposure of melanoma cells induces fibroblast activation protein-α in fibroblasts: Implications for melanoma invasion, University of Crete, Faculty of Medicine, Laboratory of Clinical Virology. 60. Kennedy, A., H. Dong, D. Chen, and W.-T. Chen. 2009. Elevation of seprase expression and promotion of an invasive phenotype by collagenous matrices in ovarian tumor cells. Int. J. Cancer 124: 27-35. 61. Ruan, P., Z. Tao, and A. Tan. 2018. Low expression of miR-30a-5p induced the proliferation and invasion of oral cancer via promoting the expression of FAP. Biosci. Rep. 38: BSR20171027. 62. Schuberth, P. C., C. Hagedorn, S. M. Jensen, P. Gulati, M. van den Broek, A. Mischo, A. Soltermann, A. Jüngel, O. Marroquin Belaunzaran, R. Stahel, C. Renner, and U. Petrausch. 2013. Treatment of malignant pleural mesothelioma by fibroblast activation protein-specific re-directed T cells. J. Transl. Med. 11: 1-11. 63. Kakarla, S., K. K. H. Chow, M. Mata, D. R. Shaffer, X. T. Song, M. F. Wu, H. Liu, L. L. Wang, D. R. Rowley, K. Pfizenmaier, and S. Gottschalk. 2013. Antitumor effects of chimeric receptor engineered human T cells directed to tumor stroma. Mol. Ther. 21: 1611-1620. 64. Santos, A. M., J. Jung, N. Aziz, J. L. Kissil, and E. Pure. 2009. Targeting fibroblast activation protein inhibits tumor stromagenesis and growth in mice. J. Clin. Invest. 119: 3613-796 3625. 65. Tran, E., D. Chinnasamy, Z. Yu, R. A. Morgan, C. C. R. Lee, N. P. Restifo, and S. A. Rosenberg. 2013. Immune targeting of fibroblast activation protein triggers recognition of multipotent bone marrow stromal cells and cachexia. J. Exp. Med. 210: 1065-1068. 66. Lunardi, S., S. Y. Lim, R. J. Muschel, and T. B. Brunner. 2015. IP-10/CXCL10 attracts regulatory T cells: Implication for pancreatic cancer. Oncoimmunology 4: 1-3.

All publications, patents and patent applications cited herein are hereby incorporated by reference as if set forth in their entirety herein. The foregoing description and drawings should be considered as illustrative only of the principles of the disclosure. The disclosure is not intended to be limited by the preferred embodiment and may be implemented in a variety of ways that will be clear to one of ordinary skill in the art. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

1. A pharmaceutical composition comprising genetically modified immune cells, wherein the modified immune cells overexpress fibroblast activation protein (FAP).

2. The pharmaceutical composition of claim 1, wherein the genetically modified immune cells are natural killer (NK) cells, T-cells, or a combination thereof.

3. The pharmaceutical composition of claim 2, wherein the T-cells are CD4 T-cells, CD8 T-cells, or a combination thereof.

4. The pharmaceutical composition of claim 2, wherein the natural killer cells are selected from NK92, NK92-GFP, NKL, YT, KHYG-1, NK92-CD16V, or a combination thereof.

5. The pharmaceutical composition of claim 2, wherein the genetically modified natural killer cells are derived from normal human donors.

6. The pharmaceutical composition of claim 2, wherein, prior to genetic modification, the natural killer cells are isolated from peripheral blood, pluripotent stem cells, or a combination thereof.

7. The pharmaceutical composition of claim 2, wherein the genetically modified natural killer cells are further modified to express a chimeric antigen receptor (CAR).

8. The pharmaceutical composition of claim 3, wherein the natural killer cells are further genetically modified to overexpress one or more chemokines.

9. The pharmaceutical composition of claim 4, wherein the one or more chemokines are CCL2, CCL5, CCL20, CXCL1, CXCL2, CXCL5, CXCL9, CXCL10, CXCL12, CXCL14, CXCL16, CXCL28, or a combination thereof.

10. The pharmaceutical composition of claim 5, wherein the one or more chemokines are overexpressed upon engagement with a cancer cell.

11. The pharmaceutical composition of any of claims 1 to 5, wherein the genetic modification of the immune cells is performed by transformation, transfection, or transduction.

12. The pharmaceutical composition of any of claims 1 to 6, further comprising a pharmaceutically acceptable carrier.

13. The pharmaceutical composition of any of claims 1 to 12, wherein the composition is therapeutically effective against a cancer.

14. The pharmaceutical composition of claim 13, wherein the cancer is pancreatic cancer.

15. The pharmaceutical composition of claim 14, wherein the pancreatic cancer is a pancreatic ductal adenocarcinoma (PDAC).

16. A method of disease treatment comprising: administering a pharmaceutical composition comprised of a therapeutically effective amount of genetically modified immune cells, wherein the genetically modified immune cells are altered to overexpress fibroblast activation protein (FAP).

17. The method of claim 16, wherein the disease is cancer.

18. The method of claim 17, wherein the cancer is pancreatic cancer.

19. The method of claim 18, wherein the pancreatic cancer is a pancreatic ductal adenocarcinoma (PDAC).

20. The method of any one of claims 16 to 19, wherein the genetically modified immune cells are natural killer (NK) cells, T-cells, or a combination thereof.

21. The method of claim 20, wherein the T-cells are CD4 T-cells, CD8 T-cells, or a combination thereof.

22. The method of claim 20, wherein the genetically modified natural killer cells are derived from normal human donors.

23. The pharmaceutical composition of claim 20, wherein, prior to genetic modification, the natural killer cells are isolated from peripheral blood, pluripotent stem cells, or a combination thereof.

24. The pharmaceutical composition of claim 20, wherein the genetically modified natural killer cells are further modified to express a chimeric antigen receptor (CAR).

25. The method of claim 20, wherein the natural killer cells are genetically modified to overexpress one or more chemokines.

26. The method of claim 21, wherein the one or more chemokines are CCL2, CCL5, CCL20, CXCL1, CXCL2, CXCL5, CXCL9, CXCL10, CXCL12, CXCL14, CXCL16, CXCL28, or a combination thereof.

27. The method of claim 25, wherein the one or more chemokines are overexpressed upon engagement with a cancer cell.

28. The method of any of claims 16 to 27, wherein the genetic modification of the immune cells is performed by transformation, transfection, or transduction.

29. The method of any of claims 16 to 28, wherein the pharmaceutical composition further comprises a pharmaceutically acceptable carrier.

30. The method of any of claims 15 to 29, wherein the pharmaceutical composition is administered directly into a cancerous tumor.

31. The method of any of claims 16 to 30, wherein the pharmaceutical composition is administered intravenously.

32. A method of enhancing the ability of immune cells to migrate into a tumor comprising genetically modifying the immune cells to overexpress fibroblast activation protein (FAP).

33. The method of claim 32, wherein the genetically modified immune cells are natural killer (NK) cells, T-cells, or a combination thereof.

34. The method of claim 33, wherein the genetically modified natural killer cells are derived from normal human donors.

35. The method of claim 33, wherein, prior to genetic modification, the natural killer cells are isolated from peripheral blood, pluripotent stem cells, or a combination thereof.

36. The method of claim 33, wherein the genetically modified natural killer cells are further modified to express a chimeric antigen receptor (CAR).

37. The method of claim 33, wherein the natural killer cells are genetically modified to overexpress one or more chemokines.

38. The method of claim 34, wherein the one or more chemokines are CCL2, CCL5, CCL20, CXCL1, CXCL2, CXCL5, CXCL9, CXCL10, CXCL12, CXCL14, CXCL16, CXCL28, or a combination thereof.

39. The method of claim 31, wherein the one or more chemokines are overexpressed upon engagement with a cancer cell.

40. The method of any of claims 32 to 39, wherein the genetic modifications of the immune cells are performed by transformation, transfection, or transduction.

41. The method of any of claims 32 to 40, further comprising administering the genetically modified immune cells to a patient suffering from cancer.

42. The method of claim 41, wherein the cancer is pancreatic cancer.

43. The method of claim 42, wherein the pancreatic cancer is a pancreatic ductal adenocarcinoma (PDAC).

44. A method of preparing one or more genetically modified immune cells comprising: transfecting a vector containing a gene for fibroblast activation protein (FAP) into one or more immune cells in a media; replicating the one or more immune cells transfected by the vector; and isolating the one more immune cells transfected by the vector that overexpresses fibroblast activation protein (FAP).

45. The method of claim 44, wherein the vector is viral.

46. The method of claim 44 or 45, wherein the one or more immune cells are natural killer (NK) cells, T-cells, or a combination thereof.

47. The method of claim 46, wherein the natural killer cells are genetically modified to overexpress one or more chemokines.

48. The method of claim 46, wherein the one or more chemokines are CCL2, CCL5, CCL20, CXCL1, CXCL2, CXCL5, CXCL9, CXCL10, CXCL12, CXCL14, CXCL16, CXCL28, or a combination thereof.

49. The method of claim 46, wherein the one or more chemokines are overexpressed upon engagement with a cancer cell.
